(19) United States
(12) Patent Application Publication
(10) Pub. No.: US 2021/0315882 A1
(43) Pub. Date: Oct. 14, 2021

(54) METHODS OF TREATING A PATIENT HAVING PARKINSON'S DISEASE

(71) Applicant: Sunovion Pharmaceuticals Inc., Marlborough, MA (US)

(72) Inventors: Thierry BILBAULT, Cambridge, MA (US); Bradford Armando NAVIA, Cambridge, MA (US); Charles Warren OLANOW, Cambridge, MA (US)

(21) Appl. No.: 17/344,314

(22) Filed: Jun. 10, 2021

Related U.S. Application Data
(63) Continuation of application No. PCT/US2020/015434, filed on Jan. 28, 2020.
(60) Provisional application No. 62/861,077, filed on Jun. 13, 2019.
(60) Provisional application No. 62/861,067, filed on Jun. 13, 2019.
(60) Provisional application No. 62/797,660, filed on Jan. 28, 2019.

(51) Int. Cl. A61K 31/473; A61K 9/00; A61P 25/16

(52) U.S. Cl. CPC A61K 31/473; A61P 25/16; A61K 9/006

(57) ABSTRACT

Methods of treating a patient having Parkinson's disease, including treating “OFF” episodes and improving motor function, by administering apomorphine or a pharmaceutically acceptable salt thereof are disclosed herein. Supratherapeutic doses of apomorphine may be used. Apomorphine therapy may be used in place of levodopa therapy, or apomorphine may be used to supplant or augment the first levodopa dose of the day. Apomorphine therapy may involve administration of two consecutive apomorphine doses at least 2 hours apart.

### CROSS-REFERENCE TO RELATED APPLICATIONS

This application claims priority to U.S. Provisional Patent Application No. 62/797,660, filed on Jan. 28, 2019, entitled Methods of Treating a Patient Having Parkinson's Disease; U.S. Provisional Patent Application No. 62/861,067, filed on Jun. 13, 2019, entitled Methods of Treating a Patient Having Parkinson's Disease; and U.S. Provisional Patent Application No. 62/861,077, filed on Jun. 13, 2019, entitled Methods of Improving Motor Function in a Patient Having an “OFF” Episode Associated with Parkinson's Disease, the contents of each of which are herein incorporated by reference in their entirety.

### FIELD

The present disclosure relates to methods of treating a patient having Parkinson's disease, e.g., treating “OFF” episodes associated with Parkinson's disease and improving motor function, by administering apomorphine or a pharmaceutically acceptable salt thereof.

### BACKGROUND

Parkinson's disease (PD) affects more than 1.5 million individuals in the United States. The symptoms of PD vary from patient to patient. The common primary symptoms are a paucity of movement and rigidity, characterized by an increased stiffness of voluntary skeletal muscles. Additional symptoms include resting tremor, slowness of movement (bradykinesia), poor balance, and walking problems. Common secondary symptoms include depression, sleep disturbance, dizziness, stooped posture, dementia, problems with speech, breathing, and swallowing. These symptoms become progressively worse with time, ultimately resulting in death.

There is a need for new apomorphine treatment regimens which are safe, effective, and easy to use for a patient having PD.

### SUMMARY

Disclosed herein are methods of treating a patient having Parkinson's disease, e.g., treating “OFF” episodes associated with Parkinson's disease and improving motor function, by administering apomorphine or a pharmaceutically acceptable salt thereof. In some embodiments the apomorphine is administered via the oral mucosa of a patient. In some embodiments the apomorphine is administered buccually. In some embodiments the apomorphine is administered sublingually.

In some embodiments, a patient population of apomorphine responders is selected for treatment by titrating patients with increasing dosages of apomorphine and selecting those that display a “Full On” response from an “Off” state at sublingual apomorphine dosages providing a Cmax of less than about 10 ng/mL.

In some embodiments, the present disclosure provides a method of treating a patient having Parkinson's disease comprising administering to the patient a supratherapeutic dose (as defined herein) of apomorphine or a pharmaceutically acceptable salt thereof. In some embodiments, the method improves motor function and reduces the incidence and severity of “OFF” episodes.

In some embodiments, the patient is administered apomorphine therapy in the absence of levodopa. In some embodiments, the present disclosure provides a method of treating Parkinson's disease in a patient receiving a levodopa regimen, the method comprising administering apomorphine or a pharmaceutically acceptable salt thereof, wherein the administering step supplants the first levodopa dose of the day. In some embodiments, second and further doses of levodopa are administered to the patient throughout the day after administration of apomorphine or a salt thereof. In some embodiments, the daily levodopa regimen does not begin for at least 90 minutes after administration of apomorphine or a salt thereof. In some embodiments, a supratherapeutic dose of apomorphine or salt thereof is administered to the patient. In some embodiments administration of apomorphine therapy replaces or is begun in lieu of levodopa therapy.

In some embodiments the patient is administered apomorphine therapy in combination with levodopa, e.g., augmenting levodopa therapy. In some embodiments, the present disclosure provides a method of treating Parkinson's disease in a patient receiving a levodopa regimen, the method comprising administering apomorphine or a pharmaceutically acceptable salt thereof, wherein the administering step augments the first levodopa dose of the day. In some embodiments, second and further doses of levodopa are administered to the patient throughout the day after administration of apomorphine or a salt thereof. In some embodiments, the apomorphine or salt thereof is administered concomitantly with the first levodopa dose of the day.

Other embodiments are described in paragraphs E1 to E70, A1 to A71, B1 to B23, and C1 to C15 herein.

### BRIEF DESCRIPTION OF THE DRAWINGS

FIG. 1 shows the change from baseline in MDS-UPDRS Part III Score up to 90 minutes post-dose at week 12 for the patients in the study of Example 1.

FIG. 2 shows the change from baseline in MDS-UPDRS Part III Score 30 minutes post-dose across weeks 0-12 for the patients in the study of Example 1.

FIG. 3 shows the mean change from baseline in QTcF (AQTcF) by Time Point for treatment, placebo, and moxifloxacin control groups in Example 2.

FIG. 4 is a forest plot of the time-matched and placebo-adjusted mean changes from baseline in QTcF (ΔΔQTcF) for the treatment group of Example 2, abbreviations in the figure: Baseline=Treatment Period 1 Dosing Visit 1 (P1V1); CL=confidence limit; MMRM=mixed model for repeated measures; QTcF=QT time corrected with Fridericia's method; SE=Standard Error.

FIG. 5 is a forest plot of the time-matched and placebo-adjusted mean changes from baseline in QTcF (ΔΔQTcF) for the moxifloxacin control group of Example 2, abbreviations in the figure: Baseline=Treatment Period 1 Dosing Visit 1 (P1V1); CL=confidence limit; MMRM=mixed model for repeated measures; QTcF=QT time corrected with Fridericia's method; SE=Standard Error.

FIGS. 6A (15 minutes to 1 hr.), 6B (from 1 hr. to 8 hrs.) and 6C (from 8 hrs. to 24 hrs.) show the outlier analysis for QTcF (percentage and number of outliers by post-dose time-point and treatment/control group) in Example 2, wherein the number of participants in the ECG population=40, abbreviations in figure: QTcF=QT time corrected with Fridericia's method; y-axis represents percentage, bar-labels show number of subjects, Note: For QTcF>500 ms the baseline was less than or equal to 500 ms, for QTcF >480 ms the baseline was less than or equal to 480 ms, and for QTcF>450 ms, the baseline was less than or equal to 450 ms.

FIGS. 7A (15 minutes to 1 hr.), 7B (from 1 hr. to 8 hrs.) and 7C (from 8 hrs. to 24 hrs.) show the outlier analysis for QTcB (percentage and number of outliers by post-dose time-point and treatment/control group) in Example 2, wherein the number of participants in the ECG population=40, abbreviations in figure: QTcB=QT time corrected with Bazett's method; y-axis represents percentage, bar-labels show number of subjects, Note: For QTcF>500 ms the baseline was less than or equal to 500 ms, for QTcF >480 ms the baseline was less than or equal to 480 ms, and for QTcF>450 ms, the baseline was less than or equal to 450 ms.

FIGS. 8A (15 minutes to 45 mins.), 8B (from 45 mins. to 3 hrs.), 8C (from 3 hrs. to 12 hrs.) and 8D (from 12 hrs. to 24 hrs.) show the outlier analysis for HR, PR interval, QRS interval and QT interval (percentage and number of outliers by post-dose time-point and treatment/control group) in Example 2, wherein the number of participants in the ECG population=40, abbreviations in Figure: HR=heart rate; PR=time between start of P wave and start of QRS complex; QRS=time of QRS complex (Q, R, and S waves); QT=time between start of Q wave and end of T wave; y-axis represents percentage, bar-labels show number of subjects, Note: Bradycardiac event; post-dose HR<50 bpm and greater than or equal to 25% decrease from baseline. Tachycardic event: post-dose HR >100 BPM and greater than or equal to 25% increase from baseline. For QT >500 ms the baseline was less than or equal to 500 ms.

FIG. 9 shows a schematic diagram of a study design for treatment of Parkinson's disease patients in the “OFF” state with apomorphine sublingual film, abbreviations and terminology used in figure: Patient “OFF” vs “ON” training occurred as part of the levodopa challenge; any patient who could not differentiate between these states was deemed a screen failure; Notes on antinausea medication: Antinausea medication could be discontinued during the double-blind treatment phase at the investigator's discretion, abbreviations: BID=2 times per day; OUS=outside of the United States; PD=Parkinson's Disease; TID=3 times per day; TV=titration visit; US=United States; Note on 35 mg dose: Dose of 35 mg was given as 2 sublingual films consisting of 20 mg followed by 15 mg; abbreviation: APL=apomorphine sublingual film.

FIG. 10 shows a plot demonstrating the change in the Movement Disorder Society Unified Parkinson's Disease Rating Scale (MDS-UPDRS) Part III Motor Examination scores at multiple time points following administration of apomorphine sublingual film or levodopa to Parkinson's disease patients. Change is measured with respect to predose scores, abbreviations in figure: MDS-UPDRS=Movement Disorder Society Unified Parkinson's Disease Rating Scale; SE=Standard Error.

FIG. 11 shows a bar plot demonstrating the percentage of Parkinson's disease patients characterized as responders at multiple time points following administration of apomorphine sublingual film or levodopa.

FIG. 12 is a scheme showing the study design for an open-label titration phase for Parkinson's disease patients in the “OFF” state treated with an apomorphine sublingual film, Notes to Figure: During the open label titration phase, Use of antiemetics was prohibited and Titration visits were scheduled no more than 2 days apart; during titration visit nos. 3 to 6 patients, could receive the next-highest dose of apomorphine sublingual film within 4 hours if another “OFF” episode occurred that day.

FIG. 13 is a chart showing the distribution of apomorphine sublingual film doses (e.g., therapeutic and supratherapeutic doses) administered to Parkinson's disease patients. Note: abbreviation used in figure, “TV” means a titration visit.

FIG. 14 is a chart showing the change in the Movement Disorder Society Unified Parkinson's Disease Rating Scale (MDS-UPDRS) Part III scores following administration of therapeutic and supratherapeutic doses of apomorphine sublingual film in Parkinson's disease patients. Change is shown relative to the predose scores, abbreviations used in Figure: LS=least wquares; MDS-UPDRS=Movement Disorder Society Unified Parkinson's Disease Rating Scale; SE=Standard error.

FIG. 15A is a chart showing the percentage of patients achieving a FULL “ON” state over time in Parkinson's disease patients treated with therapeutic and supratherapeutic doses of an apomorphine sublingual film.

FIG. 15B is a chart showing the duration of the FULL “ON” state in Parkinson's disease patients treated with therapeutic and supratherapeutic doses of an apomorphine sublingual film.

FIG. 16 is a plot of MDS-UPDRS Part III score versus apomorphine blood plasma concentration showing the significant degree of interpatient variability.

FIG. 17 is a chart of the predicted change from baseline in MDS-UPDRS Part III score versus time at different doses of apomorphine sublingual film (APL-130277) based on the final updated Exposure-Response Efficacy Model discussed in Example 5.

### DETAILED DESCRIPTION

As used herein, “administering” or “administration” of apomorphine or a pharmaceutically acceptable salt thereof encompasses the delivery of apomorphine or a pharmaceutically acceptable salt thereof, or a prodrug or other pharmaceutically acceptable derivative thereof, to a patient using any suitable formulation or route of administration described herein. In some embodiments, apomorphine is administered to the oral mucosa of a patient (e.g., sublingual administration or buccal administration). In some embodiments, apomorphine is administered sublingually.

“Adverse events associated with subcutaneously administered apomorphine” are understood by a person of ordinary skill in the art as adverse events that are paradigmatic of subcutaneously administered apomorphine and include falling asleep during activities of daily living, somnolence, syncope, hypotension, orthostatic hypotension, hallucinations, psychotic behavior, dyskinesia, impulse control problems, coronary events, and QT prolongation.

The term “at risk,” when referring to an individual, refers to an individual who is at risk of developing a disorder to be treated or experiencing side effects associated with such treatment. This may be shown, for example, by one or more risk factors, which are measurable parameters that correlate with development of a disorder and are known in the art, or for example, a clinically significant rate of occurrence of an adverse event appearing among a population of patients receiving therapeutic treatment.

As used herein, a “clinically significant” risk of an adverse event refers to a risk that is greater than placebo by a statistically significant margin. When the risk from treatment of adverse events or a particular adverse event is less than, the same as, or about the same as placebo, the risk is not clinically significant. When the incidence of an adverse event is less than or equal to 2%, then the risk is not clinically significant, regardless of the incidence for a placebo population.

The term “Cmax,” as used herein, refers to an average observed maximum plasma concentration produced in a group of patients (e.g., 10 or more) receiving an apomorphine film of a particular dosage strength, thus providing a point of measurement in a patient population that accounts for individual patient variability in serum response to receipt of a particular dose of medicament, for example, sublingual administration of a formulation of the invention. In some embodiments, therapeutic dosages are described as the range of serum concentrations between Cₘₐₓ just sufficient to produce an “On” state in a patient population (determined, for example, by up-titration of a patient population experiencing an “Off” state) up to the Cₘₐₓ associated with producing adverse events, as described herein, in that patient population.

The term “delaying” development of a disorder, as used herein, means to defer, hinder, slow, stabilize, and/or postpone development of the disorder. Delay can be of varying lengths of time, depending on the history of the disease and/or the individual being treated.

The term “fewer adverse events,” as used herein, refers to an average observed number and severity of adverse events produced in a group of patients (e.g., 10 or more) receiving an apomorphine film in an amount sufficient to produce an “on” state following administration of apomorphine or a pharmaceutically acceptable salt thereof in comparison to the average number and severity of adverse events produced in a group of patients (e.g., 10 or more) receiving a unit dosage form producing a higher Cmax. In some embodiments, for some individual patients, the Cₘₐₓ can be greater than 10 mg/mL. In general, averaged over a patient population, the Cmax produced by the film can be from about 2.64 ng/mL to about 10 ng/mL (e.g., from 2.64 ng/mL to 9 ng/mL, 8 ng/mL, 7 ng/mL, 6 ng/mL, 5 ng/mL, or from 2.64 ng/mL to 4.7 ng/mL), optionally, with a Tmax of from 25 minutes to 70 minutes (e.g., from 30 minutes to 70 minutes, from 35 minutes to 60 minutes, from 30 minutes to 50 minutes, from 30 minutes to 40 minutes, or from 30 minutes to 70 minutes). Some pharmaceutical unit dosage forms thus can produce an “on” state and fewer adverse in a patient by providing Cmax of from 2.64 ng/mL to 7.1 ng/mL and Tmax of from 30 minutes to 50 minutes after administration of the pharmaceutical unit dosage form to the patient. Certain pharmaceutical unit dosage forms thus can produce an “on” state and fewer adverse in a patient by providing Cmax of from 2.64 ng/mL to 5.0 ng/mL and Tmax of from 25 minutes to 60 minutes after administration of the pharmaceutical unit dosage form to the patient. Pharmaceutical unit dosage forms thus can produce an “on” state and fewer adverse in a patient by providing Cmax of from 2.64 ng/mL to 4.7 ng/mL and Tmax of from 30 minutes to 60 minutes after administration of the pharmaceutical unit dosage form to the patient. Certain other pharmaceutical unit dosage forms thus can produce an “on” state and fewer adverse in a patient by providing Cmax of from 2.64 ng/mL to 5.0 ng/mL (e.g., from 2.64 ng/mL to 4.7 ng/mL) and Tmax of from 25 minutes to 40 minutes after administration of the pharmaceutical unit dosage form to the patient. The adverse events may be, e.g., somnolescence, nausea, yawning, headache, or hyperhidrosis.

As used herein “minimizing” adverse events refers to a statistically significant reduction in the incidence of adverse events in a patient population compared to the paradigmatic incidence of adverse events in a patient population treated with subcutaneously administered apomorphine. The corresponding risk of adverse events in a single patient is reduced accordingly.

The term “pH neutralizing agent,” as used herein, refers to any basic component present in the unit dosage forms. The pH neutralizing agents which can be used in the unit dosage forms include organic bases (e.g., pyridoxine, meglumine, lysine, Eudragit E, diethanolamine, glycine, citrate, acetate, histidine, N-methyl glucamine, or tris(hydroxymethyl)aminomethane), inorganic bases (e.g., oxides, hydroxides, carbonates, or phosphates), and mixtures thereof. The pH neutralizing agent is typically present in an amount sufficient to produce a solution having a pH of between 2.5 and 8.0, preferably between 4.5 and 6.5, when the unit dosage form is placed in 1 mL of unbuffered water at pH 7.

The terms “pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt,” as used herein refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of apomorphine include those derived from suitable inorganic and organic acids (e.g., acid addition salts) and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions are quite acceptable as synthetic intermediates. Thus X may be pharmaceutically undesirable anions, such as iodide, oxalate, trifluoromethanesulfonate and the like, when such salts are chemical intermediates.

The term “pharmaceutically acceptable excipient,” as used herein, includes, without limitation, any binder, filler, adjuvant, carrier, excipient, glidant, sweetening agent, diluent, buffer, permeation enhancer, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by the United States Food and Drug Administration as being acceptable for use in humans or domestic animals.

The term “prevention” or “preventing,” as used herein, refers to a regimen that protects against the onset of the disorder such that the clinical symptoms of the disorder do not develop. Accordingly, “prevention” relates to administration of a therapy to a patient before signs of the diseases are detectable in the patient (for example, administration of a therapy in the absence of a detectable syndrome of the disorder). The patient may be an individual at risk of developing the disorder.

The term “patient,” as used herein, refers to humans (i.e., a male or female). The “patient” may have independently been diagnosed with Parkinson's disease as defined herein, may currently be experiencing symptoms associated with Parkinson's disease or may have experienced symptoms in the past, may be at risk of developing Parkinson's disease, or may be reporting one or more of the symptoms of Parkinson's disease, even though a diagnosis may not have been made. The patient may be diagnosed as having Parkinson's disease through the use of techniques known in the art, e.g., a unified Parkinson's disease rating scale (UPDRS, e.g., Movement Disorder Society-Sponsored Revision of UPDRS (MDS-UPDRS)) or Hoehn or Yahr scale may be used. A person of ordinary skill in the art will appreciate that the term “patient” as used herein may refer to an individual, a population, or both. In some embodiments, the patient is a population of patients. In such embodiments, an individual patient may hypothetically exhibit certain adverse events that are, on average, not exhibited by a broader population, and in such instances, the term “patient” may refer to the broader population, and not to an individual.

The term “supratherapeutic dose,” as used herein, refers to a dose of apomorphine or a pharmaceutically acceptable salt thereof exceeding the therapeutic dose by at least 5 mg of the active agent.

The term “sustained ‘ON’ response,” as used herein, refers to a full “ON” response that gives a consistent benefit over a period of time (e.g., for at least 30 min, at least 45 min, at least 60 min, greater than 60 min, at least 70 min, at least 80 min, at least 90 min, etc.). A “full ‘ON’ response” refers to a period of time where medication is providing benefit with regard to mobility, stiffness and slowness and where the patient had adequate motor function to perform normal daily activities as assessed by the patient and/or as assessed by the clinician. An “OFF” episode refers to a period or event upon which an individual with Parkinson's disease experiences a loss of motor function, for example when the individual experiences a negative fluctuation in the effectiveness of a levodopa regimen.

The term “therapeutic dose,” as used herein, refers to a minimally effective dose of apomorphine or a pharmaceutically acceptable salt thereof determined by up-titration using techniques known in the art, e.g., as described in US 2018/0133146. Typically, a therapeutic dose is a quantity of apomorphine or a pharmaceutically acceptable salt thereof administered to a patient at once so as to produce at least one of the following effects: (1) a plasma concentration of at least 2.64 ng/mL of apomorphine in the patient within 45 minutes (e.g., within 30 minutes) of the administration; and (2) the patient in an “on” state within 45 minutes (e.g., within 30 minutes) of administering the administration. Non-limiting examples of therapeutic doses of apomorphine or a pharmaceutically acceptable salt thereof are 10 mg, 15 mg, 20 mg, 25 mg, 30 mg, 35 mg, 40 mg, and 45 mg (e.g., of apomorphine hydrochloride).

The term “Tmax,” as used herein, refers to an average observed time to the maximum plasma concentration produced in a group of patients (e.g., 10 or more) receiving an apomorphine film in an amount sufficient to produce an “on” state, where the amount of the film administered for each individual patient is the lowest therapeutic dose administered during up-titration of the individual patient (i.e., the Tmax accounting for variations in bioavailability) for a given route of administration (e.g., to oral mucosa, such as sublingual).

The terms “treatment,” “treat,” and “treating” as used herein in reference to Parkinson's disease or a related symptom (e.g., an “OFF” state) in a patient, is intended to refer to obtaining beneficial or desired results, e.g., clinical results, in a patient by administering a sublingual film to the patient. Beneficial or desired results may include alleviation or amelioration of one or more symptoms of Parkinson's disease (e.g., switching a patient “ON” from an “OFF” state, as assessed, e.g., in accordance with MDS-UPDRS); prevention of the occurrence of one or more symptoms of a dopamine-mediated disease or condition (e.g., Parkinson's disease) (e.g., prevention of an “OFF” state). In some embodiments, “treatment,” “treat” and “treating” includes one or more of the following: (a) inhibiting Parkinson's disease (for example, decreasing one or more symptoms resulting from the disease or condition and/or diminishing the extent of the disease or condition); (b) slowing or arresting the development of one or more symptoms associated with Parkinson's disease (for example, stabilizing the disease or condition and/or delaying the worsening of progression of the disease or condition); and/or (c) relieving Parkinson's disease (for example, causing the regression of clinical symptoms, ameliorating the disease or condition, delaying the progression of Parkinson's disease, and/or increasing quality of life.)

The present disclosure provides a method of treating a patient having Parkinson's disease, such as by improving motor function in a patient having an “OFF” episode associated with Parkinson's disease. In some embodiments, the method includes administering to the oral mucosa, e.g., buccally or sublingually, to the patient a supratherapeutic dose of apomorphine or a pharmaceutically acceptable salt thereof.

Such administration of the supratherapeutic dose of apomorphine or a pharmaceutically acceptable salt thereof may improve motor function of the patient. The motor function of the patient is typically assessed using a Movement Disorder Society Unified Parkinson's Disease Rating Scale Part III Motor Examination (MDS-UPDRS Part III) score. For example, the methods described herein may improve motor function in the patient, as measured by the MDS-UPDRS Part III score after a period of 30 to 90 minutes following the administering step. The improvement may be assessed relative to the MDS-UPDRS Part III score measured prior to the administering step. For example, the improvement may be, e.g., at least a 10% (e.g., at least 15%, at least 20%, at least 25%, at least 30%, at least 35%, at least 40%, at least 45%, or at least 50%; e.g., up to 80%, up to 70%, or up to 60%) reduction in the MDS-UPDRS Part III score relative to the MDS-UPDRS III score measured prior to the administering step

The improvement in the motor function of the patient after administration of the supratherapeutic dose of apomorphine or a pharmaceutically acceptable salt thereof may be superior to the improvement in the motor function of the patient after administration of a therapeutic dose of apomorpohine or a pharmaceutically acceptable salt thereof. Thus, the methods described herein may improve motor function of the patient, as measured by an improvement in a Movement Disorder Society Unified Parkinson's Disease Rating Scale Part III Motor Examination (MDS-UPDRS Part III) score in the patient after a period of 30 to 90 minutes following the administering step, where the improvement is greater than an improvement in the MDS-UPDRS Part III score after the same period following administration of a therapeutic dose of apomorphine or a pharmaceutically acceptable salt thereof to the patient. For example, the MDS-UPDRS Part III in the patient after a period of 30 to 90 minutes following the administering step may be, e.g., at least 1% (e.g., at least 2%, at least 3%, at least 4%, or at least 5%; e.g., up to 20%, up to 15%, or up to 10%) lower than the MDS-UPDRS Part III after the same period following administration of the therapeutic dose of apomorphine or a pharmaceutically acceptable salt thereof to the patient.

The supratherapeutic dose may include, e.g., sublingual administration of a dosage from containing from about 5 mg to 20 mg more apomorphine or a pharmaceutically acceptable salt thereof, than a therapeutic dose determined by up-titration. For example, the supratherapeutic dose may include a dosage strength which is, e.g., 5 mg, 10 mg, 15, mg, or 20 mg more of apomorphine or a pharmaceutically acceptable salt thereof than the therapeutic dose. The supratherapeutic dose may include a dosage strength of from about 15 mg to about 60 mg of apomorphine or a pharmaceutically acceptable salt thereof. For example, the supratherapeutic dose may be a dosage strength which is, e.g., 15 mg, 20 mg, 25 mg, 30 mg, 35 mg, 40 mg, 50 mg, or 60 mg of apomorphine or a pharmaceutically acceptable salt thereof.

The therapeutic dose is typically established by up-titrating the patient, or having the patient up-titrated, to determine a minimal dose that is therapeutically effective. This may be the minimal dose at which the patient achieves a full “ON” response. The therapeutic dose may include a dosage strength of from about 5 mg to about 35 mg of apomorphine or a pharmaceutically acceptable salt thereof. For example, the therapeutic dosage strength may be, e.g., 5 mg, 10 mg, 15 mg, 20 mg, 25, mg, 30 mg, or 35 mg of apomorphine or a pharmaceutically acceptable salt thereof.

Administration of the supratherapeutic dose may produce a full ON state in the patient. A full ON state is typically determined using MDS-UPDRS Part III score. Administration of the supratherapeutic dose may produce a sustained ON state in the patient. Administration of the supratherapeutic dose may produce a sustained ON response in the patient that is equivalent (or comparable) in duration to a sustained ON response from a dose of levodopa

Administration of the supratherapeutic dose may be in the form of a daily apomorphine therapy. The methods may comprise a daily apomorphine therapy comprising administering to the patient two or more (e.g., 3 or more, 4 or more, 5 or more, or 6 or more) supratherapeutic doses per day of the apomorphine or pharmaceutically acceptable salt thereof. In some embodiments, levodopa is not administered to the patient during the daily apomorphine therapy. In some embodiments, the apomorphine therapy is a monotherapy. In other embodiments the apomorphine is a combination therapy (e.g., with carbidopa and/or entacapone). In some embodiments, administration of a supratherapeutic dose as part of a daily apomorphine therapy may be able to replace a levodopa therapy. In some embodiments, the supratherapeutic dose may have both fast onset and sustained therapeutic effect.

Daily apomorphine therapy may be particularly suitable, and indeed may be able to replace a levodopa regimen in certain patients. In particular, patients that respond therapeutically to lower doses of apomorphine may be able to use supratherapeutic doses of apomorphine or a pharmaceutically acceptable salt thereof as a daily therapy to replace levodopa therapy. For example, patients that achieve a therapeutic dose (e.g., achieve a full “ON” state) at a sublingually-administered dose of apomorphine or a pharmaceutically acceptable salt thereof dosage strength of 20 mg or less, 15 mg or less, or 10 mg or less (e.g., 5 mg, 10 mg, or 15 mg) may be particularly suited to daily apomorphine therapy administered, e.g., to the oral mucosa, e.g. sublingually, in place of a levodopa regimen. The methods may comprise identifying a patient, or having a patient identified, as a candidate for apomorphine therapy when the, e.g., sublingual therapeutic dose for that patient is 35 mg or less, 30 mg or less, 25 mg or less, 20 mg or less, 15 mg or less, or 10 mg or less (e.g., 5 mg, 10 mg, or 15 mg). The methods may comprise selecting a patient, or having a patient selected, for apomorphine therapy when the therapeutic dose for that patient is 35 mg or less, 30 mg or less, 25 mg or less, 20 mg or less, 15 mg or less, or 10 mg or less (e.g., 5 mg, 10 mg, or 15 mg). Some examples of a sublingually-administered therapeutic dose and corresponding sublingually-administered supratherapeutic doses are shown below.

| Therapeutic Dose | Supratherapeutic Dose | | | |
|---|---|---|---|---|
| 5 mg | 10 mg | 15 mg | 20 mg | 25 mg |
| 10 mg | 15 mg | 20 mg | 25 mg | 30 mg |
| 15 mg | 20 mg | 25 mg | 30 mg | 35 mg |
| 20 mg | 25 mg | 30 mg | 35 mg | 40 mg |
| 25 mg | 30 mg | 35 mg | 40 mg | 45 mg |
| 30 mg | 35 mg | 40 mg | 45 mg | 50 mg |

In the methods described herein, apomorphine or a pharmaceutically acceptable salt thereof is typically administered to the patient sublingually. Sublingual administration of apomorphine or a pharmaceutically acceptable salt thereof may advantageously avoid first-pass metabolism-related reduction in the bioavailability of apomorphine.

Apomorphine or a pharmaceutically acceptable salt thereof may be administered to the patient in a unit dosage form, as described herein. For example, the unit dosage form may be, e.g., a lozenge, a pill, a tablet, a film, or a capsule. In some embodiments, the unit dosage form is a film. In some embodiments, the unit dosage form comprises a first portion comprising a pharmaceutically acceptable salt of apomorphine and a second portion comprising a pH neutralizing agent (e.g., pyridoxine). In some embodiments, the first portion comprises apomorphine particles comprising a pharmaceutically acceptable salt of apomorphine. In some embodiments, the pharmaceutically acceptable salt of apomorphine is an acid addition salt of apomorphine. In some embodiments, the acid addition salt of apomorphine is apomorphine hydrochloride.

Also disclosed herein is a pharmaceutical composition for administration to the oral mucosa, e.g. sublingual administration, comprising a supratherapeutic dose of apomorphine or a pharmaceutically acceptable salt thereof. The composition may be for use in treating Parkinson's disease, e.g., by improving motor function and by treating “OFF” episodes associated with Parkinson's disease.

### Methods of Treatment

The present disclosure provides methods of treating a patient having Parkinson's disease, such as by improving motor function in a patient experiencing an “OFF” episode associated with Parkinson's disease. The methods may involve, e.g., administering to oral mucosa of a patient (e.g., sublingually) an apomorphine film having a first portion including particles containing apomorphine or an acid addition salt thereof and a second portion containing a pH neutralizing agent. Prior to administering the film, the patient may have been identified as having low uptake; medium uptake; or high uptake of apomorphine via oral mucosa (e.g., sublingual uptake). The film administered in accordance with the methods described herein can be selected from a plurality of predetermined doses of varying strength having sufficient apomorphine content to produce at least the minimum effective concentration of apomorphine (i.e., at least 2.64 ng/mL). All embodiments of the pharmaceutical unit dosage forms described herein can be used in accordance with the methods described herein.

### Pharmacokinetics/Pharmacodynamics

The minimum effective concentration of apomorphine can be achieved within 30 minutes of administering the oral apomorphine film to the patient in accordance with some methods described herein. Preferably, an apomorphine Cₘₐₓ of less than 30 ng/mL (e.g., less than 20 ng/mL, less than 10 ng/mL, less than 7 ng/mL or less than 5 ng/mL) is produced after administering the film in accordance with the methods described herein. For example, apomorphine Cₘₐₓ may be in the range from 2.64 ng/mL to 30 ng/mL (e.g., from 2.64 ng/mL to 20 ng/mL, from 2.64 ng/mL to 10 ng/mL, or from 2.64 ng/mL to 5 ng/mL). Preferably, Tmax for apomorphine films administered in accordance with the methods described herein is in the range of from 10 minutes to 1 hour (e.g., from 20 minutes to 1 hour, or from 20 minutes to 50 minutes).

The patient treated in accordance with the methods described herein may be administered a supratherapeutic dose, which is at least 5 mg greater than the therapeutic dose. The therapeutic dose may be determined using methods known in the art, e.g., up-titration.

Up-titration may involve administering to a patient experiencing a symptom, e.g., an “Off” state, via the patients oral mucosa, e.g., sublingually, a first predetermined dosage of apomorphine or a pharmaceutically acceptable salt thereof (e.g., 10 mg of apomorphine or a pharmaceutically acceptable salt thereof), and determining if the dose administered was a therapeutic dose on the basis of symptom abatement, e.g., the patient exhibits a full “ON” response. If the amount of apomorphine or a pharmaceutically acceptable salt thereof is determined to be not a therapeutic dose, at a subsequent occurrence of the symptom a second predetermined dosage of apomorphine or a pharmaceutically acceptable salt thereof (e.g., 15.0 mg of apomorphine or a pharmaceutically acceptable salt thereof) is administered to the patient, and it is determined if the dose administered was a therapeutic dose of apomorphine or a pharmaceutically acceptable salt thereof. If the amount of apomorphine or a pharmaceutically acceptable salt thereof administered with the second predetermined dosage is determined to not be a therapeutic dose, at a subsequent occurrence of a symptom, a third predetermined dosage of apomorphine or a pharmaceutically acceptable salt thereof (e.g., 20.0±5.0 mg of apomorphine or a pharmaceutically acceptable salt thereof) is administered to the patient, and it is determined if a therapeutic dose of apomorphine or a pharmaceutically acceptable salt thereof was administered with the third predetermined dosage. If any of the administered amounts is found to be a therapeutic dose, the supratherapeutic dose has at least 5 mg (e.g., 5 mg, 10 mg, 15 mg, or 20 mg) of apomorphine or a pharmaceutically acceptable salt thereof more than the therapeutic dose. One of skill in the art will recognize, that third predetermined dosage contains more acid addition salt of apomorphine than the second predetermined dosage, which contains more acid addition salt of apomorphine than the first predetermined dosage. The up-titration thus may proceed by incrementally increasing the amount of apomorphine or a pharmaceutically acceptable salt thereof to be administered. The determination if a therapeutic dose of apomorphine was administered in any one of the above up-titration steps can be executed in accordance with methods known in the art, e.g., by evaluating UPDRS (e.g., UPDRS Part III) for the patient within a predetermined period (e.g., 30 minutes to 90 minutes) after administering apomorphine.

### Pharmaceutical Unit Dosage Forms

Pharmaceutical unit dosage forms described herein may be, e.g., films containing a first portion and a second portion. The films can be flexible. A pharmaceutical unit dosage form described herein may contain an acid addition salt of apomorphine and pharmaceutically acceptable excipients, such as a pharmaceutically acceptable polymer, a permeation enhancer, a hydrolyzed starch, an antioxidant, a plasticizing agent, a flavoring agent, and a coloring agent. References to a single pharmaceutically acceptable excipient include mixtures of pharmaceutically acceptable excipients within the scope of the recited type.

The portions in the pharmaceutical unit dosage forms can be domains or layers. In some embodiments, the portions are layers. In certain pharmaceutical unit dosage forms described herein, the first portion may be free of the pH neutralizing agent to prevent premature neutralization of the acid addition salt of apomorphine, thereby enhancing the film shelf-life by preventing oxidative degradation of neutralized apomorphine.

### Pharmaceutically Acceptable Polymers

The pharmaceutically acceptable polymers can be used to control toughness of the pharmaceutical unit dosage form described herein. In particular, pharmaceutical unit dosage forms described herein containing at least 20% (w/w) of a pharmaceutically acceptable high molecular weight polymer having a weight average molecular weight (Mw) that is greater than or equal to 60 kDa can exhibit a desirable degree of toughness (e.g., at least 100 g×mm or at least 150 g×mm). The pharmaceutical unit dosage form can contain 20% (w/w) or more (e.g., from 20% (w/w) to 50% (w/w), from 20% (w/w) to 40% (w/w), or from 20% (w/w) to 30% (w/w)) of a pharmaceutically acceptable high molecular weight polymer having a weight average molecular weight (M_{w}) that is greater than or equal to 60 kDa (e.g., from 60 kDa to 1,000 kDa).

The first portion may contain a pharmaceutically acceptable high molecular weight polymer having a weight average molecular weight (M_{w}) of 60 kDa or greater (e.g., from 60 kDa to 1,000 kDa). The second portion may contain a pharmaceutically acceptable high molecular weight polymer having a weight average molecular weight (M_{w}) of 60 kDa or greater (e.g., from 60 kDa to 1,000 kDa). In some embodiments, the pharmaceutically acceptable high molecular weight polymer has a weight average molecular weight (M_{w}) from 60 kDa to 500 kDa.

The first portion may contain a pharmaceutically acceptable low molecular weight polymer having a weight average molecular weight (M_{w}) less than 60 kDa (e.g., from 5 kDa to 50 kDa). The second portion may contain a pharmaceutically acceptable low molecular weight polymer having a weight average molecular weight (M_{w}) less than 60 kDa (e.g., from 5 kDa to 50 kDa). In certain unit dosage forms described herein, the second portion is free of an added polymer having a weight average molecular weight less than 60 kDa (e.g., a pharmaceutically acceptable cellulose derivative having a weight average molecular weight less than 60 kDa). The pharmaceutical unit dosage form described herein may contain less than 5% (w/w) of a pharmaceutically acceptable low molecular weight polymer (e.g., from 0.01% (w/w) to 5% (w/w), from 0.1% (w/w) to 4% (w/w), or from 1% (w/w) to 3% (w/w)).

Each pharmaceutically acceptable polymer can be independently carboxymethylcellulose, hydroxypropyl cellulose (HPC, such as Nisso HPC SSL, Nippon Soda Co., Ltd., Japan), hydroxypropyl methyl cellulose (also known as hypromellose or HPMC; commercially available under the tradename Methocel™ from Dow Chemical Company, Midland, Mich.), hydroxyethyl cellulose (HEC, commercially available from Hercules Incorporated, Aqualon Division under the tradename NATROSOL™), or methyl cellulose (such as Methocel™, Dow Chemical Company, Midland, Mich.), or a combination thereof.

### Plasticizing Agents

The pharmaceutical unit dosage forms described herein can include a plasticizing agent. Plasticizers will generally modify the feel, softness, flexibility (in an un-wetted state) of the unit dosage forms described herein. Examples of plasticizers include, without limitation, glycerol, propylene glycol, fatty acid esters, such as glyceryl oleate, polyalcohols, sorbitan esters, citric acid esters, polyethylene glycol (e.g., PEG 400), polyvinyl alcohol, polyvinyl methyl ether, triacetin; mannitol, xylitol, and sorbitol. In some embodiments, the plasticizing agent is glycerol. A pharmaceutical unit dosage form described herein can contain a plasticizing agent in the amount greater than 0% (w/w) and less than or equal to 8.5% (w/w) (e.g., in the range from 4% (w/w) to 8% (w/w)). In some embodiments, the pharmaceutical unit dosage form contains less than 5% (w/w) of a plasticizing agent (e.g., from 4% (w/w) to 5% (w/w) of a plasticizing agent). By including less than 8.5% (w/w) of a plasticizing agent in the pharmaceutical unit dosage form, the toughness of the pharmaceutical unit dosage forms described herein is improved. However, some plasticizing agent can be present for flexibility of the pharmaceutical unit dosage form described herein.

### Permeation Enhancers

Pharmaceutical unit dosage forms described herein can contain a permeation enhancer. For example, in some pharmaceutical unit dosage forms described herein, the second portion contains a permeation enhancer. In certain pharmaceutical unit dosage forms described herein, the first portion may be free of a permeation enhancer. The pharmaceutical unit dosage form described herein can contain less than 10% (w/w) (e.g., from 0.001% (w/w) to 10% (w/w)) of a permeation enhancer.

Permeation enhancers can be used to improve the permeability of the dopamine agonist at the mucosal membrane in the unit dosage forms described herein. One or more permeation enhancers maybe used to modulate the rate of mucosal absorption of the dopamine agonist. Any effective permeation enhancers may be used including, for example, ionic surfactants, nonionic surfactants, bile salts, such as sodium cholate, sodium glycocholate, sodium glycodeoxycholate, taurodeoxycholate, sodium deoxycholate, sodium lithocholate chenocholate, chenodeoxycholate, ursocholate, ursodeoxy-cholate, hyodeoxycholate, dehydrocholate, glycochenocholate, taurochenocholate, and taurochenodeoxycholate; sodium dodecyl sulfate (SDS), dimethyl sulfoxide (DMSO), N-lauroyl sacrcosine, sorbitan monolaurate, stearyl methacrylate, N-dodecylazacycloheptan-2-one, N-dodecyl-2-pyrrolidinone, N-dodecyl-2-piperidinone, 2-(1-nonyl)-1,3-dioxolane, N-(2-methoxymethyl) dodecylamine, N-dodecylethanolamine, N-dodecyl-N-(2-methoxymethyl)acetamide, 1-N-dodecyl-2-pyrrolidone-5-carboxylic acid, 2-pentyl-2-oxo-pyrrolidineacetic acid, 2-dodecyl-2-oxo-1-pyrrolidineacetic acid, 2-dodecyl-2-oxo-1-pyrrolidineacetic acid, 1-azacylioheptan-2-one-dodecylacetic acid, menthol, propylene glycol, glycerol monostearate, sorbitol monolaurate, glycerol dilaurate, tocopherol acetate, phosphatidyl choline, glycerol, polyethyleneglycol, monoglycerides, such as glycerol monostearate, glycerol monoloaurate, glycerol caprylate, diglycerides, triglycerides, and succinylated diglycerides and monoglycerides, such as glycerol succinyl caprylate lecithin, tween surfactants, sorbitan surfactants, sodium lauryl sulfate; salts, acids and other derivatives of saturated and unsaturated fatty acids, fatty alcohols, surfactants, bile salt analogs, derivatives of bile salts, or such synthetic permeation enhancers as described in U.S. Pat. No. 4,746,508, which is incorporated herein by reference.

### pH Neutralizing Agents

The pH neutralizing agent can be, for example, a film formed from a basic polymer. Polyamines which can be used in the unit dosage forms described herein include homo and copolymers of dimethylaminoethyl-acrylate, dimethylaminoethyl-methacrylate, dimethyl aminopropyl-acrylate, dimethylaminpropyl-methacrylate, or other similar amino-functionalized acrylate, chitosan or partially hydrolyzed chitin in a substantially basic form, homo and co polymers of polyethyleimine, polylysine, polyvinylimidazole, or polyvinylamine. In certain embodiments the polyamine is Eudragit E100.

Alternatively, the pH neutralizing agent can be a non-polymeric additive incorporated into a unit dosage form described herein. The pH neutralizing agent can be an inorganic base (e.g., aluminum hydroxide, aluminosilicates, calcium hydroxide, magnesium hydroxide, potassium hydroxide, sodium hydroxide, calcium carbonate, iron carbonate, magnesium carbonate, zinc carbonate, sodium carbonate, potassium carbonate, sodium bicarbonate, potassium bicarbonate, sodium phosphate monobasic, sodium phosphate dibasic, sodium phosphate tribasic, potassium phosphate monobasic, potassium phosphate dibasic, potassium phosphate tribasic, or a mixture thereof). The pH neutralizing agent can be an organic base (e.g., acetate salts, citrate salts, stearate salts, laurate salts, proprionate salts, lactate salts, succinate salts, oxalate salts, tartrate salts, glycolate salts, galacturonate salts, glucuronate salts, alginate salts, sorbate salts, caprylate salts, carboxymethyl cellulose, polyacrylate; or amines, such as pyridoxine, meglumine, lysine, Eudragit E, diethanolamine, glycine, citrate, acetate, histidine, N-methyl glucamine, or tris(hydroxymethyl)aminomethane, or a mixture thereof). Desirably, the pH neutralizing agent has a pKa of from 2.5 to 9.5 (e.g., a pKa of 2±0.5, 2.5±1, 3±1.5, 4±2, 5±2, 6±2, 7±1, or a pKa of from 4.5 to 8.5). In certain embodiments, the pH neutralizing agent is an organic base having a pKa of 5±2. One of skill in the art will recognize that pKa values refer to pKa in water at room temperature. In other embodiments, the pH neutralizing agent is pyridoxine.

### Other Excipients

A sweetener, flavoring agent and/or odorant can be added to the unit dosage forms described herein to make them more palatable. At least one flavoring agent or odorant composition may be used. Any effective flavor or odor may be rendered. The flavoring agents may be natural, artificial, or a mixture thereof. The flavoring agent gives a flavor that is will help to reduce the undesirable taste of the active ingredient. In one embodiment, the flavoring agent may give the flavor of mint, menthol, honey lemon, orange, lemon lime, grape, cranberry, vanilla berry, bubble gum, or cherry. The flavoring agent can be natural or artificial sweetener, such as sucrose, Magnasweet™, sucralose, xylitol, sodium saccharin, cyclamate, aspartame, acesulfame, and salts thereof. In some embodiments, the sweetener is sucralose.

Acid addition salts of apomorphine may be susceptible to oxidative degradation; while their susceptibility is lower than that of apomorphine in neutral form, inclusion of preservatives (e.g., antioxidants) is desirable to prolong the shelf life of the pharmaceutical unit dosage form described herein. Antioxidants that can be used in the pharmaceutical unit dosage forms described herein can be selected from the group consisting of thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and succinic acid, malic acid, humic acid, bile acid, bile extracts, bilirubin, biliverdin, EDTA, EGTA, and DTPA and salts thereof), sodium metabisulfite, sodium thiosulfate, vitamins and vitamin derivatives (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof. The total amount of antioxidant included in the films can be from 0.001% to 3% (w/w). In certain embodiments, the antioxidant is EDTA or a salt thereof, or sodium metabisulfite, or a mixture thereof.

The films described herein can include from 1 to 50% (w/w) of one or more hydrolyzed starches. Various hydrolyzed starches may be utilized including maltrodextrins with a DE greater than 10 and dried glucose syrups which have a DE above 20. Suitable hydrolyzed starch products are commercially available from Grain Processing Corporation of Muscatine, Iowa under trademarks such as MALTRIN M200®, MALTRIN 180®, and MALTRIN 250®. MALTRIN M200® is a hydrolyzed starch product having a DE of 20, and MALTRIN 180® is a hydrolyzed starch product having a DE of 18. Dextrose equivalent (DE) is the relative sweetness of sugars, oligosaccharides, or blends compared to dextrose, both expressed as a percentage. For example, a maltodextrin with a DE of 10 would be 10% as sweet as dextrose (DE=100), while sucrose, with a DE of 120, would be 1.2 times as sweet as dextrose. For solutions made from starch, it is an estimate of the percentage reducing sugars present in the total starch product. The DE describes the degree of conversion of starch to dextrose: starch is close to 0, glucose/dextrose is 100 (percent), dextrins vary between 1 and 13, and maltodextrins vary between 3 and 20. The DE gives an indication of the average degree of polymerisation (DP) for starch sugars. The rule of thumb is DE×DP=120.

In certain embodiments, the various components (e.g., plasticizers, penetration enhancers, flavoring agents, antioxidants, odorants, coloring agents, particulate base, and dopamine agonist particles) included in the unit dosage forms described herein can be combined and incorporated into a first portion that is acidic and includes the acid addition salt of apomorphine, or combined and incorporated into a second portion that includes a pH neutralizing component, or the components may be divided between the two portions. In some instances it may be desirable to minimize interaction between the acidic portion of the unit dosage form and the basic portion of the unit dosage form by including a barrier between the two. In some pharmaceutical unit dosage forms, a barrier can be included between the first portion and the second portion. For example, when the portions are layers, the barrier can be a third layer interposed between the first portion (first layer) and the second portion (second layer). Alternatively, the barrier can be a rapidly dissolving coating on the surface of a particulate component in the unit dosage form, such as a coated particulate base coated onto, or embedded within, a first portion of the unit dosage form. In still another approach, the barrier can be a rapidly dissolving coating on the surface of apomorphine particles in the unit dosage form. These approaches can be utilized to ensure that the acid addition salt of apomorphine in the first portion of the unit dosage form is not neutralized prior to the administration to a patient.

### Apomorphine

Pharmaceutical unit dosage forms described herein include an acid addition salt of apomorphine. Examples of acids that may be used in acid addition salts of apomorphine include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids; polymeric acids such as tannic acid, carboxymethyl cellulose, or alginic acid; and inorganic acid such as hydrochloric acid, hydrobromic acid, sulfuric acid, or phosphoric acid. In some embodiments, the acid addition salt of apomorphine is apomorphine hydrochloride. A pharmaceutical unit dosage form described herein may contain from 2 mg to 60 mg of an acid addition salt of apomorphine (e.g., from 8 mg to 45 mg of an acid addition salt of apomorphine). Certain exemplary pharmaceutical unit dosage forms may contain 10.0±2.0 mg, 12.5±2.5 mg, 15.0±2.5 mg, 17.5±2.5 mg, 20.0±5.0 mg, 25.0±5.0 mg, 30.0±10.0 mg, 30.0±5.0 mg, 35.0±10.0 mg, or 35.0±5.0 mg of an acid addition salt of apomorphine. The first portion may contain from 20% (w/w) to 60% (w/w) of an acid addition salt of apomorphine (e.g., from 30% (w/w) to 60% (w/w) or from 40% (w/w) to 60% (w/w)) relative to the weight of the dry first portion. In particular, the film may contain 50±10% (w/w) (e.g., 54±10% (w/w)) or an acid addition salt of apomorphine. Despite high content of an acid addition salt of apomorphine, the pharmaceutical unit dosage forms described herein can exhibit toughness as described herein. The desirable toughness of the pharmaceutical unit dosage forms described herein can be achieved, e.g., as described herein.

The pharmaceutical compositions described herein can provide a rapid-dissolving, rapid absorption solid dosage form that includes apomorphine or a pharmaceutically acceptable salt thereof. Non-limiting examples of a rapid absorption solid dosage form include, for example, orally disintegrating tablets and orally disintegrating films. In some embodiments, the rapid absorption solid further comprises a pH neutralizing agent. Inclusion of a pH neutralizing agent may facilitate absorption of apomorphine.

A pharmaceutical composition disclosed herein may be provided as a unit dosage form that is a film comprising apomorphine or a pharmaceutically acceptable salt thereof. Apomorphine or a pharmaceutically acceptable salt thereof may be present in the films as a solid solution in a polymeric carrier. Films may furthermore undergo rapid dissolution or rapid disintegration in the oral media (e.g., saliva), e.g., within 10 minutes (e.g., within 5 minutes, within 3 minutes, within 2 minutes, or within 1 minute).

Provided herein are formulations comprising apomorphine or a pharmaceutically acceptable salt thereof, and one or more pharmaceutically acceptable excipients. Non-limiting examples of pharmaceutically acceptable excipients includes, any binder, filler, adjuvant, carrier, solubilizer, antioxidant, buffering agent, permeation enhancer, hydrolyzed starches, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, vehicle, disintegrants, or lubricant which has been approved by the United States Food and Drug Administration as being acceptable for use in humans or domestic animals.

The formulations described herein can include an antioxidant system (e.g., a combination of at least two antioxidants) in a unit dosage form described herein. Antioxidants are known in the art. Non-limiting examples of antioxidants that may be included in the dosage form are thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, or penta-, hexa- or heptathionine-sulphoximine), metal chelators (e.g, a-hydroxy-fatty acids, palmitic acid, phytic acid, lactoferrin, citric acid, lactic acid, succinic acid, malic acid, humic acid, bile acid, bile extracts, bilirubin, biliverdin, EDTA, EGTA, or DTPA or a salt thereof), metabisulfite salts (e.g., sodium metabisulfite or potassium metabisulfite), bisulfite salts (e.g., sodium bisulfite or potassium bisulfite), sodium thiosulfate, vitamins and vitamin derivatives (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, or trihydroxybutyrophenone), benzoates (e.g., coniferyl benzoate), cyclodextrins (e.g., P-cyclodextrin or sulfobutyl-P-cyclodextrin), uric acid, mannose, propyl gallate, selenium (e.g., selenium-methionine), stilbenes (e.g., stilbene oxide and trans-stilbene oxide), and combinations thereof.

The formulations described herein can include a permeation enhancer. The permeation enhancer may be, for example, an ionic surfactant, nonionic surfactant, polysorbate, derivatives of tocopherol, poloxamer, monoglyceride, diglyceride, fatty acid, fatty alcohol, or mixtures thereof. In some embodiments, the permeation enhancer is glycerol monostearate.

The pharmaceutical unit dosage forms described herein can include apomorphine microparticles having a D50 of from 1 μm to 500 μm (e.g., from 1 μm to 100 μm or 1 μm to 50 μm). The starting microparticles can be microspheres can be made of an acid addition salt of apomorphine and predominantly crystalline, predominantly microcrystalline, predominantly amorphous, or a mixture thereof. An acid addition salt of apomorphine can be encapsulated in the microsphere or included in a dissolved-drug microsphere.

In an alternative approach, the pharmaceutical formulations described herein can include apomorphine particles having an effective particle size of less than 1 μm (i.e., nanoparticulate formulations). The starting microparticles can be microspheres can be made of an acid addition salt of apomorphine and predominantly crystalline, predominantly microcrystalline, predominantly amorphous, or a mixture thereof. An acid addition salt of apomorphine can be encapsulated in the microsphere or included in a dissolved-drug microsphere.

These apomorphine particles can be made by using any method known in the art for achieving the desired particle sizes. Useful methods include, for example, milling, homogenization, supercritical fluid fracture, or precipitation techniques. Exemplary methods are described in U.S. Pat. Nos. 4,540,602; 5,145,684; 5,518,187; 5,718,388; 5,862,999; 5,665,331; 5,662,883; 5,560,932; 5,543,133; 5,534,270; and 5,510,118; 5,470,583, each of which is specifically incorporated by reference.

Also disclosed herein is a method of treating Parkinson's disease in a patient by administering to the patient a therapeutically effective amount of apomorphine or a pharmaceutically acceptable salt thereof. In some embodiments, administration of apomorphine to a patient is via the oral mucosa. In some embodiments, administration of apomorphine to a patient is by sublingual administration. In some embodiments, administration of apomorphine to a patient is by buccal administration. Apomorphine or a pharmaceutically acceptable salt thereof may be administered as a monotherapy. Alternatively, apomorphine or a pharmaceutically acceptable salt thereof may be administered in a combination therapy (e.g., in a combination therapy with carbidopa and/or entacapone).

Advantageously, apomorphine or a pharmaceutically acceptable salt thereof may be used to supplant the first daily dose of levodopa (e.g., apomorphine or a pharmaceutically acceptable salt thereof may be administered to the patient in the morning at the time and instead of the first daily dose of levodopa). Alternatively, apomorphine or a pharmaceutically acceptable salt thereof may be used to augment the first daily dose of levodopa (e.g., apomorphine or a pharmaceutically acceptable salt thereof may be administered to the patient in the morning at or around the time of the first daily dose of levodopa). In some embodiments, apomorphine therapy may be employed instead of levodopa therapy.

In the methods described herein, when apomorphine or a pharmaceutically acceptable salt thereof is administered as part of a combination therapy with levodopa, the first levodopa dose of the day is supplanted or augmented with a therapeutically effective amount of apomorphine or a pharmaceutically acceptable salt thereof. For example, the patient may be receiving, e.g., a levodopa regimen, and the administering step supplants or augments the first levodopa dose of the day. Levodopa regimens (typically, orally administered levodopa regimens) are known in the art. See, e.g., prescribing information for SINEMET and RYTARY. For example, a levodopa regimen may comprise two or more, three or more, four or more, five or more, or six or more daily doses of levodopa throughout the day.

The methods described herein may further comprise administering the second and further levodopa doses of the day after administering the therapeutically effective amount of apomorphine or a pharmaceutically acceptable salt thereof.

In some embodiments, the combination therapy excludes levodopa.

For administration to the oral mucosa, the therapeutically effective amount, as determined by methods described herein, may be, e.g., 10 mg to 60 mg of apomorphine or a pharmaceutically acceptable salt thereof. For example, the therapeutically effective amount administered to the oral mucosa may be, e.g., 10 mg, 15 mg, 20 mg, 25 mg, 30 mg, 35 mg, 40 mg, 50 mg, or 60 mg of apomorphine or a pharmaceutically acceptable salt thereof.

In the methods described herein, apomorphine or a pharmaceutically acceptable salt thereof is typically administered to the patient sublingually. Sublingual administration of apomorphine or a pharmaceutically acceptable salt thereof may advantageously avoid first-pass metabolism-related reduction in the bioavailability of apomorphine.

Apomorphine or a pharmaceutically acceptable salt thereof may be administered to the patient in a unit dosage form, as described herein. For example, the unit dosage form may be, e.g., a lozenge, a pill, a tablet, a film, or a capsule. In some embodiments, the unit dosage form is a film. In some embodiments, the unit dosage form comprises a first portion comprising a pharmaceutically acceptable salt of apomorphine and a second portion comprising a pH neutralizing agent (e.g., pyridoxine). In some embodiments, the first portion comprises apomorphine particles comprising a pharmaceutically acceptable salt of apomorphine. In some embodiments, the pharmaceutically acceptable salt of apomorphine is an acid addition salt of apomorphine. In some embodiments, the acid addition salt of apomorphine is apomorphine hydrochloride.

The supplanting or augmenting dose may be a therapeutic dose or a supratherapeutic dose. In some embodiments, the supplanting dose is a supratherapeutic dose. In some embodiments, the supratherapeutic dose may have both fast onset and sustained therapeutic effect. In some embodiments, selecting a patient population which experiences a Full “ON” state with sublingual apomorphine dosage strengths of 20 mg or less may permit treatment without concomitant levodopa administration when this patient population is provided supratherapeutic doses of apomorphine as described herein.

The supplanting dose may achieve a sustained “ON” response such that the patient has adequate motor function until they take a dose of levodopa later in the day, e.g., 90 minutes, 2 hours, 3 hours, 4 hours, or more after administration of the supplanting dose. Thus, in some methods with a supplanting dose, levodopa is not administered to the patient for at least 90 minutes, at least 2 hours, at least 3 hours, or at least 4 hours following administration of the apomorphine or pharmaceutically acceptable salt thereof.

The augmenting dose may be administered concomitantly with the first levodopa dose of the day. Alternatively, the augmenting dose may be administered before the first levodopa dose of the day, e.g., from about 15 to about 60 minutes before the first levodopa dose of the day.

In some embodiments, the augmenting dose is a therapeutic dose. The patient receiving the augmenting dose may only achieve a therapeutic dose at higher dosages (e.g., 20 mg, 25 mg, 30 mg, or 35 mg) and therefore may not be suited for a supratherapeutic dose. As such, the patient may not achieve a sustained “ON” response from apomorphine alone and may be best suited to use apomorphine or a pharmaceutically acceptable salt to augment a dose of levodopa. In some embodiments, the augmenting dose of apomorphine or a pharmaceutically acceptable salt thereof may have faster onset than levodopa.

The present methods may also comprise determining whether a patient should be treated with (1) apomorphine therapy in the absence of levodopa, (2) a single daily dose of apomorphine (or salt thereof) to supplant the first dose of the day of levodopa, or (3) a single daily dose of apomorphine (or salt thereof) to augment the first dose of the day of levodopa. A patient may be selected for apomorphine therapy in the absence of levodopa if the patient responds therapeutically to a low dose of apomorphine or a pharmaceutically acceptable salt thereof. A patient may also be selected for a single daily dose of apomorphine (or salt thereof) to supplant the first dose of the day of levodopa if the patient responds therapeutically to a low dose of apomorphine or a pharmaceutically acceptable salt thereof. And a patient may be selected for a single daily dose of apomorphine (or salt thereof) to augment the first dose of the day of levodopa if the patient does not respond therapeutically to a low dose of apomorphine or a pharmaceutically acceptable salt thereof. For example, a patient that achieves a full “ON” response at a dose of 10 mg may be administered apomorphine therapy in the absence of levodopa or a single daily dose of apomorphine (or salt thereof) to supplant the first dose of the day of levodopa. A patient that does not achieve a full “ON” response at a dose of 10 mg but achieves a full “ON” response after up-titration to a higher dose (e.g., 25 mg) may be administered a single daily dose of apomorphine (or salt thereof) to augment the first dose of the day of levodopa.

Also disclosed herein is a pharmaceutical composition comprising apomorphine or a pharmaceutically acceptable salt thereof. The composition may be for use in treating Parkinson's disease, e.g., by improving motor function and by treating “OFF” episodes associated with Parkinson's disease. The composition may be for use to supplant or augment the first dose of the day of levodopa.

In some embodiments, provided is a method of treating a patient having Parkinson's disease without causing a clinically significant adverse event associated with subcutaneous administration of apomorphine, the method comprising sublingually administering a pharmaceutical composition comprising from about 10 mg to about 60 mg of apomorphine, or a pharmaceutically acceptable salt thereof, to the patient.

In some embodiments, provided is a method of treating Parkinson's disease, in a patient in need thereof, without causing a clinically significant adverse event associated with subcutaneous administration of apomorphine, the method comprising sublingually administering a pharmaceutical composition comprising from about 10 mg to about 60 mg of apomorphine, or a pharmaceutically acceptable salt thereof, to the patient.

In some embodiments, provided is a method of administering at least 10 mg of apomorphine to a patient having Parkinson's disease without causing a clinically significant adverse event associated with subcutaneous administration of apomorphine, the method comprising sublingually administering a pharmaceutical composition comprising at least 10 mg of apomorphine, or a pharmaceutically acceptable salt thereof, to the patient.

In some embodiments, provided is a method of minimizing the risk of one or more adverse events associated with subcutaneous administration of apomorphine in a patient having Parkinson's disease comprising sublingually administering a pharmaceutical composition comprising apomorphine, or a pharmaceutically acceptable salt thereof, to the patient.

In some embodiments, provided is a method of minimizing one or more adverse events associated with subcutaneous administration of apomorphine in a patient having Parkinson's disease comprising sublingually administering a pharmaceutical composition comprising apomorphine, or a pharmaceutically acceptable salt thereof, to the patient.

In some embodiments, provided is a method of treating a patient having Parkinson's disease comprising: (a) sublingually administering a pharmaceutical composition comprising at least 10 mg of apomorphine, or a pharmaceutically acceptable salt thereof; (b) monitoring, or having monitored, the patient's QT interval; and (c) discontinuing use of apomorphine, or a pharmaceutically acceptable salt thereof, if the QT interval is prolonged more than 10 ms.

In some embodiments, provided is a method of treating a patient having Parkinson's disease comprising: (a) selecting a sublingually administered pharmaceutical composition that does not clinically significantly increase QT interval prolongation, wherein the pharmaceutical composition comprises at least 10 mg of apomorphine, or a pharmaceutically acceptable salt thereof; (b) monitoring, or having monitored, the patient's QT interval; and (c) discontinuing use of the pharmaceutical composition if the patient demonstrates clinically significant QT interval prolongation.

In some embodiments, provided is a method of treating Parkinson's disease with a pharmaceutical composition that provides incidence of QT interval prolongation no greater than that in a placebo-treated group, comprising sublingually administering the pharmaceutical composition to a patient in need thereof, wherein the pharmaceutical composition comprises at least 10 mg of apomorphine, or a pharmaceutically acceptable salt thereof.

In some embodiments, provided is a method of treating Parkinson's disease with a pharmaceutical composition without inducing QT interval prolongation greater than that in a placebo-treated group, comprising sublingually administering the pharmaceutical composition to a patient in need thereof, wherein the pharmaceutical composition comprises at least 10 mg of apomorphine, or a pharmaceutically acceptable salt thereof.

In some embodiments, QT prolongation is characterized as one or both of: a QTcF interval in the patient of greater than 450 msec at any time point not present at baseline; and an increase in QTcF interval from baseline of greater than or equal to 30 msec for at least one post-baseline measurement.

In some embodiments, QT prolongation is characterized as one or both of: a QTcF interval in the patient of greater than 450 msec at any time point not present at baseline if the patient is a male or greater than 470 msec at any time point not present at baseline if the patient is a female; and an increase in QTcF interval from baseline of greater than or equal to 30 msec for at least one post-baseline measurement.

In some embodiments, QT prolongation is characterized as one or both of: a QTcF interval in the patient of greater than 450 msec at any time point not present at baseline if the patient is a male or greater than 470 msec at any time point not present at baseline if the patient is a female; and an increase in QTcF interval from baseline of greater than or equal to 60 msec for at least one post-baseline measurement.

Prolongation of the QTc interval of the electrocardiogram (ECG) may be associated with the development of torsade de pointes, a ventricular arrhythmia that can cause syncope and may progress to ventricular fibrillation and sudden death. The average QTc interval in healthy adults is approximately 400 msec. A QTc interval of 500 msec or greater is considered to be a substantial risk factor for torsade de pointes.

In some embodiments, provided is a method of treating a patient having Parkinson's disease with an apomorphine therapy, the apomorphine therapy comprising sublingually administering a pharmaceutical composition comprising from about 10 mg to about 60 mg of apomorphine, or a pharmaceutically acceptable salt thereof, to the patient, wherein the effect of the apomorphine therapy lasts more than 60 minutes post administration. In some embodiments, the apomorphine therapy is provided following an “OFF” episode of the patient.

In some embodiments, provided is a method of treating Parkinson's disease, in a patient in need thereof, the method comprising sublingually administering a pharmaceutical composition comprising from about 10 mg to about 60 mg of apomorphine, or a pharmaceutically acceptable salt thereof, to the patient, wherein the patient experiences a sustained “ON” response for at least 60 minutes post-administration.

In some embodiments, provided is a method of treating a patient having an “OFF” episode associated with Parkinson's disease by providing a sustained “ON” response for at least 60 minutes post-administration, the method comprising sublingually administering a pharmaceutical composition comprising from about 10 mg to about 60 mg of apomorphine, or a pharmaceutically acceptable salt thereof, to the patient.

In some embodiments, provided is a method of assessing the safety and responsiveness of a treatment for an “OFF” episode in a patient having Parkinson's disease, the method comprising sublingually administering, under medical supervision, to a treatment naïve patient (e.g., such as a patient at risk for hypotension or QT prolongation) a sublingual film comprising apomorphine particles comprising an acid addition salt of apomorphine and a second portion comprising a pH neutralizing agent. In some embodiments, the medical supervision comprises monitoring a vital sign of the subject, (e.g., blood pressure).

In some embodiments, provided is a method of treating a patient having an “OFF” episode associated with Parkinson's disease, the method comprising sublingually administering to the patient a first pharmaceutical composition comprising a first dose of apomorphine or a pharmaceutically acceptable salt thereof; and sublingually administering a second pharmaceutical composition comprising a second dose of apomorphine or a pharmaceutically acceptable salt thereof; wherein the first dose and the second dose are consecutive, and at least 2 hours separate administration of the first dose and the second dose. In some embodiments, the second dose is greater than the first dose if the patient does not respond to the first dose. In some embodiments, the first dose comprises about 10 mg of apomorphine or a pharmaceutically acceptable salt thereof. In some embodiments, if the first dose is ineffective, a second dose is not given for the “OFF” episode. In some embodiments, the first pharmaceutical composition is a sublingual film comprising a first portion comprising apomorphine particles comprising an acid addition salt of apomorphine, and a second portion comprising a pH neutralizing agent; and the second pharmaceutical composition is a sublingual film comprising a first portion comprising apomorphine particles comprising an acid addition salt of apomorphine, and a second portion comprising a pH neutralizing agent.

In some embodiments, provided herein is a method of treating a patient having an “OFF” episode associated with Parkinson's disease, the method comprising: (a) sublingually administering to the patient having a first “OFF” episode a first dose of a sublingual film comprising apomorphine particles comprising an acid addition salt of apomorphine and a second portion comprising a pH neutralizing agent; (b) if the patient does not respond to the first dose, sublingually administering to the patient having a subsequent “OFF” episode a subsequent dose of the sublingual film, wherein the subsequent dose comprises more of the acid addition salt of apomorphine than the first dose; (c) if the patient in step (b) does not respond, repeating step (b) such that each subsequent dose comprises more of the acid addition salt of apomorphine than the preceding dose until the patient responds to the treatment; and (d) sublingually administering to the patient a therapeutically effective dose of the sublingual film to the patient; wherein the dose identified in steps (a), (b), or (c) to which the patient responds is the therapeutically effective dose; wherein no more than one dose is administered per “OFF” episode in each of steps (a), (b), and (c); and wherein at least 2 hours separate administration of two consecutive doses of the sublingual films to the patient.

Apomorphine and pharmaceutically acceptable salts thereof are well known in the art. In some embodiments, the apomorphine or a pharmaceutically acceptable salt thereof (i.e., for use in the methods and compositions of the present disclosure) is an acid addition salt of apomorphine. In some embodiments, the apomorphine or a pharmaceutically acceptable salt thereof is apomorphine hydrochloride. For example, the apomorphine hydrochloride is apomorphine hydrochloride hemihydrate.

Apomorphine or a pharmaceutically acceptable salt thereof may be used in any suitable dosage in the methods and compositions described herein. In some embodiments, a pharmaceutical composition of the present disclosure comprises from about 10 mg to about 60 mg of apomorphine, or a pharmaceutically acceptable salt thereof (e.g., from about 10 mg to about 60 mg of apomorphine hydrochloride). In some embodiments, a pharmaceutical composition of the present disclosure comprises from about 10 mg to about 35 mg of apomorphine, or a pharmaceutically acceptable salt thereof (e.g., from about 10 mg to about 35 mg of apomorphine hydrochloride). For example, in some embodiments, the pharmaceutical composition comprises about 10 mg of apomorphine, or a pharmaceutically acceptable salt thereof (e.g., about 10 mg of apomorphine hydrochloride); in some embodiments, the pharmaceutical composition comprises about 15 mg of apomorphine, or a pharmaceutically acceptable salt thereof (e.g., about 15 mg of apomorphine hydrochloride); in some embodiments, the pharmaceutical composition comprises about 20 mg of apomorphine, or a pharmaceutically acceptable salt thereof (e.g., about 20 mg of apomorphine hydrochloride); in some embodiments, the pharmaceutical composition comprises about 25 mg of apomorphine, or a pharmaceutically acceptable salt thereof (e.g., about 25 mg of apomorphine hydrochloride); in some embodiments, the pharmaceutical composition comprises about 30 mg of apomorphine, or a pharmaceutically acceptable salt thereof (e.g., about 30 mg of apomorphine hydrochloride); and in some embodiments, the pharmaceutical composition comprises about 35 mg of apomorphine, or a pharmaceutically acceptable salt thereof (e.g., about 35 mg of apomorphine hydrochloride). The pharmaceutical composition may comprise a single sublingual film comprising the desired dosage or two or more sublingual films comprising the desired dosage (e.g., a 35 mg dosage may be achieved by administering 20 mg and 15 mg dosage sublingual films).

The patient of the present disclosure is a patient having Parkinson's disease. In some embodiments, the patient having Parkinson's disease is experiencing motor fluctuations prior to administration (e.g., immediately prior to administration). In some embodiments, the patient having Parkinson's disease is experiencing an “OFF” episode prior to administration (e.g., immediately prior to administration). In some embodiments, the patient having Parkinson's disease is experiencing end-of-dose wearing “OFF”, early morning “OFF,” partial “OFF,” delayed “OFF,” No-ON “OFF” or unpredictable “OFF” prior to administration (e.g., immediately prior to administration).

Disclosed is a method of treating Parkinson's disease in a subject (e.g., treating hypomobility or an “OFF” episode” in a subject having Parkinson's disease). The method involves administering to the subject apomorphine or a pharmaceutically acceptable salt thereof, or a pharmaceutical composition disclosed herein. In some embodiments, apomorphine or a pharmaceutically acceptable salt thereof is administered to the oral mucosa of a subject (e.g., sublingual administration or buccal administration). In some embodiments, apomorphine or a pharmaceutically acceptable salt thereof or a pharmaceutical composition disclosed herein is administered in a therapeutically effective amount, for example, to produce at least the minimum effective plasma concentration of apomorphine (i.e., at least 2.6 ng/ml). In some embodiments, no more than 5 doses per day of the pharmaceutical composition disclosed herein are administered to the subject. In some embodiments, the average frequency of dosing is 2 to 3 times per day. The pharmaceutical compositions described herein can be used in the methods described herein.

In some embodiments, the method of treating Parkinson's disease comprises treating acute or intermittent “OFF” episodes associated with Parkinson's disease. In some embodiments, the acute or intermittent “OFF” episodes associated with Parkinson's disease comprises at least one of end-of-dose wearing “OFF” (including early morning “OFF”), partial “OFF,” delayed “OFF,” No-ON “OFF,” or unpredictable “OFF.” In some embodiments, the “OFF” episode is end-of-dose wearing “OFF.” In some embodiments, the “OFF” episode is early morning “OFF.” In some embodiments, the “OFF” episode is partial “OFF.” In some embodiments, the “OFF” episode is delayed “OFF.” In some embodiments, the “OFF” episode is No-ON “OFF.” In some embodiments, the “OFF” episode is unpredictable “OFF.”

In some embodiments, no 5HT3 antagonist is administered concomitantly with the film. In some embodiments, no ondansetron, granisetron, dolasetron, palonosetron, or alosetron is administered concomitantly with the film. In some embodiments, the methods described herein further comprise administration of an antiemetic, such as trimethobenzamide.

The minimum effective concentration of apomorphine can be achieved within 30 minutes of administering apomorphine or a pharmaceutically acceptable salt thereof to the subject. Apomorphine or a pharmaceutically acceptable salt thereof may be administered in a pharmaceutical composition. In some embodiments, an apomorphine Cₘₐₓ of less than 30 ng/ml (e.g., less than 20 ng/ml, less than 10 ng/ml, less than 7 ng/ml or less than 5 ng/ml) is produced after the administering step. In some embodiments, apomorphine Cₘₐₓ may be in the range 2.6 ng/ml to 30 ng/ml (e.g., 2.6 ng/ml to 20 ng/ml, 2.6 ng/ml to 10 ng/ml, or 2.6 ng/ml to 5 ng/ml). In some embodiments, Tmax for observed for the methods of disclosed herein is in the range of 10 minutes to 1 hour (e.g., 20 minutes to 1 hour, or 20 minutes to 50 minutes). The identification of an appropriate dose for the subject may be performed using methods known in the art, e.g., titration. In some embodiments, titration is up-titration. Up-titration may involve administering to the subject a first predetermined dosage of apomorphine (e.g., 12.5±2.5 mg of apomorphine or a pharmaceutically acceptable salt thereof; 20.0±5.0 mg of apomorphine or a pharmaceutically acceptable salt thereof; or 30.0±5.0 mg of apomorphine or a pharmaceutically acceptable salt thereof), and determining if a therapeutically effective amount of apomorphine was administered. The determination of whether an effective amount of apomorphine was administered in any one of the above up-titration steps can be executed in accordance with methods known in the art, e.g., by evaluating UPDRS (e.g., UPDRS Part III) for the subject within a predetermined period (e.g., 30 minutes or 45 minutes) after administering apomorphine or by measuring apomorphine plasma concentration in a blood sample obtained from the subject within a predetermined period (e.g., 30 minutes or 45 minutes) after administering apomorphine.

In some embodiments, sublingual administration of the apomorphine, or a pharmaceutically acceptable salt thereof, produces a duration of effect of at least 60 minutes. For example, the duration of effect may be greater than 60 minutes, e.g., at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, or at least 120 minutes. In some embodiments, the duration of effect refers to the duration of a sustained “ON” response. Accordingly, in some embodiments, sublingual administration of the apomorphine, or a pharmaceutically acceptable salt thereof, produces a sustained “ON” response in the patient for at least 60 minutes post-administration (e.g., greater than 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, or at least 120 minutes).

The methods of the present disclosure have low incidence of adverse events associated with subcutaneous apomorphine. In some embodiments, the adverse event associated with subcutaneous administration of apomorphine is prolongation of the QT interval, i.e., the method has a low incidence of QT prolongation. In some embodiments, the mean change from baseline in QTcF in the patient is no greater than 10 ms. In some embodiments, the mean change from baseline in QTcF in the patient is no greater than 5 ms. In some embodiments, the mean change from baseline in QTcF in the patient is no greater than about 3.3 ms. In some embodiments, the mean change from baseline in QTcF in the patient 60 minutes after administration is not greater than about 3.3 ms. In some embodiments, the time-matched and placebo-adjusted mean change from baseline in QTcF in the patient is not greater than 10 ms. In some embodiments, the time-matched and placebo-adjusted mean change from baseline in QTcF in the patient is not greater than about 6.2 ms. In some embodiments, the time-matched and placebo-adjusted mean change from baseline in QTcF in the patient 60 minutes after administration is not greater than about 6.2 ms. In some embodiments, the patient does not experience a QTcF greater than 500 ms. In some embodiments, the patient does not experience a QTcF greater than 480 ms. In some embodiments, the patient does not have a clinically significant risk of experiencing a QTcF greater than 450 ms. In some embodiments, the patient does not experience an increase in QTcF greater than 60 ms. In some embodiments, the patient does not have a clinically significant risk of experiencing an increase in QTcF of greater than 30 ms.

In some embodiments, the patient has an elevated risk of QT prolongation from subcutaneous administration of apomorphine. In some embodiments, the patient has a history of QT prolongation from a prior therapy. In some embodiments, the patient is susceptible to QT prolongation from subcutaneous administration of apomorphine. In some embodiments, the patient has hypokalemia, Hepatitis C, HIV, T-wave abnormalities on electrocardiogram, is female, is geriatric, or is taking a second active agent known to increase risk of QT prolongation. In some embodiments, the patient is at risk for hypotension, e.g., orthostatic hypotension. In some embodiments, the patient has a history of hypotension, cardiovascular disease, or the patient is currently using antihypertensive medication.

In some embodiments, the patient is not actively monitored for QT prolongation. In some embodiments, the patient is not warned about QT prolongation. In some embodiments, the patient is not concurrently treated for QT prolongation.

In some embodiments, the adverse event associated with subcutaneous administration of apomorphine is selected from prolongation of the QT interval, orthostatic hypotension, syncope, dyskinesia, hallucinations, and impulse control problems, or any combination thereof. For example, in some embodiments, the adverse event associated with subcutaneous administration of apomorphine is prolongation of the QT interval. In some embodiments, the patient's risk of prolongation of the QT interval from the treatment is no greater than 2%. In some embodiments, the adverse event associated with subcutaneous administration of apomorphine is orthostatic hypotension. In some embodiments, the patient's risk of orthostatic hypotension from the treatment is no greater than 2%. In some embodiments, the adverse event associated with subcutaneous administration of apomorphine is syncope. In some embodiments, the patient's risk of syncope from the treatment is no greater than 2%. In some embodiments, the adverse event associated with subcutaneous administration of apomorphine is dyskinesia. In some embodiments, the patient's risk of dyskinesia from the treatment is no greater than 2%. In some embodiments, the adverse event associated with subcutaneous administration of apomorphine is hallucinations. In some embodiments, the patient's risk of hallucinations from the treatment is no greater than 2%. In some embodiments, the adverse event associated with subcutaneous administration of apomorphine is impulse control problems. In some embodiments, the patient's risk of impulse control problems from the treatment is no greater than 2%.

Methods of the present disclosure are efficacious for treating the patient having Parkinson's disease. In some embodiments, the methods are efficacious for treating “OFF” episodes of Parkinson's disease. In some embodiments, the method results in a reduction from baseline in MDS-UPDRS Part III motor examination score of at least 5 when measured 30 minutes post-dose. In some embodiments, the method results in a reduction from baseline in MDS-UPDRS Part III motor examination score of at least 10 when measured 30 minutes post-dose. In some embodiments, the method results in a reduction from baseline in MDS-UPDRS Part III motor examination score of at least about 11.1 when measured 30 minutes post-dose.

Also provided herein are pharmaceutical compositions and dosage forms for sublingual administration, comprising apomorphine or a pharmaceutically acceptable salt thereof, and one or more pharmaceutically acceptable excipients. Compositions and dosage forms provided herein may further comprise one or more additional active ingredients. Apomorphine, or a pharmaceutically acceptable salt thereof, may be administered as part of a pharmaceutical composition as described herein. Suitable apomorphine compositions, including sublingual films, are described in U.S. Pat. Nos. 8,414,922 and 9,044,475, both of which are incorporated herein by reference.

Compositions of the present disclosure may be formulated for sublingual or buccal administration. In some embodiments, the compositions are formulated for sublingual administration. In some embodiments, the pharmaceutical composition is a sublingual film. In some embodiments, the sublingual film is a bilayer film having a first layer comprising apomorphine or a pharmaceutically acceptable salt thereof and a second layer comprising a pH-modifier. In some embodiments, the pH-modifier is pyridoxine hydrochloride. In some embodiments, the sublingual film further comprises a permeation enhancer. In some embodiments, the bilayer film comprises a first layer comprising from about 10 mg to about 60 mg of apomorphine or a pharmaceutically acceptable salt thereof and a second layer comprising from about 20 to about 70 wt % pyridoxine. In some embodiments, following sublingual administration, the film produces an average circulating apomorphine plasma concentration of at least 3 ng/mL within 20 minutes. In some embodiments, the apomorphine or a pharmaceutically acceptable salt thereof provided in the composition is apomorphine hydrochloride. In some embodiments, the apomorphine or a pharmaceutically acceptable salt thereof provided in the composition is apomorphine hydrochloride hemihydrate.

In some embodiments, disclosed is a pharmaceutical composition comprising apomorphine or a pharmaceutically acceptable salt thereof in admixture with a suitable diluent, carrier, or excipient. Non-limiting examples of the unit dosage forms include a film, lozenge, orally disintegrating tablet, gel, liquid solution, suspension, or powder.

Apomorphine, or a pharmaceutically acceptable salt thereof, may be formulated for sublingual or buccal administration. In some embodiments, apomorphine or a pharmaceutically acceptable salt thereof is formulated for sublingual administration.

To reduce the occurrence of adverse events related to systemic spikes of apomorphine plasma levels, pharmaceutical compositions disclosed herein (e.g., pharmaceutical unit dosage forms) may produce circulating levels that are sufficiently high to be therapeutically effective and are sufficiently low to reduce the occurrence of adverse events. For example, films may produce a Cₘₐₓ in the range of 2.6 ng/mL to 20 ng/mL, 2.6 ng/mL to 15 ng/mL, or 2.6 ng/mL to 10 ng/mL upon administration to oral mucosa (e.g., sublingual mucosa).

In some embodiments, a pharmaceutical unit dosage form described herein contains from 2 mg to 60 mg (e.g., from 8 mg to 45 mg) of apomorphine or a pharmaceutically acceptable salt thereof. Certain exemplary pharmaceutical unit dosage forms may contain 10.0±2.0 mg, 12.5±2.5 mg, 15.0±2.5 mg, 17.5±2.5 mg, 20.0±5.0 mg, 25.0±5.0 mg, 30.0±10.0 mg, 30.0±5.0 mg, 35.0±10.0 mg, or 35.0±5.0 mg of apomorphine or a pharmaceutically acceptable salt thereof. The pharmaceutical unit dosage form may contain from 10% (w/w) to 60% (w/w) of apomorphine or a pharmaceutically acceptable salt thereof (e.g., from 20% (w/w) to 60% (w/w), from 30% (w/w) to 60% (w/w), or from 40% (w/w) to 60% (w/w)) relative to the weight of the pharmaceutical unit dosage form.

The pharmaceutical unit dosage forms described herein can include apomorphine microparticles having a D50 of from 1 pm to 500 pm (e.g., from 1 pm to 100 pm or 1 pm to 50 pm). The starting microparticles can be microspheres can be made of apomorphine or a pharmaceutically acceptable salt thereof and can be predominantly crystalline. Apomorphine or a pharmaceutically acceptable salt thereof can be encapsulated in the microsphere or included in a dissolved drug microsphere. In an alternative approach, the pharmaceutical formulations described herein can include apomorphine particles having an effective particle size of less than about 1 pm (i.e., nanoparticulate formulations). Apomorphine or a pharmaceutically acceptable salt thereof can be encapsulated in the microsphere or included in a dissolved-drug microsphere. These apomorphine particles can be made by using any method known in the art for achieving the desired particle sizes. Useful methods include, for example, milling, homogenization, supercritical fluid fracture, or precipitation techniques. Exemplary methods are described in U.S. Pat. Nos. 4,540,602; 5,145,684; 5,518,187; 5,718,388; 5,862,999; 5,665,331; 5,662,883; 5,560,932; 5,543,133; 5,534,270; and 5,510,118; 5,470,583, each of which is specifically incorporated by reference.

The pharmaceutical compositions described herein can provide a rapid-dissolving, rapid absorption solid dosage form that includes apomorphine or a pharmaceutically acceptable salt thereof. Non-limiting examples of a rapid absorption solid dosage form include, for example, orally disintegrating tablets and orally disintegrating films. In some embodiments, the rapid absorption solid further comprises a pH neutralizing agent. Inclusion of a pH neutralizing agent may facilitate absorption of apomorphine.

A pharmaceutical composition disclosed herein may be provided as a unit dosage form that is a film comprising apomorphine or a pharmaceutically acceptable salt thereof. Apomorphine or a pharmaceutically acceptable salt thereof may be present in the films as a solid solution in a polymeric carrier. Films may furthermore undergo rapid dissolution or rapid disintegration in the oral media (e.g., saliva), e.g., within about 10 minutes (e.g., within about 5 minutes, within about 3 minutes, within about 2 minutes, or within about 1 minute).

Provided herein are formulations comprising apomorphine or a pharmaceutically acceptable salt thereof, and one or more pharmaceutically acceptable excipients. Non-limiting examples of pharmaceutically acceptable excipients includes, any binder, filler, adjuvant, carrier, solubilizer, antioxidant, buffering agent, permeation enhancer, hydrolyzed starches, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, vehicle, disintegrants, or lubricant which has been approved by the United States Food and Drug Administration as being acceptable for use in humans or domestic animals.

The formulations described herein can include an antioxidant system (e.g., a combination of at least two antioxidants) in a unit dosage form described herein. Antioxidants are known in the art. Non-limiting examples of antioxidants that may be included in the dosage form are thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, or penta-, hexa- or heptathionine-sulphoximine), metal chelators (e.g, a-hydroxy-fatty acids, palmitic acid, phytic acid, lactoferrin, citric acid, lactic acid, succinic acid, malic acid, humic acid, bile acid, bile extracts, bilirubin, biliverdin, EDTA, EGTA, or DTPA or a salt thereof), metabisulfite salts (e.g., sodium metabisulfite or potassium metabisulfite), bisulfite salts (e.g., sodium bisulfite or potassium bisulfite), sodium thiosulfate, vitamins and vitamin derivatives (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, or trihydroxybutyrophenone), benzoates (e.g., coniferyl benzoate), cyclodextrins (e.g., P-cyclodextrin or sulfobutyl-P-cyclodextrin), uric acid, mannose, propyl gallate, selenium (e.g., selenium-methionine), stilbenes (e.g., stilbene oxide and trans-stilbene oxide), and combinations thereof.

The formulations described herein can include a permeation enhancer. The permeation enhancer may be, for example, an ionic surfactant, nonionic surfactant, polysorbate, derivatives of tocopherol, poloxamer, monoglyceride, diglyceride, fatty acid, fatty alcohol, or mixtures thereof. In some embodiments, the permeation enhancer is glycerol monostearate.

The formulations described herein can include a pH-modifying agent. In some embodiments, the pH modifying agent is an inorganic base (e.g., aluminum hydroxide, aluminosilicates, calcium hydroxide, magnesium hydroxide, potassium hydroxide, sodium hydroxide, calcium carbonate, iron carbonate, magnesium carbonate, zinc carbonate, sodium carbonate, potassium carbonate, sodium bicarbonate, potassium bicarbonate, sodium phosphate monobasic, sodium phosphate dibasic, sodium phosphate tribasic, potassium phosphate monobasic, potassium phosphate dibasic, potassium phosphate tribasic, mixtures thereof, and any inorganic base described herein). In other embodiments, the pH modifying agent is an organic base (e.g., acetate salts, citrate salts, stearate salts, laurate salts, proprionate salts, lactate salts, succinate salts, oxalate salts, tartrate salts, glycolate salts, galacturonate salts, glucuronate salts, alginate salts, sorbate salts, caprylate salts, carboxymethyl cellulose, polyacrylate, and mixtures thereof and amines, such as pyridoxine, meglumine, lysine, Eudragit E, diethanolamine, glycine, citrate, acetate, histidine, N-methyl glucamine, and tris(hydroxymethyl)aminomethane, mixtures thereof, or any organic base described herein).

In some embodiments, the pharmaceutical composition is a sublingual film comprising one or more of (e.g., two or more, three or more, or each of) pyridoxine, sodium metabisulfite, disodium EDTA, menthol, glyceryl monostearate, glycerin, maltodextrin, sucralose, hydroxyethyl cellulose, hydroxypropyl cellulose, and FD&C Blue #1.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

The following examples are meant to illustrate the invention. They are not meant to limit the invention in any way.

### EXAMPLES

### Example 1: Phase 3 Clinical Study of APL-130277

### Methods

### Patients:

Study patients had a diagnosis of Parkinson's disease consistent with UK Brain Bank criteria and were responsive to levodopa, had 2 or more hours of “OFF” time per day with predictable morning “OFF” periods, and were receiving stable doses of anti-parkinsonian medication.

### Study Design:

The study was a phase 3, multicenter, randomized, placebo-controlled trial that included an open-label titration phase, followed by 12 weeks of double-blind treatment. In the titration phase, patients came to the clinic in the practically defined “OFF” state (not having received anti-parkinsonian medication overnight) and were titrated to the dose of apomorphine sublingual film that provided a FULL “ON” response without serious side effects. Titration started with a 10 mg dose; the dose could then be increased on subsequent days in 5 mg increments up to a maximum of 35 mg. FULL “ON” was defined as a response comparable to that obtained with levodopa.

During the double-blind treatment phase, patients were randomized to apomorphine sublingual film at the dose that provided a FULL “ON” response during titration or to matching placebo. Safety and efficacy evaluations were performed in clinic in the practically defined “OFF” state at weeks 0 (randomization visit), 4, 8, and 12. Evaluations at each visit included Part III (motor examination) of the Movement Disorder Society-Unified Parkinson's Disease Rating Scale (MDS-UPDRS) performed pre-dose and at 15, 30, 45, 60, and 90 minutes post-dose, Part II (motor experiences of daily living) of the MDS-UPDRS, Patient Global Impression of Improvement, Clinical Global Impression of Improvement (CGI-I), and the 39-item Parkinson's Disease Questionnaire (PDQ-39). At each visit, adverse events, vital signs, electrocardiogram, and oropharyngeal cavity examination were performed. During the double-blind phase, patients were also directed to self-administer study drug at home for the treatment of up to 5 “OFF” episodes per day.

### Statistical Analysis:

Efficacy analyses were performed on all randomized patients who received at least one post-randomization dose of study drug (modified intention-to-treat population). The primary endpoint was the mean change from pre-dose to 30 minutes post-dose in the MDS-UPDRS Part III Motor Examination score at the 12-week visit. Multiple sensitivity analyses were conducted to support the primary endpoint. The key secondary endpoint was the percentage of patients with a self-rated FULL “ON” response within 30 minutes at the 12-week visit. Safety analyses were performed on patients who received at least one dose of study medication (safety population).

For the primary endpoint, statistical analysis was performed using a mixed-effect model for repeated measures (MMRM). The model included the observed outcomes at weeks 0, 4, 8, and 12 as response values, with treatment group, visit, and interaction between treatment group and visit as fixed factors, and the change in MDS-UPDRS Part III score between pre-dose and 30 minutes post-dose at baseline (the titration visit at which the randomized dose was employed) as a covariate. The key secondary endpoint was analyzed using a generalized linear mixed model for binomial data. The model included the observed outcomes at weeks 0, 4, 8, and 12 as response values, with treatment group, visit, and interaction between treatment group and visit as fixed factors, and the assessment at baseline as a covariate.

### Results

### Patients:

A total of 214 patients were screened and 141 entered the open-label titration phase and received at least one dose of apomorphine sublingual film. Thirty-two patients discontinued during the titration phase; 8.5% were due to adverse events. The doses of apomorphine sublingual film resulting in a FULL “ON” response at the end of titration were; 10 mg (18.3%), 15 mg (26.6%), 20 mg (21.1%), 25 mg (19.3%), 30 mg (8.3%), and 35 mg (6.4%). One hundred nine patients were randomized to apomorphine sublingual film (n=54) and placebo (n=55). Of those on active treatment or placebo, respectively, 34 (63.0%) and 46 (83.6%) patients completed the study; 15 (27.8%) and 5 (9.1%) discontinued due to adverse events. Baseline characteristics of randomized patients are provided in Table 1 and were similar between groups.

TABLE 1
Baseline Characteristics

| | Apomorphine | |
|---|---|---|
| | sublingual film | Placebo |
| Characteristic | n = 54 | n = 55 |
| Age yr, mean (SD) | 62.9 (9.79) | 62.5 (8.12) |
| Sex, male, n (%) | 37 (68.5) | 31 (56.4) |
| Race, n (%) | | |
| White | 50 (92.6) | 51 (92.7) |
| Other | 4 (7.4) | 4 (7.3) |
| Time since diagnosis, yr, mean (SD) | 8.7 (4.25) | 9.3 (3.84) |
| Time since motor fluctuations started, | 4.7 (3.92) | 4.5 (3.78) |
| yr, mean (SD) | | |
| “ON” state Modified Hoehn and Yahr | | |
| score, n (%) | | |
| 1 or 1.5 | 0 | 1 (1.8) |
| 2 or 2.5 | 49 (90.7) | 42 (76.4) |
| 3 or 4 | 5 (9.3) | 11 (20.0) |
| Missing | 0 | 1 (1.8) |
| MDS-UPDRS part III (pre-dose), | 43.2 (15.17) | 43.1 (14.38) |
| mean (SD)^{a} | | |
| “OFF” episodes per day, Number, | 3.9 (1.17) | 3.8 (1.40) |
| mean (SD) | | |
| Types of “OFF” episodes experienced, | | |
| n (%) | | |
| Morning akinesia | 46 (85.2) | 44 (80.0) |
| Wearing “OFF” | 54 (100) | 54 (98.2) |
| Delayed “ON” | 29 (53.7) | 43 (78.2) |
| Dose failure | 22 (40.7) | 23 (41.8) |
| Sudden “OFF” | 26 (48.1) | 32 (58.2) |
| Total daily levodopa dose, mean | 1059 (563) | 1008 (562) |
| mg (SD) | | |
| Concomitant Parkinson's disease | | |
| medications, n (%) | | |
| Levodopa-containing agents | 54 (100) | 55 (100) |
| Dopamine agonists | 30 (55.6) | 31 (56.4) |
| Monoamine oxidase B inhibitors | 22 (40.7) | 24 (43.6) |
| Amantadine | 8 (14.8) | 16 (29.1) |
| Catechol-O-methyltransferase inhibitors | 5 (9.3) | 5 (9.1) |

^{a}Baseline score refers to the pre-dose MDS-UPDRS Part III motor examination score at the baseline visit (time of randomized apomorphine sublingual film dose).
MDS-UPDRS, Movement Disorder Society-Unified Parkinson's Disease Rating Scale;
SD, standard deviation.

Patients experienced a mean of 3.9 “OFF” episodes per day and were taking a mean of 1,033 mg (range, 400-2,940 mg) of levodopa per day.

### Efficacy:

Results for the primary and secondary endpoints are presented in Table 2.

TABLE 2
Results for Primary and Secondary Endpoints

| | | Apomorphine | | | |
|---|---|---|---|---|---|
| | | sublingual | | | |
| | | film | Placebo | | |
| Hierarchy | Endpoint | n = 54 | n = 55 | Difference | P value |
| 1 | Primary endpoint: LSM | −11.1 points | −3.5 | −7.6 | P = 0.0002 |
| | change (95% CI) and | (−14.0, −8.2) | points | points | |
| | treatment difference in | | (−6.1, | (−11.5, | |
| | MDS-UPDRS Part III score | | −0.9) | −3.7) | |
| | from pre-dose to 30 minutes | | | | |
| | post-dose at week 12 | | | | |
| 2 | Key secondary endpoint: | 35.0 | 16.0 | 2.81 | P = 0.0426 |
| | Self-rated FULL “ON” | (21, 53) | (8, 30) | (1.04, | |
| | response within 30 minutes | | | 7.64) | |
| | at week 12, % response rate, | | | | |
| | estimated OR (95% CI) | | | | |

Secondary endpoints

| | | | | | |
|---|---|---|---|---|---|
| 3 | Self-rated FULL “ON” | 31.0 | 14.0 | 2.80 | P = 0.0501 |
| | response within 30 minutes | (18, 48) | (7, 27) | (1.00, | |
| | post-dose with effect for at | | | 7.84) | |
| | least 30 minutes at | | | | |
| | week 12, % response rate, | | | | |
| | estimated OR (95% CI) | | | | |
| 4 | PGI-I improved at week 12, | 37.0 | 20.0 | — | P = 0.0615 |
| | % | | | | |
| 5 | CGI-I improved at week 12, | 40.7 | 20.0 | — | P = 0.0270 |
| | % | | | | |
| 6 | LSM change (95% CI) and | 0.995 points | 2.095 | −1.100 | P = 0.2906 |
| | treatment difference from | (−0.559, | points | points | |
| | baseline to week 12 in | 2.549) | (0.749, | (−3.159, | |
| | MDS-UPDRS Part II Motor | | 3.440) | 0.959) | |
| | Aspects of Experience of | | | | |
| | Daily Living score - LSM | | | | |
| 7 | FULL “ON” % response | 78.7 | 31.1 | 47.6 | P < 0.0001 |
| | rate (95% CI) at 30 minutes | (64.2, 93.2) | (19.3, | (28.8, | |
| | post-dose based on home | | 43.0) | 66.4) | |
| | dosing diary | | | | |
| 8 | Mean change (95% CI) and | 0.309 points | −1.671 | 1.979 | P = 0.3447 |
| | treatment difference from | (−2.748, | points | points | |
| | baseline to week 12 in | 3.366) | (−4.442, | (−2.162, | |
| | PDQ-39 summary index | | 1.101) | 6.120) | |
| | score | | | | |
| 9 | Mean change (95% CI) and | −6.4 points | −3.0 | −3.4 | P = 0.0390 |
| | treatment difference in | (−8.8, −4.0) | points | points | |
| | MDS-UPDRS Part III score | | (−5.1, | (−6.7, | |
| | from pre-dose to 15 minutes | | −0.8) | −0.2) | |
| | post-dose at week 12 | | | | |
| 10 | Median time (95% CI) to | 21.2 | NE | 3.4 | P < 0.0001 |
| | medication effect at week | (15.0, 27.0) | (42.7, | (1.99, | |
| | 12, min, HR | | NE) | 5.69) | |

CGI-I, Clinician Global Impression of Improvement; CI, confidence interval; HR, hazard ratio; LSM, least squares mean; MDS-UPDRS, Movement Disorder Society-Unified Parkinson's Disease Rating Scale; NE, not evaluable; OR, odds ratio; PDQ-39, Parkinson's Disease Questionnaire; PGI-I, Patient Global Impression of Improvement

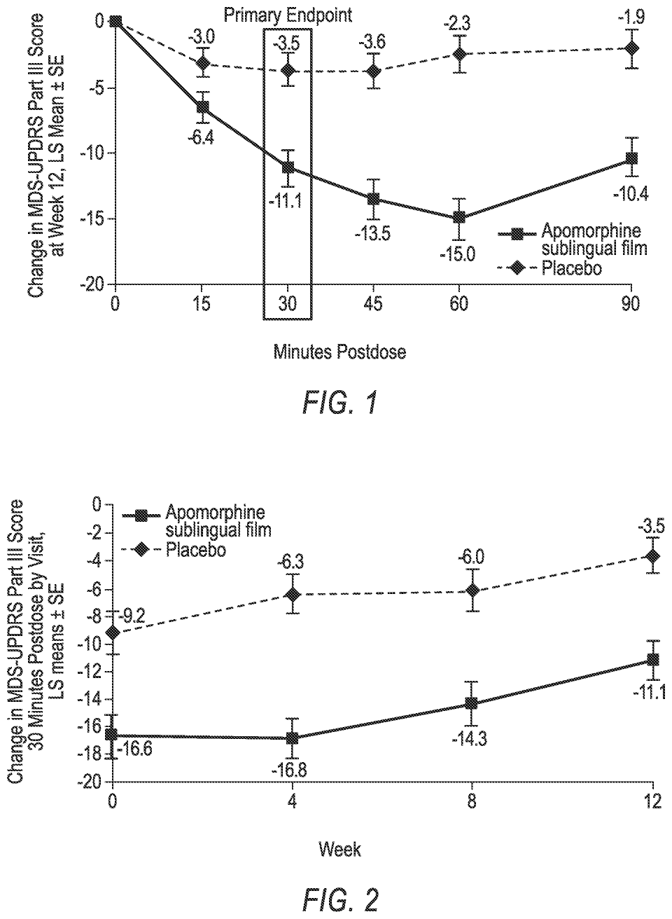

The primary endpoint (change from pre-dose to 30 minutes post-dose at week 12 in MDS-UPDRS Part III) was significantly improved for patients receiving apomorphine sublingual film compared with placebo (−11.1±1.46 vs −3.5±1.29; least squares mean difference −7.6±1.96; P=0.0002). These results were supported by all pre-specified sensitivity analyses. Separation from placebo at week 12 was seen at all measured post-dose time points, from 15 through 90 minutes (FIG. 1); similar benefits were observed at each visit (FIG. 2).

The estimated response rate for the key secondary endpoint (FULL “ON” within 30 minutes at week 12) was also significantly greater for apomorphine sublingual film compared with placebo (35.0% vs 16.0%, estimated odds ratio 2.81; P=0.0426). The percentage of patients at week 12 who reported a FULL “ON” response within 30 minutes with effect lasting at least 30 minutes with active treatment was 31.0% compared with 14.0% with placebo. Home dosing diaries noted that patients treated with apomorphine sublingual film were more likely to report achieving a FULL “ON” within 30 minutes of dosing compared with placebo-treated patients (78.7% vs 31.1%; P<0.0001).

### Safety:

Treatment-emergent adverse events occurring in ≥5% of patients in the open-label titration and double-blind treatment phases are presented in Table 3.

TABLE 3
Treatment-Emergent Adverse Events

| TEAEs > 5% in the open-label | Apomorphine sublingual film | |
|---|---|---|
| titration phase, n (%) | N = 141 | |
| Any | 82 (58.2) | |
| Nausea | 29 (20.6) | |
| Yawning | 17 (12.1) | |
| Dizziness | 16 (11.3) | |
| Somnolence | 16 (11.3) | |
| Headache | 11 (7.8) | |
| Rhinorrhea | 9 (6.4) | |
| Chills | 8 (5.7) | |
| TEAEs > 5% in the double-blind | Apomorphine | Placebo |
| treatment phase, n (%) | film n = 54 | n = 55 |
| Any | 48 (88.9) | 25 (45.5) |
| Nausea | 15 (27.8) | 2 (3.6) |
| Somnolence | 7 (13.0) | 1 (1.8) |
| Dizziness | 5 (9.3) | 0 |
| Fatigue | 4 (7.4) | 0 |
| Rhinorrhea | 4 (7.4) | 0 |
| Vomiting | 4 (7.4) | 0 |
| Dry mouth | 3 (5.6) | 0 |
| Fall | 3 (5.6) | 1 (1.8) |
| Headache | 3 (5.6) | 0 |
| Hyperhidrosis | 3 (5.6) | 2 (3.6) |
| Lacerations | 3 (5.6) | 0 |
| TEAEs related to oropharyngeal | | |
| disorders ≥ 2% in the double-blind | Apomorphine | Placebo |
| treatment phase, n (%) | film n = 54 | n = 55 |
| Oral mucosal erythema | 4 (7.4) | 2 (3.6) |
| Dry mouth | 3 (5.6) | 0 |
| Glossodynia | 2 (3.7) | 0 |
| Lip edema | 2 (3.7) | 0 |
| Lip swelling | 2 (3.7) | 0 |
| Oropharyngeal swelling | 2 (3.7) | 0 |
| Throat irritation | 2 (3.7) | 0 |

TEAE, treatment-emergent adverse event.

Three patients experienced serious adverse events; 2 occurred in patients treated with apomorphine sublingual film (1 patient with known cardiac risk factors had a fatal cardiac arrest, and 1 patient had congestive cardiac failure and hypokalemia, which resolved) and 1 occurred in a placebo-treated patient (encephalopathy and acute kidney injury, which resolved). Nausea and somnolence were reported more frequently with apomorphine sublingual film than placebo but were generally mild and transient. Oropharyngeal treatment-emergent adverse events were reported in 31.5% of patients receiving apomorphine sublingual film, and led to treatment discontinuation in 16.7% of individuals. These spontaneously improved or resolved if treatment was discontinued. Orthostatic hypotension, syncope, dyskinesia, hallucinations, prolongation of the QT interval, and impulse control disorders were rare (incidence ≤2%) or did not occur. There were no clinically meaningful differences between treatment groups in vital signs, electrocardiograms, laboratory parameters, or physical examination. There was no obvious dose relationship with any treatment-emergent adverse events of special interest.

### Discussion

An effective, well-tolerated, easy-to-use, patient-acceptable, on-demand therapy for “OFF” episodes in patients with Parkinson's disease remains an important unmet medical need. This double-blind, placebo-controlled study demonstrated that apomorphine sublingual film can rapidly convert many patients with Parkinson's disease from “OFF” to FULL “ON”. Significant benefit of apomorphine sublingual film versus placebo was seen in the change in the MDS-UPDRS motor score at 30 minutes post-dose at the 12-week visit (P=0.0002; primary endpoint) and in the percent of responders (P=0.0426; key secondary endpoint). All pre-specified sensitivity analyses for the primary endpoint confirmed these findings. Separation from placebo was observed as early as 15 minutes (the first time point measured) and persisted through 90 minutes (the last time point measured). A similar benefit of apomorphine sublingual film was observed at each of the study visits. Benefits of treatment with apomorphine sublingual film were supported by multiple secondary endpoints, including CGI-I and time to onset of clinical benefit. Benefits were also noted with self-administration in the home environment; apomorphine sublingual film provided a FULL “ON” response within 30 minutes in 78.7% of treated episodes compared with 31.1% with placebo. Since apomorphine sublingual film does not require co-administration with carbidopa, this treatment modality may be particularly useful for early-morning “OFF” episodes. Collectively, these findings show that apomorphine sublingual film provides an effective treatment for acute “OFF” episodes for many patients without the need for injection.

Apomorphine sublingual film was safe and well tolerated for most patients. Dopaminergic side effects such as nausea, somnolence, and orthostatic hypotension were mild and transient. Treatment with apomorphine sublingual film was also not associated with clinically significant worsening of dyskinesia, orthostatic hypotension, or impulse control. Oropharyngeal treatment-emergent adverse events were reported in 31.5% of patients receiving apomorphine sublingual film. These were usually mild to moderate and tended to resolve; however, they did lead to discontinuation of treatment in 16.7% of individuals.

### Example 2: Effect of APL-130277 on QTc Interval

### Objective and Endpoint

The primary objective of the study was to evaluate the effect of sublingual apomorphine hydrochloride (APL-130277) compared to placebo on QTc intervals in subjects with Parkinson's disease complicated by motor fluctuations.

The primary endpoint was the time-matched change from baseline in QTc, placebo-adjusted and corrected for HR based on the Fridericia correction (QTcF) method (ΔΔQTcF). Assay sensitivity was evaluated by inclusion of a positive control, moxifloxacin.

### Methods

This was a multi-center, phase 2, randomized, double-blind, placebo-controlled, 3-period crossover, positive control, QT evaluation study of sublingual apomorphine hydrochloride in subjects with Parkinson's disease complicated by motor fluctuations (“OFF” episodes). The study population were subjects with Parkinson's disease who were on stable doses of an L-Dopa formulation and stable treatment with adjunctive Parkinson's disease medication regimens, if clinically warranted, showed a drug withdrawal induced “OFF” episode, and met entry criteria. The study commenced with initial screening visits, followed by an open-label dose titration phase to determine the dose for the randomized crossover assessment phase. Patients were then randomized and began treatment (or control).

Doses of 10 mg to 35 mg were included in the randomized crossover assessment phase to mimic exposure in the target population. Where possible, subjects were titrated to a supratherapeutic dose (up to 60 mg) which was 1 or 2 levels above the initial level producing an “ON” response. Patients were also randomized into control populations: placebo and moxifloxacin. Moxifloxacin 400 mg is a comparator of choice in cardiac safety studies.

The investigational product was a sublingual thin film strip, a rectangular bilayer film containing apomorphine hydrochloride. Doses of 10, 15, 20, 24, 30, and 35 mg were available.

Adverse events were monitored throughout the study at each visit for all subjects. Heart rate (HR), respiratory rate (RR), blood pressure (BP) and body temperature were measured at all study visits just prior to dosing (t=0) and at 15, 45, 60 minutes post dosing after the subject had been in a supine position for 5 minutes. BP was also measured within 3 minutes of standing at all time points. 12-lead ECGs were performed using a continuous Holter monitor and a resting ECG device. The following parameters were reported: Heart rate, PR interval (PR=time between start of P wave and start of QRS complex), QRS interval (QRS=time of QRS complex (Q, R, and S waves), RR interval, QT interval, QTcF interval (Fridericia's correction), and QTcB interval (Bazett's correction). On-screen measurements of RR, PR, QRS and QT interval durations were performed and heart rate, QTcF and QTcB were derived from these measurements.

### Results:

For the primary central tendency analysis, the following 7 time points were evaluated: 15, 30, 45, 60 minutes, and 2, 3, and 4 hours post-dose. For each of the 7 time points, the changes from baseline were compared between treatment and placebo using the ΔΔQTcF method and MMRM analysis.

For the primary assay sensitivity analysis, the following 4 time points were evaluated: 60 minutes and 2, 3, and 4 hours post-dose. For each of the 4 time points, the changes from baseline were compared between moxifloxacin and placebo using the ΔΔQTcF method and MMRM analysis.

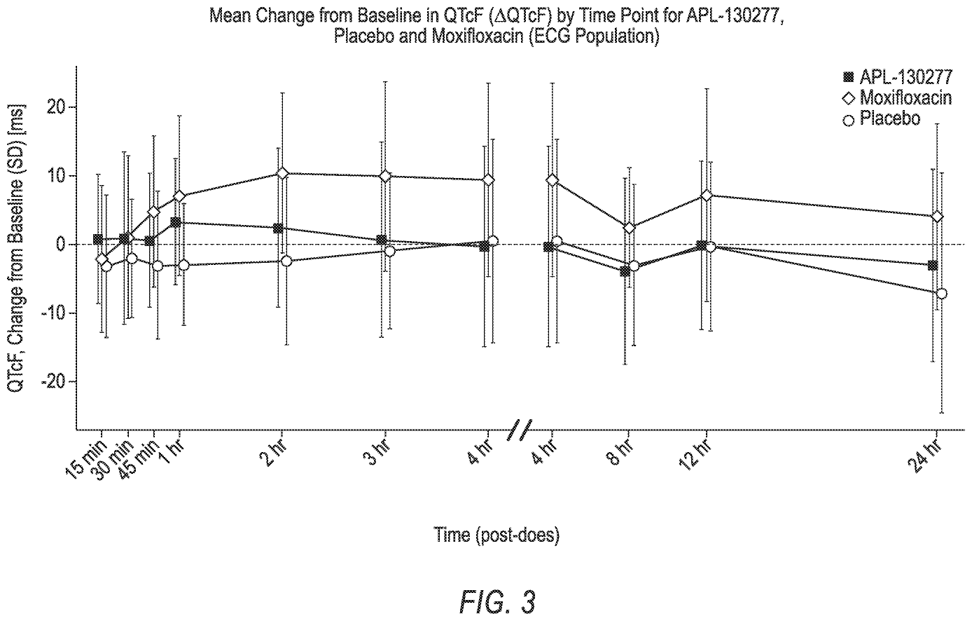

The mean changes from baseline in QTcF (AQTcF) are shown in FIG. 3. As shown in FIG. 3, the mean QTcF changes from baselines for the treatment group increased up to 3.3 ms 1 hour post-dose and declined thereafter. After 4 hours the mean changes from baseline were negative. For placebo, the mean changes were negative at all time points except for 4 hours post-dose (0.5 ms).

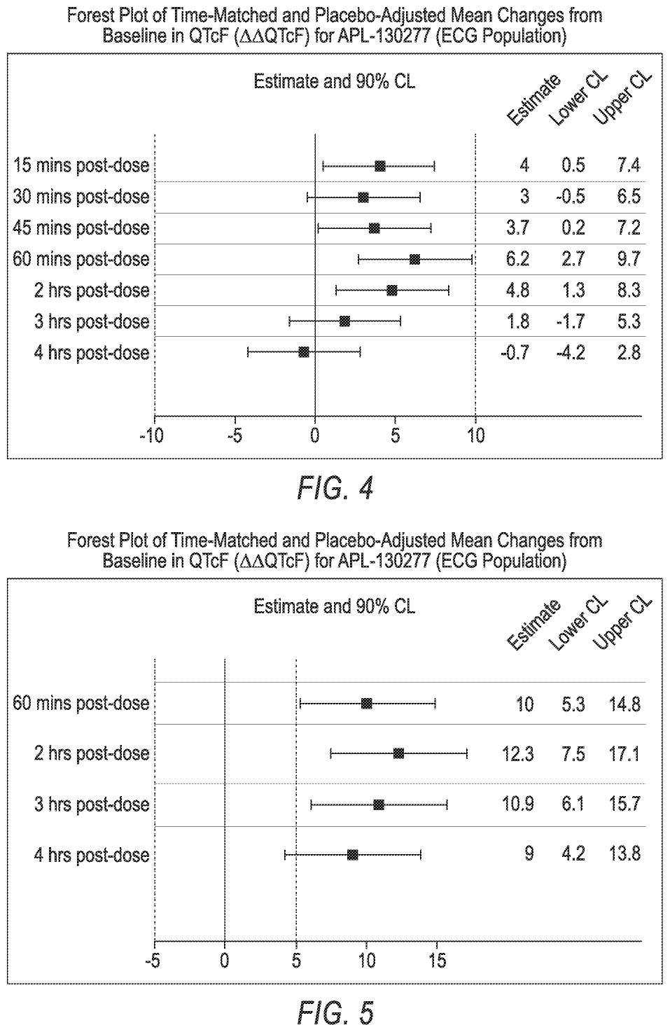

The results for the primary endpoint (ΔΔQTcF) are presented in Table 4 and shown in FIG. 4. The upper limits of the 90% confidence intervals (CIs) for treatment group ΔΔQTcF were below the threshold of 10 ms at all time points, confirming that sublingual apomorphine did not show a significant effect on QTcF.

TABLE 4
Time-Matched and Placebo-Adjusted Mean Changes from Baseline
in QTcF (ΔΔQTcF) for sublingual apomorphine

| | Time-Matched and Placebo-Adjusted Mean | | |
|---|---|---|---|
| | Changes from Baseline in QTcF (ΔΔQTcF) for | | |
| N = 40 | sublingual apomorphine from MMRM analysis | | |
| Time point | Estimate | Lower 90% CL | Upper 90% CL |
| 15 minutes | 4.0 | 0.5 | 7.4 |
| 30 minutes | 3.0 | −0.5 | 6.5 |
| 45 minutes | 3.7 | 0.2 | 7.2 |
| 60 minutes | 6.2 | 2.7 | 9.7 |
| 2 hours | 4.8 | 1.3 | 8.3 |
| 3 hours | 1.8 | −1.7 | 5.3 |
| 4 hours | −0.7 | −4.2 | 2.8 |

CL = confidence limit

The results for changes in baseline in moxifloxacin QTcF (ΔΔQTcF) are summarized in Table 5 and FIG. 5. (APL-130277 is the apomorphine sublingual film)

TABLE 5
Time-Matched and Placebo-Adjusted Mean Changes from Baseline
in QTcF (ΔΔQTcF) for Moxifloxacin

| | Time-Matched and Placebo-Adjusted Mean | | |
|---|---|---|---|
| | Changes from Baseline in QTcF (ΔΔQTcF) for | | |
| N = 40 | moxifloxacin from MMRM analysis | | |
| Time point | Estimate | Lower 90% CL | Upper 90% CL |
| 60 minutes | 10.0 | 5.3 | 14.8 |
| 2 hours | 12.3 | 7.5 | 17.1 |
| 3 hours | 10.9 | 6.1 | 15.7 |
| 4 hours | 9.0 | 4.2 | 13.8 |

CL = confidence limit

As shown in FIG. 3, the mean QTcF changes from baseline (AQTcF) for moxifloxacin were negative at 15 minutes, then increased up to 10.4 ms 2 hours post-dose to a plateau that lasted until 4 hours post-dose. After 4 hours the mean changes declined. The lower limits of the 90% CIs for moxifloxacin were above 5 ms, confirming QTcF prolongation could be detected with the positive control moxifloxacin.

Additional ECG parameters were measured and are presented in Table 6.

TABLE 6
Secondary ECG parameters

| | All timewise 90% CIs excluding zero in time- | | |
|---|---|---|---|
| | matched and placebo-adjusted mean changes | | |
| N = 40 | from baseline for sublingual apomorphine | | |
| ECG | Treatment | Direction of | Time Point |
| parameter | group | Difference | Post-Dose |
| HR (bpm) | Sublingual | Decrease versus | 60 minutes |
| | apomorphine | placebo | |
| | | Increase versus | 3 and 4 hours |
| | | placebo | |
| PR (ms) | Sublingual | Decrease versus | None |
| | apomorphine | placebo | |
| | | Increase versus | 60 minutes |
| | | placebo | |
| QRS (ms) | Sublingual | Decrease versus | None |
| | apomorphine | placebo | |
| | | Increase versus | 2 hours |
| | | placebo | |
| QT (ms) | Sublingual | Decrease versus | 4 hours |
| | apomorphine | placebo | |
| | | Increase versus | 60 minutes |
| | | placebo | |
| QTcB (ms) | Sublingual | Decrease versus | None |
| | apomorphine | placebo | |
| | | Increase versus | 15 minutes, 2 |
| | | placebo | and 3 hours |
| QTcF (ms) | Sublingual | Decrease versus | None |
| | apomorphine | placebo | |
| | | Increase versus | 15, 45 and 60 |
| | | placebo | minutes, 2 hours |

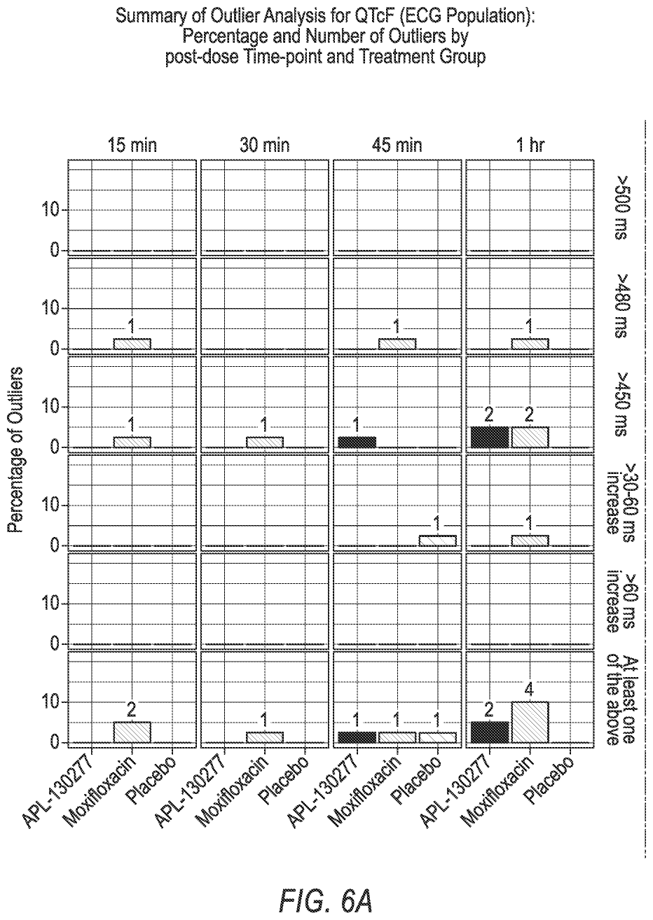

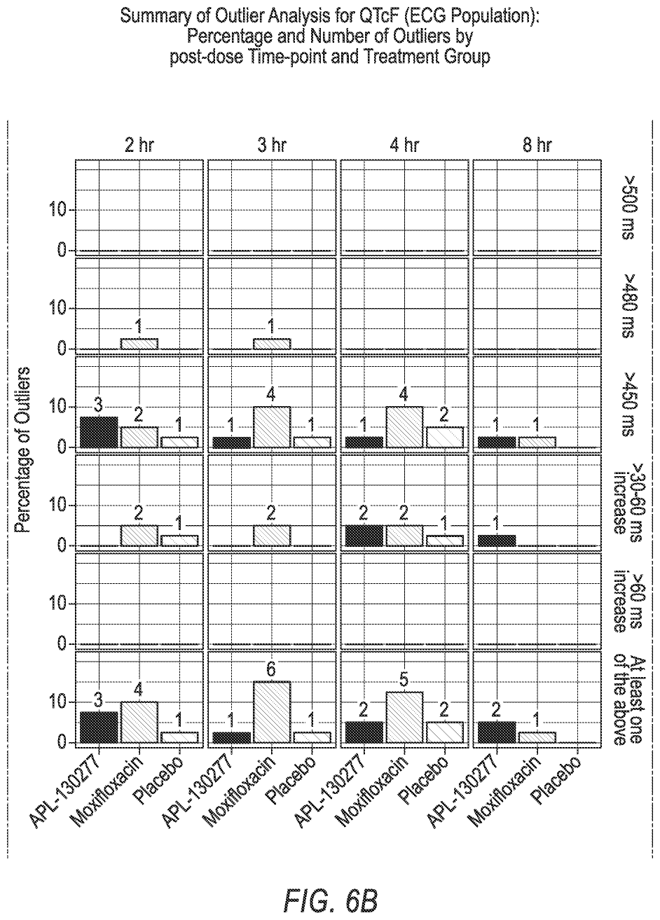

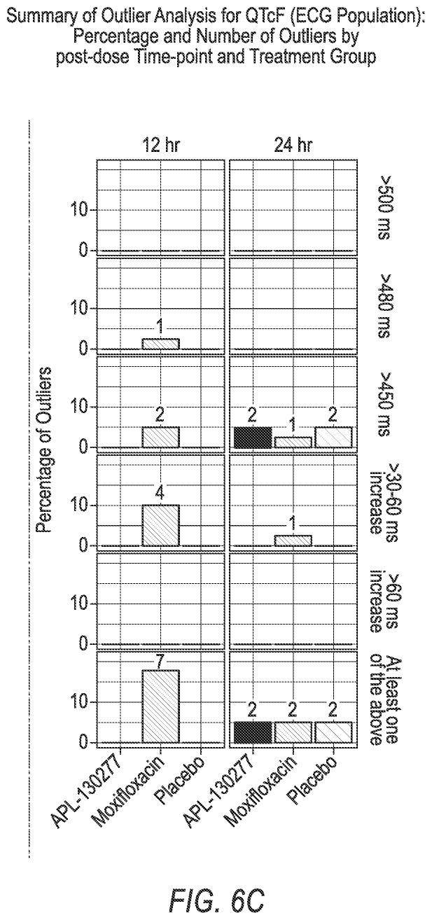

An outlier analysis supplemented the central tendency analysis by determining if there were subjects who had an exaggerated effect on any ECG interval that was not revealed by the central tendency analysis. The results are summarized in FIG. 6. (APL-130277 is the apomorphine sublingual film.)

None of the subjects had a QTcF greater than 500 ms in any of the treatment or control groups. At most one subject in the moxifloxacin group had a QTcF greater than 480 ms per time point. Several subjects per time point had a QTcF greater than 450 ms. The majority of these were with moxifloxacin, and the number of treatment group outliers was similar to placebo. Similar outlier distributions were found for an increase of 30-60 ms in QTcF and no subjects had an increase of greater than 60 ms at any time point.

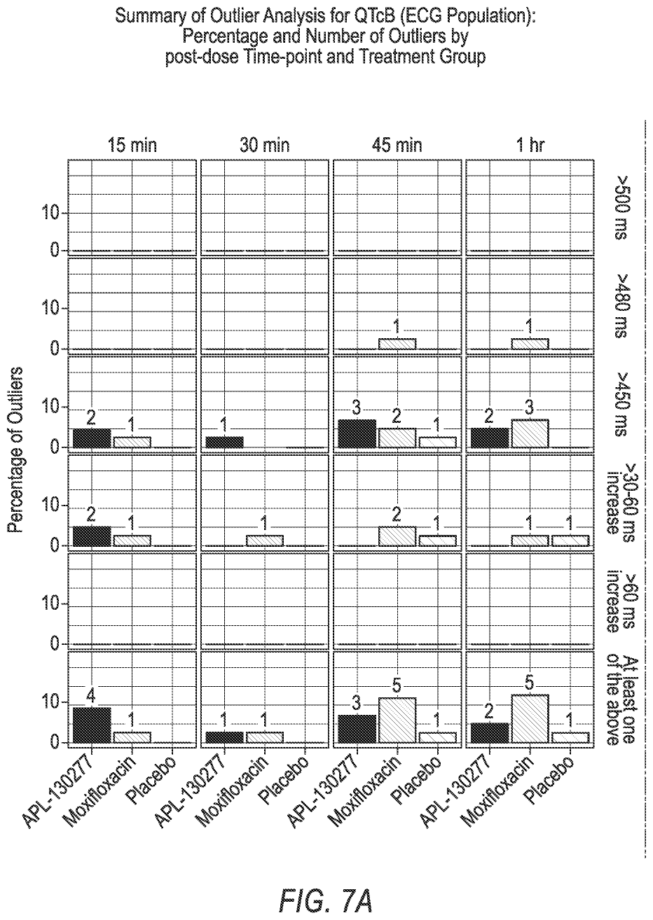

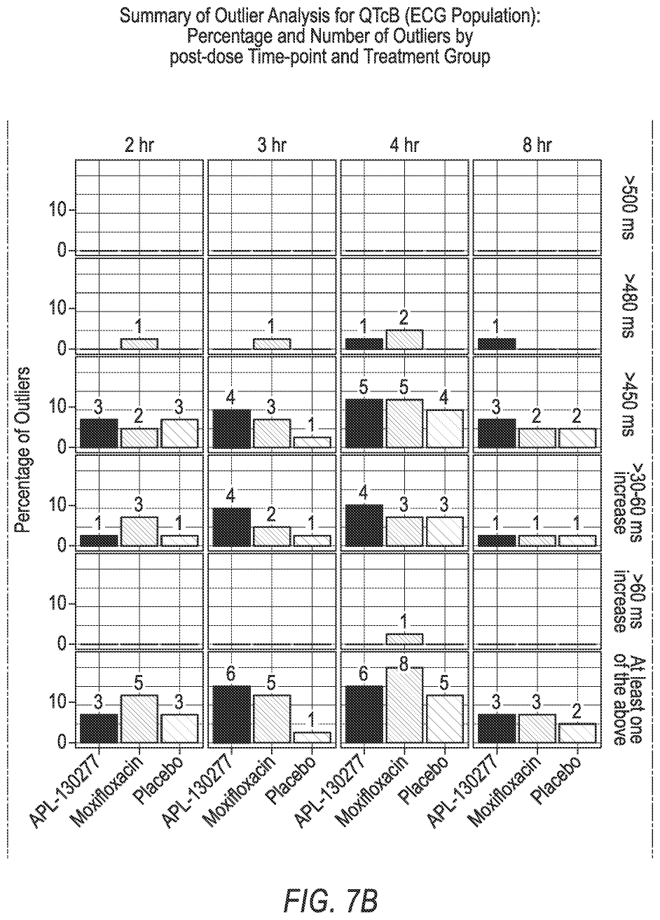

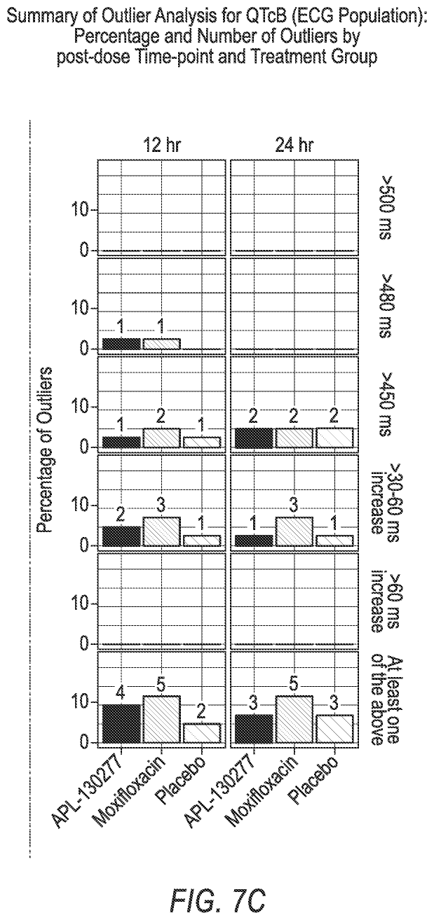

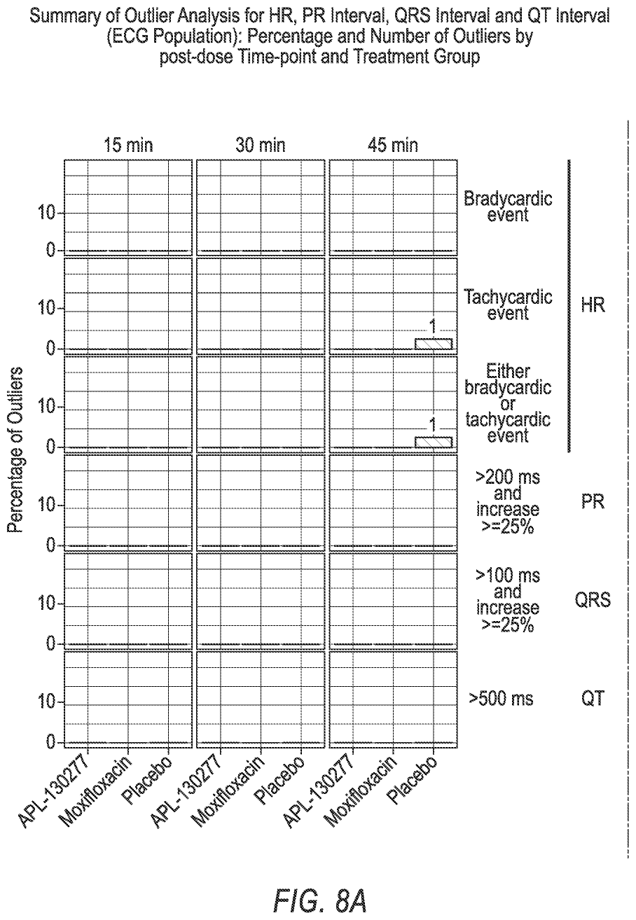

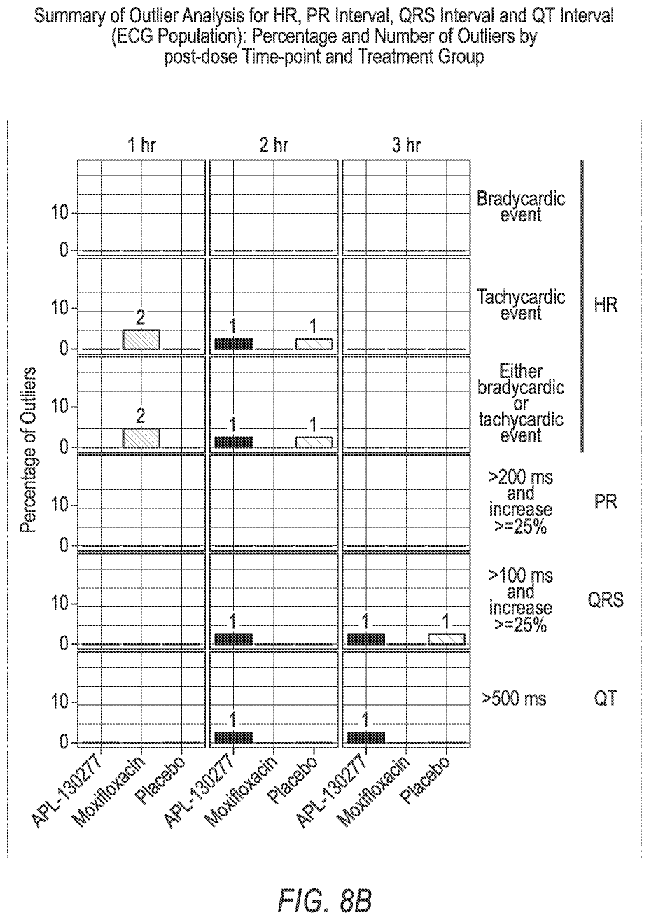

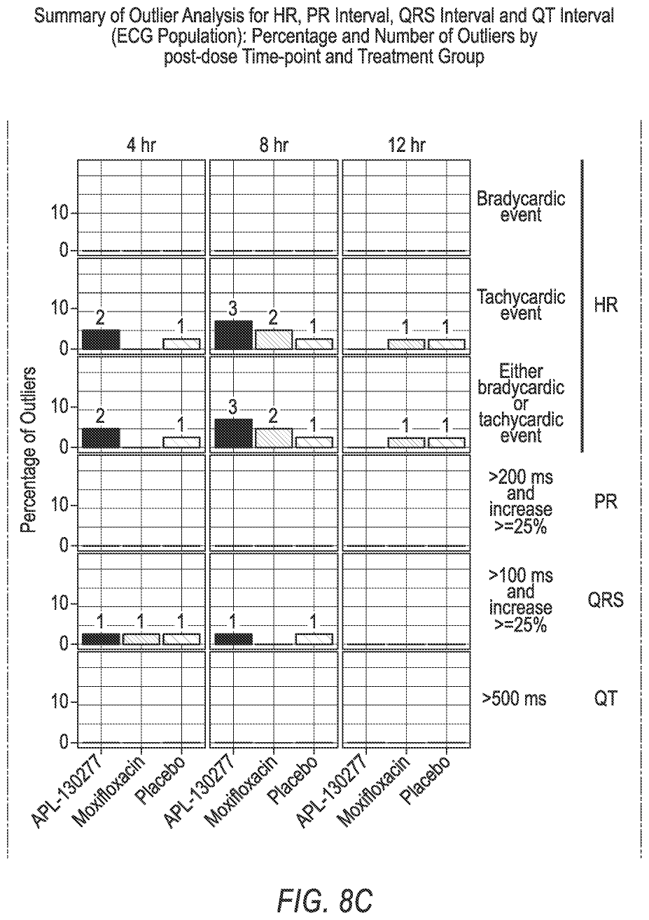

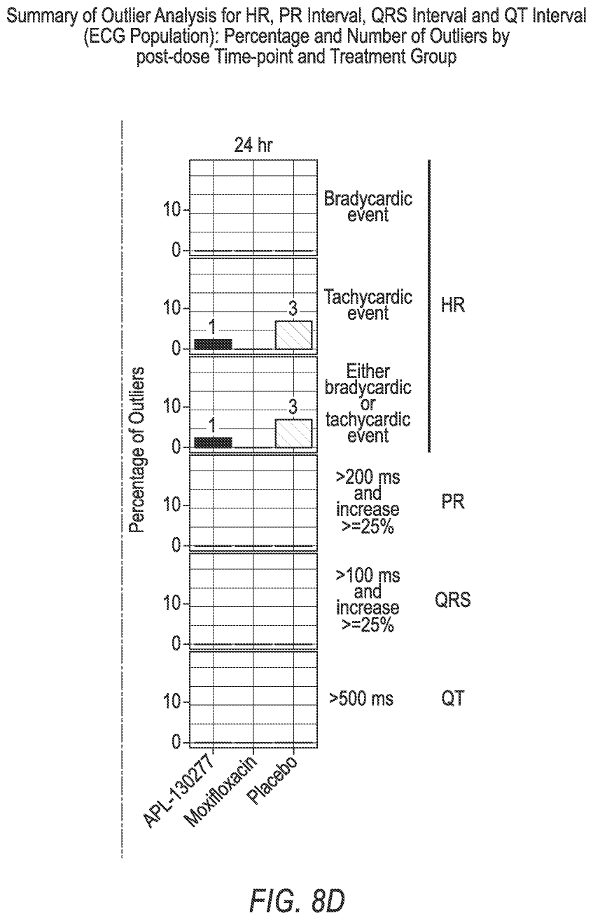

Similar outlier results were observed for QTcB, and other ECG parameters: HR, PR, QRS and QT. See FIGS. 7 and 8. (APL-130277 is the apomorphine sublingual film.)

### Example 3: Comparative Study of APL-130277 Versus Levodopa

Motor responses to apomorphine sublingual film (APL-130277) compared with levodopa in patients with Parkinson's disease and “OFF” Episodes: Post hoc analysis from a Phase 3 study.

Levodopa is the gold standard for the symptomatic treatment of Parkinson's disease (PD); however, motor complications (“OFF” episodes and dyskinesias) occur in up to 90% of patients who have received levodopa for 5-10 years. Apomorphine sublingual film used in this example had the composition shown in Tables 7 and 8.

TABLE 7
Composition of the apomorphine sublingual film layers.

| | Apomorphine | Buffer Layer |
|---|---|---|
| Component | Layer (wt %) | (wt %) |
| Apomorphine Hydrochloride | 53.83 | — |
| Pyridoxine Hydrochloride | — | 67.54 |
| Sodium Hydroxide | — | 11.37 |
| Sodium Metabisulfite | 1.619 | 0.4239 |
| Disodium EDTA, Dihydrate | 1.621 | 0.3696 |
| Emprove ® (-) - Menthol | — | 2.534 |
| Crystals | | |
| Imwitor 491Glyceryl | — | 1.269 |
| Monostearate | | |
| Glycerin, Natural | 4.770 | .1331 |
| Maltodextrin M180 | 9.12 | 0.400 |
| Sucralose | 1.600 | .400 |
| Natrosol 250G Pharm | 7.797 | — |
| Hydroxyethylcellulose | | |
| Natrosol 250L Pharm | 17.203 | 15.50 |
| Hydroxyethylcellulose | | |
| Nisso Hydroxypropylcellulose, | 2.362 | — |
| HPC-SSL | | |
| FD&C Blue #1 Granular, 05603 | 0.07581 | 0.06585 |
| Total | 100.0 | 100.0 |

TABLE 8
Overall composition of the apomorphine sublingual film.

| | | Reference to |
|---|---|---|
| Component | Dry wt % | Standards |
| Apomorphine | 34.50 | USP |
| Hydrochloride^{a} | | |
| Pyridoxine Hydrochloride | 24.20 | USP |
| Sodium Hydroxide | 4.08 | NF |
| Sodium Metabisulfite | 1.19 | NF |
| Disodium EDTA, | 1.17 | USP |
| Dihydrate | | |
| Emprove ® (-) - Menthol | 0.91 | USP |
| Crystals | | |
| Imwitor 491 Glyceryl | 0.46 | USP/NF |
| Monostearate | | |
| Glycerin, Natural | 3.11 | USP |
| Maltodextrin M180 | 5.99 | NF |
| Sucralose | 1.17 | USP/NF |
| Natrosol 250G Pharm | 5.01 | NF |
| Hydroxyethyl Cellulose | | |
| Natrosol 250L Pharm | 16.60 | NF |
| Hydroxyethyl Cellulose | | |
| Nisso Hydroxypropyl | 1.52 | NF/FCC |
| Cellulose, HPC-SSL | | |
| FD&C Blue #1 Granular, | 0.08 | N/A |
| 05603 | | |
| White Ink | Trace | |
| Total | 100 | — |

Apomorphine sublingual film resulted in a significant improvement in Movement Disorder Society Unified Parkinson's Disease Rating Scale Part III Motor Examination (MDS-UPDRS Part III) score at 30 minutes at week 12 (least squares mean difference from placebo, −7.6 points; P=0.0002; primary endpoint).

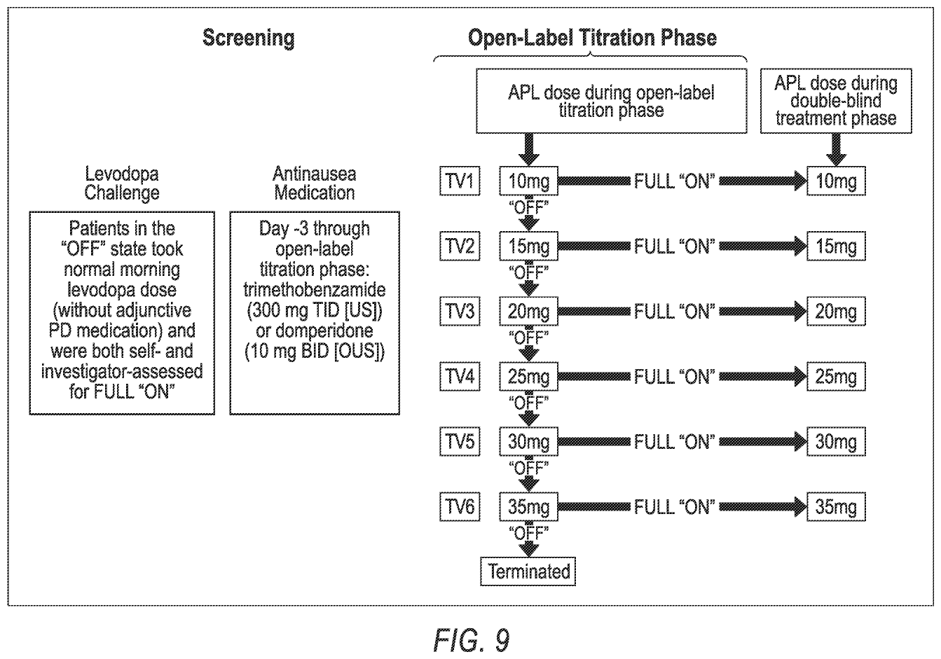

Patients described in Table 9 were enrolled in a study. At screening, patients attended morning in-office visits in the “OFF” state and received their usual morning dose of levodopa without adjunctive PD medications and were both self- and investigator-assessed for a FULL “ON” response (levodopa challenge; FIG. 9). An “OFF” state was accomplished by withholding levodopa and any other adjunctive PD medications after midnight the night before each morning in-office visit. A FULL “ON” response was defined as a response providing benefit regarding mobility, stiffness, and slowness, whereby normal daily activities could be performed and were comparable to or better than normal response to PD medications prior to study enrollment.

TABLE 9
Key eligibility criteria for patients enrolled in the study

| Key Inclusion Criteria | Key Exclusion Criteria |
|---|---|
| Age ≥ 18 years | Atypical secondary |
| Idiopathic PD by UK Brain Bank criteria | parkinsonism |
| Stage I-III by modified Hoehn and Yahr | Major psychiatric disorder |
| scale when “ON” | Mouth cankers/sores |
| Clinically meaningful response to | Previous treatment with: |
| levodopa with well-defined early- | Continuous SC apomorphine |
| morning “OFF” episodes | infusion |
| =1 “OFF” episode per day | Duodopa/duopa |
| Total daily “OFF” time of = 2 hours | SC apomorphine within 7 |
| Receiving stable doses of levodopa QID | days of screening |
| or carbidopa/levodopa extended-release | Currently taking: |
| capsules TID | 5-HT₃ antagonists |
| PD medications stable for ≥ 4 weeks | Selective dopamine |
| MAO-B inhibitors stable for ≥ 8 weeks | antagonists (excluding |
| | quetiapine or clozapine) |
| | Dopamine-depleting agents |

5-HT₃, 5-hydroxytryptamine type 3; MAO-B, monoamine oxidase B; PD, Parkinson's disease; QID, 4 times a day; SC, subcutaneous; TID, 3 times a day.

Following screening, during open-label titration, patients attended sequential morning in-office visits in the “OFF” state and received increasing doses of apomorphine sublingual film from 10-35 mg in 5-mg increments on subsequent days until a FULL “ON” response was achieved within 45 minutes without intolerable side effects (FIG. 9). Motor responses were evaluated using MDS-UPDRS III score, performed predose and at 15, 30, 45, 60, and 90 minutes postdose during screening with levodopa and during the titration visit with the dose of apomorphine sublingual film that resulted in a FULL “ON” response. Motor responses following open-label apomorphine sublingual film and levodopa administration were assessed descriptively in this post hoc analysis

All patients who were successfully titrated and therefore randomized and received ≥1 post randomization dose of study medication (randomized population) were included in the analysis.

Among the 141 patients who entered the open-label titration phase 109 patients (randomized population) were evaluated in this post hoc analysis (levodopa data were missing for 1 patient; Table 10).

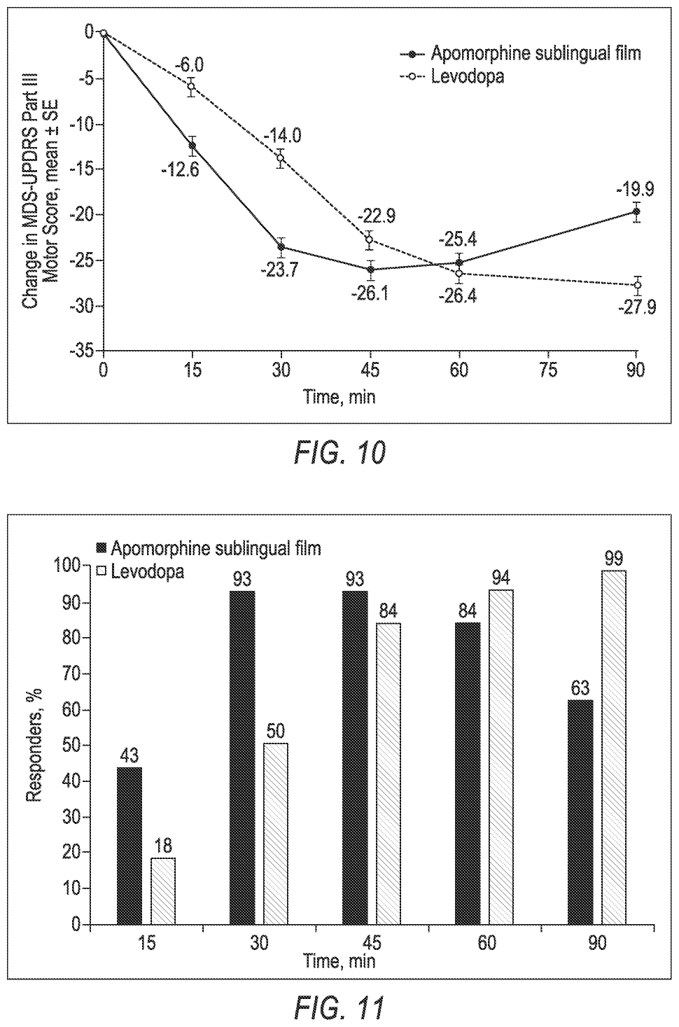

Predose MDS-UPDRS III scores (mean±standard error (SE)) were comparable at screening and titration (43.5±1.34 vs 43.1±1.41). At 15 minutes postdose, the magnitude of motor response (mean±SE) with apomorphine sublingual film was ˜2-fold higher compared with levodopa (−12.6±0.97 versus −6.0±0.72; FIG. 10). Across the time points measured, mean peak response to apomorphine sublingual film occurred earlier compared with the trend observed for levodopa (45 versus 95 minutes). Notably, apomorphine sublingual film was still associated with a clinically meaningful improvement in MDS-UPDRS III score (mean±SE) at 90 minutes postdose (−19.9±1.55). The magnitude of observed peak responses (mean±SE) across the time points measured was comparable (−26.1±1.19 vs −27.9±1.20 for apomorphine sublingual film and levodopa, respectively).

TABLE 10
Demographics and baseline disease characteristics

| | Randomized |
|---|---|
| | Population |
| Characteristic | (n = 109) |
| Age, y, mean (SD) | 62.7 (8.95) |
| Male, n (%) | 68 (62) |
| White, n (%) | 101 (93) |
| Time since PD diagnosis, y, mean (SD) | 9.0 (4.04) |
| Time since motor fluctuations started, | 4.6 (3.83) |
| y, mean (SD) | |
| Modified Hoehn and Yahr score | |
| when “ON”, n (%) | |
| 1 or 1.5 | 1 (1) |
| 2 or 2.5 | 91 (83) |
| 3 | 16 (15) |
| Missing | 1 (1) |
| MDS-UPDRS Part III score (predose), | 43.5 (1a3.97) |
| mean (SD)^{a} | |
| “OFF” episodes per day, n, mean (SD) | 3.9 (1.29) |
| Total daily levodopa dose, mg, mean (SD) | 1033 (561) |

^{a}Baseline score refers to the predose MDS-UPDRS Part III score in the “OFF” state prior to levodopa administration at screening.
MDS-UPDRS, Movement Disorder Society Unified Parkinson's Disease Rating Scale; PD, Parkinson's disease; SD, standard deviation.

In responder analyses, there were more responders (defined as patients with a ≥30% decrease in MDS-UPDRS III score from predose) with apomorphine sublingual film than with levodopa at earlier time points (FIG. 11). Responder rates were 43% vs 18% at 15 minutes postdose and 93% vs 50% at 30 minutes postdose for apomorphine sublingual film vs levodopa, respectively. At 90 minutes postdose, 63% of patients receiving apomorphine sublingual film were still considered responders.

This study has analyzed the comparative efficacy of apomorphine versus levodopa in the treatment of “OFF” episodes in a large number of patients with PD. Doses of apomorphine sublingual film necessary to achieve a FULL “ON” response were associated with a faster onset of motor improvement compared with levodopa, with a similar magnitude of response seen for both agents. Apomorphine sublingual film was also associated with more responders at earlier time points compared with levodopa. Apomorphine sublingual film continued to provide a clinically meaningful improvement in MDS-UPDRS III score at 90 minutes postdose, suggesting that it may offer a durable on-demand treatment for “OFF” episodes.

### Example 4: Efficacy, Safety, and Tolerability of Supratherapeutic Doses of APL-130277 for the Treatment of “OFF” Episodes in Patients with Parkinson's Disease

Levodopa-treated patients with Parkinson's disease (PD) frequently develop motor fluctuations (“OFF” episodes), a common debilitating complication that may negatively impact quality of life. Apomorphine sublingual film has the composition shown in Table 11.

TABLE 11
Composition of the apomorphine sublingual film layers.

| | Apomorphine | Buffer Layer |
|---|---|---|
| Component | Layer (wt %) | (wt %) |
| Apomorphine Hydrochloride | 53.83 | — |
| Pyridoxine Hydrochloride | — | 67.54 |
| Sodium Hydroxide | — | 11.37 |
| Sodium Metabisulfite | 1.619 | 0.4239 |
| Disodium EDTA, Dihydrate | 1.621 | 0.3696 |
| Emprove ® (-) - Menthol | — | 2.534 |
| Crystals | | |
| Imwitor 491Glyceryl | — | 1.269 |
| Monostearate | | |
| Glycerin, Natural | 4.770 | .1331 |
| Maltodextrin M180 | 9.12 | 0.400 |
| Sucralose | 1.600 | .400 |
| Natrosol 250G Pharm | 7.797 | — |
| Hydroxyethylcellulose | | |
| Natrosol 250L Pharm | 17.203 | 15.50 |
| Hydroxyethylcellulose | | |
| Nisso Hydroxypropylcellulose, | 2.362 | — |
| HPC-SSL | | |
| FD&C Blue #1 Granular, 05603 | 0.07581 | 0.06585 |
| Total | 100.0 | 100.0 |

TABLE 12
Overall composition of the apomorphine sublingual film.

| | | Reference to |
|---|---|---|
| Component | Dry wt % | Standards |
| Apomorphine | 34.50 | USP |
| Hydrochloridea | | |
| Pyridoxine Hydrochloride | 24.20 | USP |
| Sodium Hydroxide | 4.08 | NF |
| Sodium Metabisulfite | 1.19 | NF |
| Disodium EDTA, | 1.17 | USP |
| Dihydrate | | |
| Emprove ® (-) - Menthol | 0.91 | USP |
| Crystals | | |
| Imwitor 491 Glyceryl | 0.46 | USP/NF |
| Monostearate | | |
| Glycerin, Natural | 3.11 | USP |
| Maltodextrin M180 | 5.99 | NF |
| Sucralose | 1.17 | USP/NF |
| Natrosol 250G Pharm | 5.01 | NF |
| Hydroxyethyl Cellulose | | |
| Natrosol 250L Pharm | 16.60 | NF |
| Hydroxyethyl Cellulose | | |
| Nisso Hydroxypropyl | 1.52 | NF/FCC |
| Cellulose, HPC-SSL | | |
| FD&C Blue #1 Granular, | 0.08 | N/A |
| 05603 | | |
| White Ink | Trace | |
| Total | 100 | — |

In a Phase 3, 12-week, randomized, double-blind, placebo-controlled study, apomorphine sublingual film was shown to be effective as a treatment for acute “OFF” episodes in patients with PD and was generally well tolerated. Apomorphine sublingual film is typically titrated to the dose needed to achieve a FULL “ON” response. In the present example, the potential benefit of supratherapeutic doses of apomorphine sublingual film was assessed to determine whether higher doses of apomorphine sublingual film than that shown to be initially efficacious can achieve a better “ON” response in patients with PD.

### Study Design

Patients with PD who demonstrated a clinically meaningful response to levodopa and who had an “OFF” episode in response to drug withdrawal were eligible to participate in a Phase 2 QT study (ClinicalTrials.gov: NCT03187301; Table 13). Antiemetics (such as trimethobenzamide or domperidone) were not allowed.

TABLE 13
Patients enrolled in the study

| Key Inclusion Criteria | Key Exclusion Criteria |
|---|---|
| Age ≥ 18 years | Atypical or secondary |
| Idiopathic PD by UK Brain Bank criteria | parkinsonism |
| Stage I-III by modified Hoehn & Yahr | Nausea with use of dopamine |
| scale when “ON” | agonists requiring antiemetics |
| Clinically meaningful response to | Major psychiatric disorders |
| levodopa with or without well-defined | Abnormalities with ECG at |
| “OFF” episodes | |
| Ability to have a drug withdrawal- | screening^{a}: |
| induced “OFF” episode | QTcF ≥ 450 ms (male) |
| Receiving stable doses of: | or ≥ 470 ms (female) |
| Levodopa/carbidopa TID for ≥ 4 weeks | HR < 45 bpm or > 100 bpm |
| Adjunctive PD medications for | QRS duration > 120 ms |
| ≥4 weeks | PR interval > 200 ms |
| MAO-B inhibitors for ≥ 8 weeks | |

^{a}Included arrhythmias, clinically meaningful interval irregularities, structural heart abnormalities, myocardial infarction, presence or history of a pacemaker, or any ECG abnormality that would have interfered with the ability to measure the QT interval or correct the QT interval for HR.
bpm, beats per minute; ECG, electrocardiogram, HR, heart rate, MAO-B, monoamine oxidase B; PD, Parkinson's disease; QTcF, QT interval corrected with Fridericia's method; TID, 3 times a day.

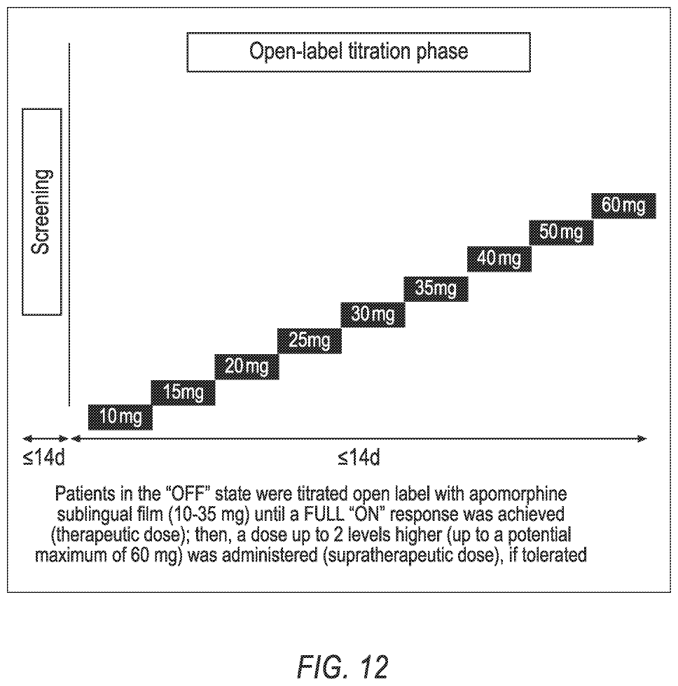

In an open-label fashion, patients in the “OFF” state were titrated at an in-office visit with apomorphine sublingual film (10-35 mg) until a FULL “ON” response was achieved (therapeutic dose); then, a dose up to 2 levels higher (potential maximum of 60 mg) was administered (supratherapeutic dose), if tolerated (FIG. 12). Titration visits were scheduled no more than 2 days apart. During titration visits 3 through 6, patients could receive the next-highest dose of apomorphine sublingual film within 4 hours if another “OFF” episode occurred that day.

### Assessments and Statistical Analyses

Prespecified exploratory efficacy analyses were conducted between the highest dose (supratherapeutic) and the initial therapeutic dose that provided a FULL “ON” response during titration by comparing: (1) changes in Movement Disorder Society Unified Parkinson's Disease Rating Scale (MDS-UPDRS) Part III Motor Examination scores from predose to 30, 60, and 90 minutes postdose using a Mixed Model for Repeated Measures (MMRM) approach; and (2) time to and duration of a FULL “ON” response using Kaplan-Meier analysis. Patients were excluded from efficacy analyses if the therapeutic and supratherapeutic doses were the same. Treatment-emergent adverse events (TEAEs) were collected throughout the study and coded using Medical Dictionary for Regulatory Activities (MedDRA) version 19.1.

### Patients

Forty-eight patients were enrolled, received ≥1 dose of apomorphine sublingual film during titration, and comprised the safety population (Table 14 and Table 15). Thirty-five patients had efficacy assessments during titration at the therapeutic and supratherapeutic doses, and comprised the efficacy population. Among the 13 patients in the safety population excluded from efficacy assessments (Table 14); seven discontinued the study due to TEAEs (n=5; 10%), withdrawal of consent (n=1; 2%), and eligibility criteria not met (intracranial aneurism identified after enrollment [n=1; 2%]); and six were excluded from efficacy analyses because their therapeutic and supratherapeutic doses were identical.

TABLE 14
Patient disposition

| | Total, N |
|---|---|
| Disposition | (%) |
| Screened | 73 (100) |
| Enrolled | 48 (66) |
| Received ≥ 1 dose of apomorphine sublingual | 48 (100) |
| film during titration (safety population) | |
| Assessed for efficacy (efficacy population) | 35 (73) |

TABLE 15
Patient demographics and baseline characteristics

| | Safety Population |
|---|---|
| Characteristic | (N = 48) |
| Age, y, mean (SD) | 64.9 (8.56) |
| Male, % | 63 |
| White, % | 92 |
| Time since PD diagnosis, y, mean (SD) | 8.5 (4.36) |
| Time since motor fluctuations started,^{a} y, mean (SD) | 5.1 (4.03) |
| Total daily levodopa dose, mg, mean (SD) | 632.7 (281.54) |

^{a}n = 47; information was missing for 1 patient. PD, Parkinson's disease; SD, standard deviation.

### Exposure

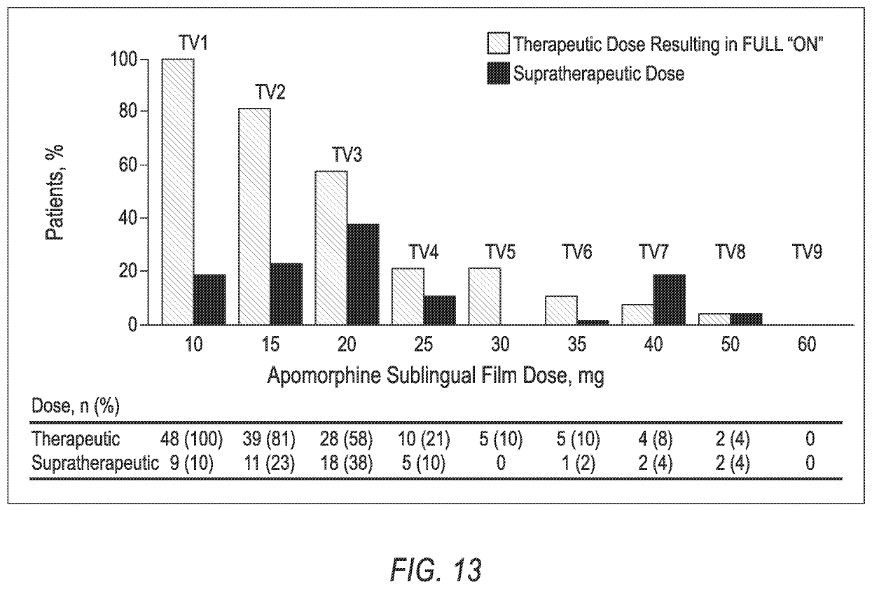

For the majority of patients (79%), the therapeutic dose of apomorphine sublingual film received during titration was ≤20 mg (FIG. 13). Sixteen patients received a supratherapeutic dose that was 1 level higher than the therapeutic dose and 19 patients received a dose 2 levels higher. No patient was titrated to a 60-mg dose.

### Efficacy

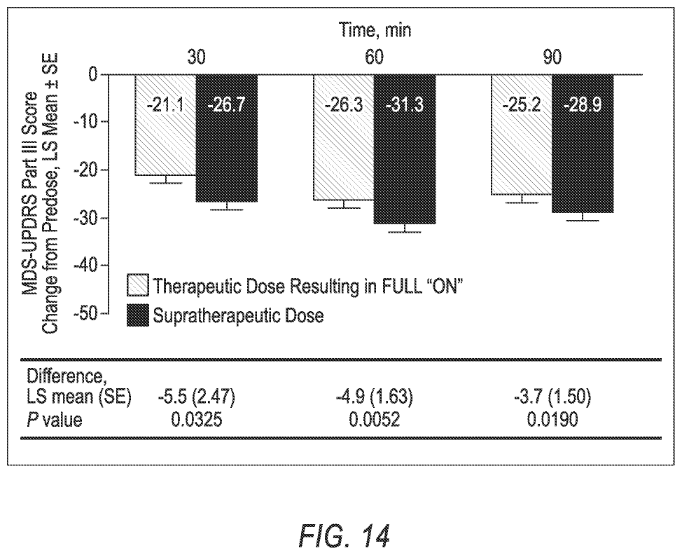

Pre-dose MDS-UPDRS Part III scores in the “OFF” state were similar before therapeutic and supratherapeutic doses of apomorphine sublingual film were administered (mean±standard deviation: 54.3±17.0 and 54.0±15.6). Postdose mean reductions in MDS-UPDRS PART III scores were greater with supratherapeutic doses compared with therapeutic doses at 30, 60, and 90 minutes (FIG. 14). Indeed, improvement was seen at each time point; the largest difference favoring the supratherapeutic dose was observed at 30 minutes postdose (least squares mean±standard error of the difference: −5.5±2.5; P=0.0325) with a sustained improvement observed through 90 minutes postdose (P=0.0190). Additionally, the response seen with supratherapeutic doses at 30 minutes was comparable to that of therapeutic doses at 60 minutes.

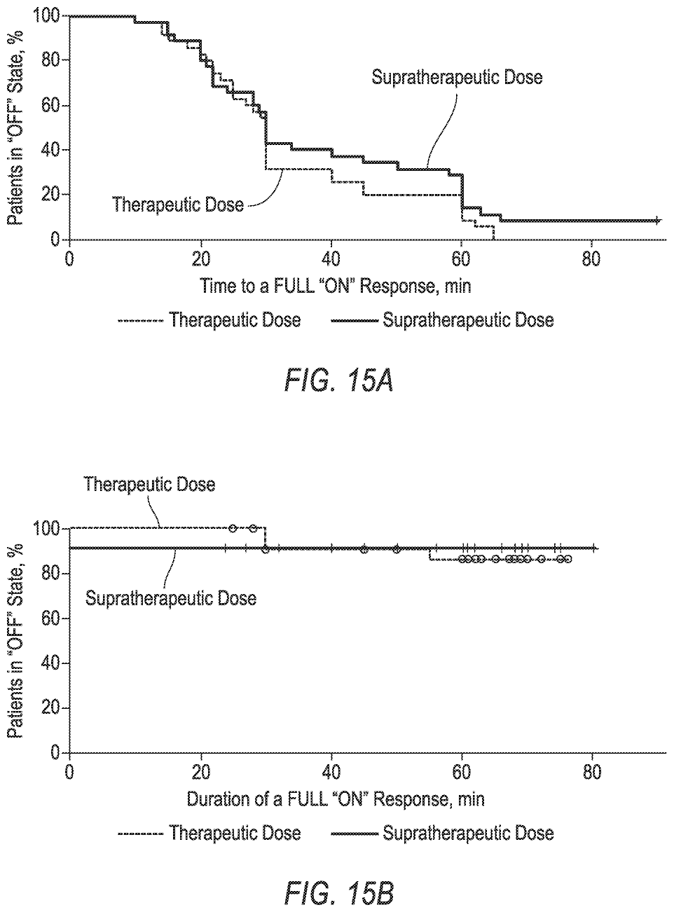

The median time to a FULL “ON” response was 30 minutes and the earliest time to a FULL “ON” response was 10 minutes for both therapeutic and supratherapeutic doses (FIG. 15A). Three patients did not achieve a FULL “ON” response with supratherapeutic doses. The duration of a FULL “ON” response was similar for both therapeutic and supratherapeutic doses with approximately 90% of patients maintaining a FULL “ON” response for=50 minutes (FIG. 15B).

### Safety

Forty-one patients (85%) reported >1 TEAE; the most common was nausea (56%; Table 16). The majority of TEAEs were mild to moderate in intensity (96%) and occurred shortly after dosing, were transient, and resolved without concomitant treatment. Five severe TEAEs were reported in 5 patients during titration; all were considered related to apomorphine sublingual film, led to study discontinuation, and resolved. Nausea occurred in 3 patients; 2 patients received 10 mg at titration visit (TV) 1 and 1 patient received 20 mg at TV3 (concurrent moderate vomiting led to discontinuation in this patient). Somnolence occurred in 1 patient after receiving 10 mg at TV1. Orthostatic hypotension occurred in 1 patient after receiving 50 mg at TV8. No serious TEAEs or deaths were reported during the study.

TABLE 16
TEAEs reported by ≥ 2 patients receiving apomorphine sublingual
film during titration

| | Safety Population, | |
|---|---|---|
| | (N = 48) | |
| Preferred Term | % | Events, n |
| Any TEAE | 85 | 117 |
| Nausea | 56 | 35 |
| Somnolence | 25 | 20 |
| Vomiting | 19 | 9 |
| Dizziness | 17 | 8 |
| Hyperhidrosis | 15 | 7 |
| Hypotension | 8 | 5 |
| Headache | 6 | 3 |
| Orthostatic hypotension | 6 | 6 |
| Yawning | 4 | 3 |
| Blood pressure systolic decreased | 4 | 2 |
| Dyskinesia^{a} | 4 | 2 |
| Vertigo | 4 | 2 |

^{a}n = 47; information was missing for 1 patient. PD, Parkinson's disease; SD, standard deviation.

Despite patient and investigator perception that an initial dose of apomorphine sublingual film achieved a FULL “ON” response, administration of a higher dose further improved motor function. Improvement in MDS-UPDRS PART III scores was seen at each measured time point; the largest increase after a supratherapeutic dose was seen at 30 minutes, with improvement observed up to 90 minutes postdose (last timepoint examined). With a supratherapeutic dose, an equivalent “ON” response was achieved earlier compared with a therapeutic dose. As seen in a previous Phase 3 study, the most frequently reported safety events in this study were those associated with dopamine agonists and no new or unexpected safety findings were identified.

### Example 5: Exposure-Response Model for APL-130277 in PD Patients

A model was developed utilizing efficacy response data from two Phase 2 studies and two Phase 3 studies of apomorphine sublingual film (APL-130277). In all studies, the response data were collected up to 1.5 hours after dosing. Exposure data (i.e., predicted plasma concentrations) were derived from a developed population pharmacokinetic (PK) model based on nine clinical studies.

A longitudinal E-R model was developed relating MDS-UPDRS scores to apomorphine exposure in patients with PD following sublingual administration of APL-130277. The analysis sequence included development of base, full and final models. Apomorphine PK concentrations were generated using empirical Bayes estimates from an independently developed population PK model.

The placebo base E-R model used an asymptotic exponential function to describe the placebo effect on MDS-UPDRS part III scores (motor). The base model for active treatment used a sigmoidal Emax function to quantify the proportional reduction of the baseline PHD measure due to drug effect with the addition of placebo effect.

The model was updated with addition of new data from a later clinical study. An updated population PK model was developed and used to generate individual apomorphine concentration estimates prior to updating the final E-R model. The current analysis initially assessed the predictive performance of the developed base E-R model (without covariates) using new data from subjects not included in the original analysis via an external posterior predictive check (PPC). Using the updated final model, simulations were performed to estimate the apomorphine concentration associated with efficacy (ON state) and duration of effect.

### Results

The previously developed E-R model was updated with new data. The numbers of subjects and MDS-UPDRS assessments in the previous and updated E-R analysis datasets are summarized in Table 17. After adding new subjects and data, the updated dataset consisted of 13192 MDS-UPDRS measurements from nonunique 631 subjects (i.e., subjects enrolling in multiple studies were counted in each study).

TABLE 17
Number of Subjects and MDS-UPDRS Measurements
in the Exposure-Response Analysis Dataset by Study

| | | | | | | | MDS-UPDRS | | | |
|---|---|---|---|---|---|---|---|---|---|---|
| | Subjects and | | Additional Subjects and | | Additional | | Data in | | Subjects and | |
| | MDS-UPDRS | | MDS-UPDRS | | MDS-UPDRS | | Previous Dataset | | MDS-UPDRS | |
| | Data in | | Data in | | Data from | | Missing from | | Data in | |
| | Previous Dataset | | Updated Dataset | | Previous Subjects | | Updated Data | | Updated Dataset | |
| Study | N | PHD | N | PHD | N | PHD | | | N | PHD |
| CTH-105 | 19 | 464 | — | — | — | — | — | — | 19 | 464 |
| CTH-201 | 48 | 558 | — | — | — | — | — | — | 48 | 558 |
| CTH-300 | 141 | 4392 | — | — | — | — | — | — | 141 | 4392 |
| CTH-301 | 257 | 4588 | 168 | 2782 | 38 | 416 | 3 | (8) | 425 | 7778 |
| Total | 465 | 10002 | 168 | 2782 | — | 416 | — | (8) | 633 | 13192 |

| | | |
|---|---|---|
| Total included in the analysis | 631 | 13171 |

PK = pharmacokinetic;
PHD = pharmacodynamic measurement (MDS-UPDRS score);
MDS-UPDRS = Movement Disorder Society′s Unified Parkinson′s Disease Rating Scale

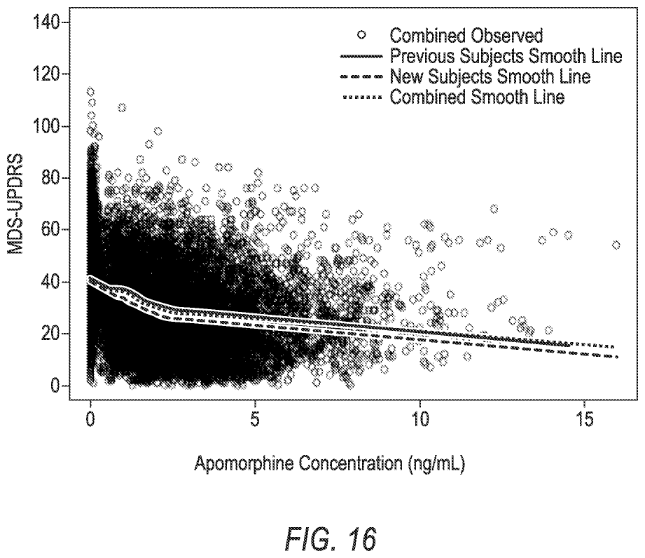

Observed MDS-UPDRS scores versus PK model-predicted apomorphine concentration (at the time of the MDS-UPDRS measurement) for all APL-130277 treatments in all studies are compared between the previous and updated datasets in FIG. 16. The observed MDS-UPDRS Part III Scores decrease with higher predicted apomorphine plasma concentrations. A similar trend was observed for the subjects in the previous analysis and the new subjects in the updated data.

The previous E-R model adequately predicted MDS-UPDRS scores for the new subjects. Therefore, the previous data and new data were pooled and model parameters were re-estimated using the base and final models with the updated dataset.

The E-R model structure in the updated analysis was unchanged from the previous analysis, and consisted of a placebo component modeled with an asymptotic exponential function and a nonlinear drug effect modeled using a sigmoid Emax relationship to describe the decrease in MDS-UPDRS score (improvement in response) with time. At doses greater than or equal to 10 mg APL-130277, apomorphine concentrations are expected to be near the plateau of the Emax curve for MDS-UPDRS scores, based on a predicted mean Cmax of 3.13 ng/mL associated with a 10 mg dose which is 3-fold higher than the EC50 estimate. However, as illustrated in FIG. 16, there is substantial inter-patient variability in both plasma levels of apomorphine and changes in MDS-UPDRS observed. This suggests a plasma concentration to achieve a full ON will vary by PD subject. Consistent with these findings, the titration data from the clinical studies demonstrated the need for individual dose titration.

Different patients experience full “ON” response at different plasma concentration. The origin of the variability is not known. However, once a plasma level for a full “ON” response in a given patient is achieved, increasing the plasma concentration lengthens the duration of the “ON” response. The effect in patients that achieve full “ON” at a sufficiently low plasma concentration approaches levodopa response. This suggests that in a patient population with sufficient response to low doses of sublingual apomorphine, apomorphine therapy can be used in place of levodopa therapy.

Simulations were conducted using the final E-R model to estimate the apomorphine concentrations required to achieve a full “ON” response. Two different approaches were used for this analysis: 1) the updated population PK model was used to estimate apomorphine concentrations at the time of each observed ON response in the updated dataset and 2) using updated data, a two-stage method that initially estimated a threshold MDS-UPDRS response associated with the change in patient status from OFF to ON followed by estimation of the apomorphine concentration at the time of observed change in MDS-UPDRS score that was equal to or greater than the threshold response. Using a receiver operating characteristic (ROC) analysis, an updated cutoff value of at least 9.5 points relative to the most recent baseline MDS-UPDRS score was associated with a full ON state. Results of both simulations were in good agreement, as were the predicted concentrations between previous and updated analyses. The updated mean (±SD) apomorphine concentration associated with full ON response was 3.39 (±1.90) ng/mL for simulation 1; and 3.18 (±1.91) ng/mL was associated with the observed MDS-UPDRS score change from recent baseline of ≤9.5 points for simulation 2. Similar to the previous analysis, these values are also fairly consistent with the predicted mean Cmax of 3.13 ng/mL for a 10 mg dose of APL-130277 using the updated population PK model.

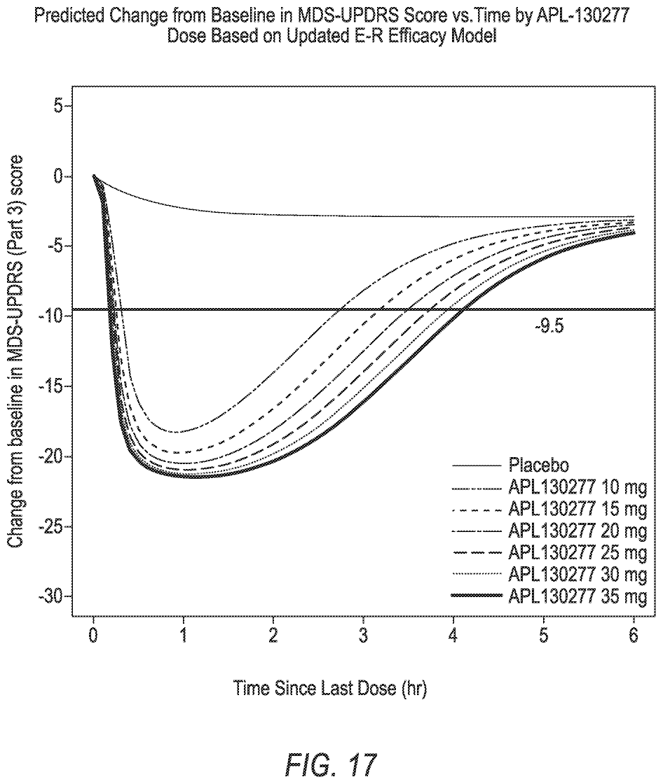

FIG. 17 is a dose-response curve for APL-130277 constructed by simulating the longitudinal change from recent baseline in MDS-UPDRS score across the proposed therapeutic dose range from 10 to 35 mg. This curve was used to update the estimates of onset and duration of response using the threshold 9.5-point or greater drop from most recent baseline MDS-UPDRS score as a predictor of efficacy (ON state). Onset of response was rapid (12 to 18 minutes) at all doses. Duration of response ranged from 2.4 hr for a 10 mg dose to 3.9 hr for a 35 mg dose of APL-130277. As exposure increased, time of onset decreased, while duration and magnitude of response increased.

### CONCLUSIONS

A longitudinal E-R model was updated to describe the decrease in MDS-UPDRS scores (improvement in response) following administration of APL-130277 to PD patients based on data from Phase 2 and 3 studies.

The following conclusions were obtained from the updated analysis:

The E-R model consisted of placebo and drug effects: the placebo effect was described by an asymptotic exponential function and a drug effect component described by an inhibitory sigmoid Emax model with an EC50 of 1.05 ng/mL and sigmoidicity factor (gamma) of 1.7. The model-predicted maximum decrease in MDS-UPDRS score (Emax) was 20 points.

A cutoff of ≤9.5 points in the MDS-UPDRS score was associated with a full ON state. Simulations showed that mean (±SD) apomorphine concentrations of 3.18 (±1.91) ng/mL to 3.39 (±1.90) ng/mL correspond with these conditions, and concentrations are consistent with the predicted apomorphine mean Cmax of 3.13 ng/mL for a 10 mg dose of APL-130277.

Model-predicted onset of response was rapid (12 to 18 minutes) across the dose range of 10 to 35 mg and duration of response increased with increasing dose, from 2.4 hr at 10 mg to 3.9 hr at 35 mg APL-130277.

### ADDITIONAL EMBODIMENTS

Also disclosed herein are embodiments A1-A71.

A1. A method of treating a patient having Parkinson's disease without causing a clinically significant adverse event associated with subcutaneous administration of apomorphine, the method comprising sublingually administering a pharmaceutical composition comprising from about 10 mg to about 60 mg of apomorphine, or a pharmaceutically acceptable salt thereof, to the patient.
A2. A method of treating Parkinson's disease, in a patient in need thereof, without causing a clinically significant adverse event associated with subcutaneous administration of apomorphine, the method comprising sublingually administering a pharmaceutical composition comprising from about 10 mg to about 60 mg of apomorphine, or a pharmaceutically acceptable salt thereof, to the patient.
A3. A method of administering at least 10 mg of apomorphine to a patient having Parkinson's disease causing a clinically significant adverse event associated with subcutaneous administration of apomorphine, the method comprising sublingually administering a pharmaceutical composition comprising at least 10 mg of apomorphine, or a pharmaceutically acceptable salt thereof, to the patient.
A4. A method of minimizing the risk of one or more adverse events associated with subcutaneous administration of apomorphine in a patient having Parkinson's disease comprising sublingually administering a pharmaceutical composition comprising apomorphine, or a pharmaceutically acceptable salt thereof, to the patient.
A5. A method of minimizing one or more adverse events associated with subcutaneous administration of apomorphine in a patient having Parkinson's disease comprising sublingually administering a pharmaceutical composition comprising apomorphine, or a pharmaceutically acceptable salt thereof, to the patient.
A6. A method of treating a patient having Parkinson's disease comprising:

(a) sublingually administering a pharmaceutical composition comprising at least 10 mg of apomorphine, or a pharmaceutically acceptable salt thereof;

(b) monitoring, or having monitored, the patient's QT interval; and

(c) discontinuing use of apomorphine, or a pharmaceutically acceptable salt thereof, if the QT interval is prolonged more than 10 ms.

A7. A method of treating a patient having Parkinson's disease comprising:

(a) selecting a sublingually administered pharmaceutical composition that does not clinically significantly increase QT interval prolongation, wherein the pharmaceutical composition comprises at least 10 mg of apomorphine, or a pharmaceutically acceptable salt thereof;

(b) monitoring, or having monitored, the patient's QT interval; and

(c) discontinuing use of the pharmaceutical composition if the patient demonstrates clinically significant QT interval prolongation.

A8. A method of treating Parkinson's disease with a pharmaceutical composition that provides incidence of QT interval prolongation no greater than that in a placebo-treated group, comprising sublingually administering the pharmaceutical composition to a patient in need thereof, wherein the pharmaceutical composition comprises at least 10 mg of apomorphine, or a pharmaceutically acceptable salt thereof.
A9. A method of treating Parkinson's disease with a pharmaceutical composition without inducing QT interval prolongation greater than that in a placebo-treated group, comprising sublingually administering the pharmaceutical composition to a patient in need thereof, wherein the pharmaceutical composition comprises at least 10 mg of apomorphine, or a pharmaceutically acceptable salt thereof.
A10. A method of treating a patient having an “OFF” episode associated with Parkinson's disease by providing a sustained “ON” response for at least 60 minutes post-administration, the method comprising sublingually administering a pharmaceutical composition comprising from about 10 mg to about 60 mg of apomorphine, or a pharmaceutically acceptable salt thereof, to the patient.
A11. The method of any preceding embodiment, wherein the pharmaceutical composition comprises from about 10 mg to about 60 mg of apomorphine, or a pharmaceutically acceptable salt thereof.
A12. The method of embodiment A11, wherein the pharmaceutical composition comprises about 10 mg of apomorphine, or a pharmaceutically acceptable salt thereof.
A13. The method of embodiment A11, wherein the pharmaceutical composition comprises about 15 mg of apomorphine, or a pharmaceutically acceptable salt thereof.
A14. The method of embodiment A11, wherein the pharmaceutical composition comprises about 20 mg of apomorphine, or a pharmaceutically acceptable salt thereof.
A15. The method of embodiment A11, wherein the pharmaceutical composition comprises about 25 mg of apomorphine, or a pharmaceutically acceptable salt thereof.
A16. The method of embodiment A11, wherein the pharmaceutical composition comprises about 30 mg of apomorphine, or a pharmaceutically acceptable salt thereof.
A17. The method of embodiment A11, wherein the pharmaceutical composition comprises about 35 mg of apomorphine, or a pharmaceutically acceptable salt thereof.
A18. The method of any preceding embodiment, wherein the patient having Parkinson's disease is experiencing motor fluctuations prior to administration.
A19. The method of any preceding embodiment, wherein the patient having Parkinson's disease is experiencing motor fluctuations immediately prior to administration.
A20. The method of any preceding embodiment, wherein the patient having Parkinson's disease is experiencing an “OFF” episode prior to administration.
A21. The method of any preceding embodiment, wherein the patient having Parkinson's disease is experiencing an “OFF” episode immediately prior to administration.
A22. The method of any preceding embodiment, wherein the patient having Parkinson's disease is experiencing end-of-dose wearing “OFF”, early morning “OFF,” partial “OFF,” delayed “OFF,” No-ON “OFF” or unpredictable “OFF” prior to administration.
A23. The method of any preceding embodiment, wherein the patient having Parkinson's disease is experiencing end-of-dose wearing “OFF”, early morning “OFF,” partial “OFF,” delayed “OFF,” No-ON “OFF” or unpredictable “OFF” immediately prior to administration.
A24. The method of any preceding embodiment, wherein the pharmaceutical composition is a sublingual film.
A25. The method of embodiment A24, wherein the sublingual film is a bilayer film having a first layer comprising apomorphine or a pharmaceutically acceptable salt thereof and a second layer comprising a pH-modifier.
A26. The method of embodiment A25, wherein the pH-modifier is pyridoxine hydrochloride.
A27. The method of embodiment A25, wherein the sublingual film further comprises a permeation enhancer.
A28. The method of embodiment A25, wherein the bilayer film comprises a first layer comprising from about 10 mg to about 60 mg of apomorphine or a pharmaceutically acceptable salt thereof and a second layer comprising from about 20 to about 70 wt % pyridoxine.
A29. The method embodiment A24, wherein following sublingual administration, the film produces an average circulating apomorphine plasma concentration of at least 3 ng/mL within 20 minutes.
A30. The method of any preceding embodiment, wherein apomorphine or a pharmaceutically acceptable salt thereof is apomorphine hydrochloride.
A31. The method of embodiment A30, wherein apomorphine or a pharmaceutically acceptable salt thereof is apomorphine hydrochloride hemihydrate.
A32. The method of any one of embodiments A1-A5, wherein the adverse event associated with subcutaneous administration of apomorphine is prolongation of the QT interval.
A33. The method of any one of embodiments A6-A9 and A32, wherein the mean change from baseline in QTcF in the patient is no greater than 10 ms.
A34. The method of any one of embodiments A6-9 and 32, wherein the mean change from baseline in QTcF in the patient is no greater than 5 ms.
A35. The method of any one of embodiments A6-9 and 32, wherein the mean change from baseline in QTcF in the patient is no greater than about 3.3 ms.
A36. The method of any one of embodiments A6-9 and 32, wherein the mean change from baseline in QTcF in the patient 60 minutes after administration is not greater than about 3.3 ms.
A37. The method of any one of embodiments A6-9 and 32, wherein the time-matched and placebo-adjusted mean change from baseline in QTcF in the patient is not greater than 10 ms.
A38. The method of any one of embodiments A6-9 and 32, wherein the time-matched and placebo-adjusted mean change from baseline in QTcF in the patient is not greater than about 6.2 ms.
A39. The method of any one of embodiments A6-A9 and A32, wherein the time-matched and placebo-adjusted mean change from baseline in QTcF in the patient 60 minutes after administration is not greater than about 6.2 ms.
A40. The method of any one of embodiments A6-A9 and A32, wherein the patient does not experience a QTcF greater than 500 ms.
A41. The method of any one of embodiments A6-A9 and A32, wherein the patient does not experience a QTcF greater than 480 ms.
A42. The method of any one of embodiments A6-A9 and A32, wherein the patient does not have a clinically significant risk of experiencing a QTcF greater than 450 ms.
A43. The method of any one of embodiments A6-A9 and A32, wherein the patient does not experience an increase in QTcF greater than 60 ms.
A44. The method of any one of embodiments A6-A9 and A32, wherein the patient does not have a clinically significant risk of experiencing an increase in QTcF of greater than 30 ms.
A45. The method of any one of embodiments A1-A5, wherein the adverse event associated with subcutaneous administration of apomorphine is selected from prolongation of the QT interval, orthostatic hypotension, syncope, dyskinesia, hallucinations, and impulse control problems, or any combination thereof.
A46. The method of embodiment A45, wherein the adverse event associated with subcutaneous administration of apomorphine is prolongation of the QT interval.
A47. The method of embodiment A46, wherein the patient's risk of prolongation of the QT interval from the treatment is no greater than 2%.
A48. The method of embodiment A45, wherein the adverse event associated with subcutaneous administration of apomorphine is orthostatic hypotension.
A49. The method of embodiment A48, wherein the patient's risk of orthostatic hypotension from the treatment is no greater than 2%.
A50. The method of embodiment A45, wherein the adverse event associated with subcutaneous administration of apomorphine is syncope.
A51. The method of embodiment A50, wherein the patient's risk of syncope from the treatment is no greater than 2%.
A52. The method of embodiment 45, wherein the adverse event associated with subcutaneous administration of apomorphine is dyskinesia.
A53. The method of embodiment A52, wherein the patient's risk of dyskinesia from the treatment is no greater than 2%.
A54. The method of embodiment A45, wherein the adverse event associated with subcutaneous administration of apomorphine is hallucinations.
A55. The method of embodiment A54, wherein the patient's risk of hallucinations from the treatment is no greater than 2%.
A56. The method of embodiment A45, wherein the adverse event associated with subcutaneous administration of apomorphine is impulse control problems.
A57. The method of embodiment A56, wherein the patient's risk of impulse control problems from the treatment is no greater than 2%.
A58. The method of any preceding embodiment, wherein the methods results in a reduction from baseline in MDS-UPDRS Part III motor examination score of at least 5 when measured 30 minutes post-dose.
A59. The method of any preceding embodiment, wherein the methods results in a reduction from baseline in MDS-UPDRS Part III motor examination score of at least 5 when measured 90 minutes post-dose.
A60. The method of any preceding embodiment, wherein the methods results in a reduction from baseline in MDS-UPDRS Part III motor examination score of at least 10 when measured 30 minutes post-dose.
A61. The method of any preceding embodiment, wherein the methods results in a reduction from baseline in MDS-UPDRS Part III motor examination score of at least about 11.1 when measured 30 minutes post-dose.
A62. The method of any preceding embodiment, wherein the method provides a sustained “ON” response in the patient for at least 60 minutes post-administration.
A63. The method of embodiment A62, wherein the method provides a sustained “ON” response in the patient for at least 75 minutes post-administration.
A64. The method of embodiment A63, wherein the method provides a sustained “ON” response in the patient for at least 90 minutes post-administration.
A65. A method of treating a patient having an “OFF” episode associated with Parkinson's disease, the method comprising sublingually administering to the patient a first pharmaceutical composition comprising a first dose of apomorphine or a pharmaceutically acceptable salt thereof; and sublingually administering a second pharmaceutical composition comprising a second dose of apomorphine or a pharmaceutically acceptable salt thereof; wherein the first dose and the second dose are consecutive, and at least 2 hours separate administration of the first dose and the second dose.
A66. The method of embodiment A65, wherein the second dose is greater than the first dose if the patient does not respond to the first dose.
A67. The method of embodiment A65 or A66, wherein the first dose comprises about 10 mg of apomorphine or a pharmaceutically acceptable salt thereof.
A68. The method of embodiment A67, wherein if the first dose is ineffective, a second dose is not given for the “OFF” episode.
A69. The method of any one of embodiments A64-A67, wherein the first pharmaceutical composition is a sublingual film comprising a first portion comprising apomorphine particles comprising an acid addition salt of apomorphine, and a second portion comprising a pH neutralizing agent; and the second pharmaceutical composition is a sublingual film comprising a first portion comprising apomorphine particles comprising an acid addition salt of apomorphine, and a second portion comprising a pH neutralizing agent.
A70. A method of treating a patient having an “OFF” episode associated with Parkinson's disease, the method comprising:
(a) sublingually administering to the patient having a first “OFF” episode a first dose of a sublingual film comprising apomorphine particles comprising an acid addition salt of apomorphine and a second portion comprising a pH neutralizing agent;
(b) if the patient does not respond to the first dose, sublingually administering to the patient having a subsequent “OFF” episode a subsequent dose of the sublingual film, wherein the subsequent dose comprises more of the acid addition salt of apomorphine than the first dose;
(c) if the patient in step (b) does not respond, repeating step (b) such that each subsequent dose comprises more of the acid addition salt of apomorphine than the preceding dose until the patient responds to the treatment; and
(d) sublingually administering to the patient a therapeutically effective dose of the sublingual film to the patient;
wherein the dose identified in steps (a), (b), or (c) to which the patient responds is the therapeutically effective dose;
wherein no more than one dose is administered per “OFF” episode in each of steps (a), (b), and (c); and
wherein at least 2 hours separate administration of two consecutive doses of the sublingual films to the patient.
A71. The method of any preceding embodiment, wherein no 5HT3 antagonist is administered concomitantly.

Also disclosed herein are embodiments B1-B23.

B1. A method of improving motor function in a patient having an “OFF” episode associated with Parkinson's disease, the method comprising administering to the patient a supratherapeutic dose of apomorphine or a pharmaceutically acceptable salt thereof.
B2. A method of treating a patient having an “OFF” episode associated with Parkinson's disease, the method comprising administering to the patient a supratherapeutic dose of apomorphine or a pharmaceutically acceptable salt thereof.
B3. The method of embodiment B1 or B2, wherein the administering step improves motor function of the patient, as measured by an improvement in a Movement Disorder Society Unified Parkinson's Disease Rating Scale Part III Motor Examination (MDS-UPDRS Part III) score in the patient after a period of 30 to 90 minutes following the administering step relative to the MDS-UPDRS III score measured prior to the administering step.
B4. The method of embodiment B3, wherein the improvement is at least a 10% reduction in the MDS-UPDRS Part III score relative to the MDS-UPDRS III score measured prior to the administering step.
B5. The method of any one of embodiments B1 to B4, wherein the administering step improves motor function of the patient, as measured by an improvement in a Movement Disorder Society Unified Parkinson's Disease Rating Scale Part III Motor Examination (MDS-UPDRS Part III) score in the patient after a period of 30 to 90 minutes following the administering step, wherein the improvement is greater than an improvement in the MDS-UPDRS Part III score after the same period following administration of a therapeutic dose of apomorphine or a pharmaceutically acceptable salt thereof to the patient.
B6. The method of embodiment B5, wherein the MDS-UPDRS Part III in the patient after a period of 30 to 90 minutes following the administering step is at least 10% lower than the MDS-UPDRS Part III after the same period following administration of the therapeutic dose of apomorphine or a pharmaceutically acceptable salt thereof to the patient.
B7. The method of any one of embodiments B1 to B6, wherein the supratherapeutic dose comprises 5 to 20 mg more of apomorphine or a pharmaceutically acceptable salt thereof than a therapeutic dose.
B8. The method of embodiment B7, wherein the supratherapeutic dose comprises 5 mg more of apomorphine or a pharmaceutically acceptable salt thereof than the therapeutic dose.
B9. The method of embodiment B7, wherein the supratherapeutic dose comprises 10 mg more of apomorphine or a pharmaceutically acceptable salt thereof than the therapeutic dose.
B10. The method of embodiment B7, wherein the supratherapeutic dose comprises 15 mg more of apomorphine or a pharmaceutically acceptable salt thereof than the therapeutic dose.
B11. The method of embodiment B7, wherein the supratherapeutic dose comprises 20 mg more of apomorphine or a pharmaceutically acceptable salt thereof than the therapeutic dose.
B12. The method of any one of embodiments B1 to B11, wherein the supratherapeutic dose comprises 15 mg to 60 mg of apomorphine or a pharmaceutically acceptable salt thereof.
B13. The method of embodiment B12, wherein the supratherapeutic dose is 15 mg, 20 mg, 25 mg, 30 mg, 35 mg, 40 mg, 50 mg, or 60 mg of apomorphine or a pharmaceutically acceptable salt thereof.
B14. The method of any one of embodiments B1 to B13, wherein the administering step produces a FULL “ON” state in the patient.
B15. The method of any one of embodiments B1 to B14, wherein apomorphine or a pharmaceutically acceptable salt thereof is sublingually administering to the patient.
B16. The method of any one of embodiments B1 to B15, wherein apomorphine or a pharmaceutically acceptable salt thereof is administered to the patient in a unit dosage form.
B17. The method of embodiment B16, wherein the unit dosage form is a lozenge, a pill, a tablet, a film, or a capsule.
B18. The method of embodiment B17, wherein the unit dosage form is a film.
B19. The method of any one of embodiments B16 to B18, wherein the unit dosage form comprises a first portion comprising a pharmaceutically acceptable salt of apomorphine and a second portion comprising a pH neutralizing agent.
B20. The method of embodiment B19, wherein the pH neutralizing agent is pyridoxine.
B21. The method of embodiment B19 or B20, wherein the first portion comprises apomorphine particles comprising a pharmaceutically acceptable salt of apomorphine.
B22. The method of any one of embodiments B1 to B21, wherein the pharmaceutically acceptable salt of apomorphine is an acid addition salt of apomorphine.
B23. The method of embodiment B22, wherein the acid addition salt of apomorphine is apomorphine hydrochloride.

Also disclosed herein are embodiments C1-15.

C1. A method of treating Parkinson's disease in a patient receiving a levodopa regimen, the method comprising administering to the patient a therapeutically effective amount of apomorphine or a pharmaceutically acceptable salt thereof, wherein the administering step supplants the first levodopa dose of the day.
C2. The method of embodiment C1 further comprising administering the second and further levodopa doses of the day after administering the therapeutically effective amount of apomorphine or a pharmaceutically acceptable salt thereof.
C3. A method of treating Parkinson's disease in a patient, the method comprising administering to the patient a therapeutically effective amount of apomorphine or a pharmaceutically acceptable salt thereof as a monotherapy or a combination therapy, the combination therapy excluding levodopa.
C4. The method of any one of embodiments C1 to C3 further comprising administering carbidopa and/or entacapone.
C5. The method of any one of embodiments C1 to C4, wherein the therapeutically effective amount is 10 mg to 60 mg of apomorphine or a pharmaceutically acceptable salt thereof.
C6. The method of embodiment C5, wherein the therapeutically effective amount is 10 mg, 15 mg, 20 mg, 25 mg, 30 mg, 35 mg, 40 mg, 50 mg, or 60 mg of apomorphine or a pharmaceutically acceptable salt thereof.
C7. The method of any one of embodiments C1 to C6, wherein apomorphine or a pharmaceutically acceptable salt thereof is administered sublingually to the patient.
C8. The method of any one of embodiments C1 to C7, wherein apomorphine or a pharmaceutically acceptable salt thereof is administered to the patient in a unit dosage form.
C9. The method of embodiment C8, wherein the unit dosage form is a lozenge, a pill, a tablet, a film, or a capsule.
C10. The method of embodiment C9, wherein the unit dosage form is a film.
C11. The method of any one of embodiments C8 to C10, wherein the unit dosage form comprises a first portion comprising a pharmaceutically acceptable salt of apomorphine and a second portion comprising a pH neutralizing agent.
C12. The method of embodiment C11, wherein the pH neutralizing agent is pyridoxine.
C13. The method of embodiment C11 or C12, wherein the first portion comprises apomorphine particles comprising a pharmaceutically acceptable salt of apomorphine.
C14. The method of any one of embodiments C1 to C13, wherein the pharmaceutically acceptable salt of apomorphine is an acid addition salt of apomorphine.
C15. The method of embodiment C14, wherein the acid addition salt of apomorphine is apomorphine hydrochloride.

Also disclosed herein are embodiments D1-D70.

D1. A method of treating a patient having Parkinson's disease without causing a clinically significant adverse event associated with subcutaneous administration of apomorphine, the method comprising sublingually administering a pharmaceutical composition comprising from about 10 mg to about 60 mg of apomorphine, or a pharmaceutically acceptable salt thereof, to the patient.
D2. A method of treating Parkinson's disease, in a patient in need thereof, without causing a clinically significant adverse event associated with subcutaneous administration of apomorphine, the method comprising sublingually administering a pharmaceutical composition comprising from about 10 mg to about 60 mg of apomorphine, or a pharmaceutically acceptable salt thereof, to the patient.
D3. A method of administering at least 10 mg of apomorphine to a patient having Parkinson's disease without causing a clinically significant adverse event associated with subcutaneous administration of apomorphine, the method comprising sublingually administering a pharmaceutical composition comprising at least 10 mg of apomorphine, or a pharmaceutically acceptable salt thereof, to the patient.
D4. A method of minimizing the risk of one or more adverse events associated with subcutaneous administration of apomorphine in a patient having Parkinson's disease comprising sublingually administering a pharmaceutical composition comprising apomorphine, or a pharmaceutically acceptable salt thereof, to the patient.
D5. A method of minimizing one or more adverse events associated with subcutaneous administration of apomorphine in a patient having Parkinson's disease comprising sublingually administering a pharmaceutical composition comprising apomorphine, or a pharmaceutically acceptable salt thereof, to the patient.
D6. A method of treating a patient having Parkinson's disease comprising:

(a) sublingually administering a pharmaceutical composition comprising at least 10 mg of apomorphine, or a pharmaceutically acceptable salt thereof;

(b) monitoring, or having monitored, the patient's QT interval; and

(c) discontinuing use of apomorphine, or a pharmaceutically acceptable salt thereof, if the QT interval is prolonged more than 10 ms.

D7. A method of treating a patient having Parkinson's disease comprising:

(a) selecting a sublingually administered pharmaceutical composition that does not clinically significantly increase QT interval prolongation, wherein the pharmaceutical composition comprises at least 10 mg of apomorphine, or a pharmaceutically acceptable salt thereof;

(b) monitoring, or having monitored, the patient's QT interval; and

(c) discontinuing use of the pharmaceutical composition if the patient demonstrates clinically significant QT interval prolongation.

D8. A method of treating Parkinson's disease with a pharmaceutical composition that provides incidence of QT interval prolongation no greater than that in a placebo-treated group, comprising sublingually administering the pharmaceutical composition to a patient in need thereof, wherein the pharmaceutical composition comprises at least 10 mg of apomorphine, or a pharmaceutically acceptable salt thereof.
D9. A method of treating Parkinson's disease with a pharmaceutical composition without inducing QT interval prolongation greater than that in a placebo-treated group, comprising sublingually administering the pharmaceutical composition to a patient in need thereof, wherein the pharmaceutical composition comprises at least 10 mg of apomorphine, or a pharmaceutically acceptable salt thereof.
D10. A method of treating a patient having an “OFF” episode associated with Parkinson's disease by providing a sustained “ON” response for at least 60 minutes post-administration, the method comprising sublingually administering a pharmaceutical composition comprising from about 10 mg to about 60 mg of apomorphine, or a pharmaceutically acceptable salt thereof, to the patient.
D11. The method of any preceding embodiment D1-D10, wherein the pharmaceutical composition comprises from about 10 mg to about 60 mg of apomorphine, or a pharmaceutically acceptable salt thereof.
D12. The method of embodiment D11, wherein the pharmaceutical composition comprises about 10 mg of apomorphine, or a pharmaceutically acceptable salt thereof.
D13. The method of embodiment D11, wherein the pharmaceutical composition comprises about 15 mg of apomorphine, or a pharmaceutically acceptable salt thereof.
D14. The method of embodiment D11, wherein the pharmaceutical composition comprises about 20 mg of apomorphine, or a pharmaceutically acceptable salt thereof.
D15. The method of embodiment D11, wherein the pharmaceutical composition comprises about 25 mg of apomorphine, or a pharmaceutically acceptable salt thereof.
D16. The method of embodiment D11, wherein the pharmaceutical composition comprises about 30 mg of apomorphine, or a pharmaceutically acceptable salt thereof.
D17. The method of embodiment D11, wherein the pharmaceutical composition comprises about 35 mg of apomorphine, or a pharmaceutically acceptable salt thereof.
D18. The method of any preceding embodiment D1-D17, wherein the patient having Parkinson's disease is experiencing motor fluctuations prior to administration.
D19. The method of any preceding embodiment D1-D18, wherein the patient having Parkinson's disease is experiencing motor fluctuations immediately prior to administration.
D20. The method of any preceding embodiment D1-D19, wherein the patient having Parkinson's disease is experiencing an “OFF” episode prior to administration.
D21. The method of any preceding embodiment D1-D20, wherein the patient having Parkinson's disease is experiencing an “OFF” episode immediately prior to administration.
D22. The method of any preceding embodiment D1-D21, wherein the patient having Parkinson's disease is experiencing end-of-dose wearing “OFF”, early morning “OFF,” partial “OFF,” delayed “OFF,” No-ON “OFF” or unpredictable “OFF” prior to administration.
D23. The method of any preceding embodiment D1-D22, wherein the patient having Parkinson's disease is experiencing end-of-dose wearing “OFF”, early morning “OFF,” partial “OFF,” delayed “OFF,” No-ON “OFF” or unpredictable “OFF” immediately prior to administration.
D24. The method of any preceding embodiment D1-D23, wherein the pharmaceutical composition is a sublingual film.
D25. The method of embodiment D24, wherein the sublingual film is a bilayer film having a first layer comprising apomorphine or a pharmaceutically acceptable salt thereof and a second layer comprising a pH-modifier.
D26. The method of embodiment D25, wherein the pH-modifier is pyridoxine hydrochloride.
D27. The method of embodiment D25, wherein the sublingual film further comprises a permeation enhancer.
D28. The method of embodiment D25, wherein the bilayer film comprises a first layer comprising from about 10 mg to about 60 mg of apomorphine or a pharmaceutically acceptable salt thereof and a second layer comprising from about 20 to about 70 wt % pyridoxine.
D29. The method embodiment D24, wherein following sublingual administration, the film produces an average circulating apomorphine plasma concentration of at least 3 ng/mL within 20 minutes.
D30. The method of any preceding embodiment D1-D29, wherein apomorphine or a pharmaceutically acceptable salt thereof is apomorphine hydrochloride.
D31. The method of embodiment D30, wherein apomorphine or a pharmaceutically acceptable salt thereof is apomorphine hydrochloride hemihydrate.
D32. The method of any one of embodiments D1-D5, wherein the adverse event associated with subcutaneous administration of apomorphine is prolongation of the QT interval.
D33. The method of any one of embodiments D6-D9 and D32, wherein the mean change from baseline in QTcF in the patient is no greater than 10 ms.
D34. The method of any one of embodiments D6-D9 and D32, wherein the mean change from baseline in QTcF in the patient is no greater than 5 ms.
D35. The method of any one of embodiments D6-D9 and D32, wherein the mean change from baseline in QTcF in the patient is no greater than about 3.3 ms.
D36. The method of any one of embodiments D6-D9 and D32, wherein the mean change from baseline in QTcF in the patient 60 minutes after administration is not greater than about 3.3 ms.
D37. The method of any one of embodiments D6-D9 and D32, wherein the time-matched and placebo-adjusted mean change from baseline in QTcF in the patient is not greater than 10 ms.
D38. The method of any one of embodiments D6-D9 and D32, wherein the time-matched and placebo-adjusted mean change from baseline in QTcF in the patient is not greater than about 6.2 ms.
D39. The method of any one of embodiments D6-D9 and D32, wherein the time-matched and placebo-adjusted mean change from baseline in QTcF in the patient 60 minutes after administration is not greater than about 6.2 ms.
D40. The method of any one of embodiments D6-D9 and D32, wherein the patient does not experience a QTcF greater than 500 ms.
D41. The method of any one of embodiments D6-D9 and D32, wherein the patient does not experience a QTcF greater than 480 ms.
D42. The method of any one of embodiments D6-D9 and D32, wherein the patient does not have a clinically significant risk of experiencing a QTcF greater than 450 ms.
D43. The method of any one of embodiments D6-D9 and D32, wherein the patient does not experience an increase in QTcF greater than 60 ms.
D44. The method of any one of embodiments D6-D9 and D32, wherein the patient does not have a clinically significant risk of experiencing an increase in QTcF of greater than 30 ms.
D45. The method of any one of embodiments D1-D5, wherein the adverse event associated with subcutaneous administration of apomorphine is selected from prolongation of the QT interval, orthostatic hypotension, syncope, dyskinesia, hallucinations, and impulse control problems, or any combination thereof.
D46. The method of embodiment D45, wherein the adverse event associated with subcutaneous administration of apomorphine is prolongation of the QT interval.
D47. The method of embodiment D46, wherein the patient's risk of prolongation of the QT interval from the treatment is no greater than 2%.
D48. The method of embodiment D45, wherein the adverse event associated with subcutaneous administration of apomorphine is orthostatic hypotension.
D49. The method of embodiment D48, wherein the patient's risk of orthostatic hypotension from the treatment is no greater than 2%.
D50. The method of embodiment D45, wherein the adverse event associated with subcutaneous administration of apomorphine is syncope.
D51. The method of embodiment D50, wherein the patient's risk of syncope from the treatment is no greater than 2%.
D52. The method of embodiment D45, wherein the adverse event associated with subcutaneous administration of apomorphine is dyskinesia.
D53. The method of embodiment D52, wherein the patient's risk of dyskinesia from the treatment is no greater than 2%.
D54. The method of embodiment D45, wherein the adverse event associated with subcutaneous administration of apomorphine is hallucinations.
D55. The method of embodiment D54, wherein the patient's risk of hallucinations from the treatment is no greater than 2%.
D56. The method of embodiment D45, wherein the adverse event associated with subcutaneous administration of apomorphine is impulse control problems.
D57. The method of embodiment D56, wherein the patient's risk of impulse control problems from the treatment is no greater than 2%.
D58. The method of any preceding embodiment D1-D57, wherein the methods results in a reduction from baseline in MDS-UPDRS Part III motor examination score of at least 5 when measured 30 minutes post-dose.
D59. The method of any preceding embodiment D1-D58, wherein the methods results in a reduction from baseline in MDS-UPDRS Part III motor examination score of at least 10 when measured 30 minutes post-dose.
D60. The method of any preceding embodiment D1-D59, wherein the methods results in a reduction from baseline in MDS-UPDRS Part III motor examination score of at least about 11.1 when measured 30 minutes post-dose.
D61. The method of any preceding embodiment D1-D60, wherein the method provides a sustained “ON” response in the patient for at least 60 minutes post-administration.
D62. The method of embodiment D61, wherein the method provides a sustained “ON” response in the patient for at least 75 minutes post-administration.
D63. The method of embodiment D62, wherein the method provides a sustained “ON” response in the patient for at least 90 minutes post-administration.
D64. A method of treating a patient having an “OFF” episode associated with Parkinson's disease, the method comprising sublingually administering to the patient a first pharmaceutical composition comprising a first dose of apomorphine or a pharmaceutically acceptable salt thereof; and sublingually administering a second pharmaceutical composition comprising a second dose of apomorphine or a pharmaceutically acceptable salt thereof; wherein the first dose and the second dose are consecutive, and at least 2 hours separate administration of the first dose and the second dose.
D65. The method of embodiment D64, wherein the second dose is greater than the first dose if the patient does not respond to the first dose.
D66. The method of embodiment D64 or D65, wherein the first dose comprises about 10 mg of apomorphine or a pharmaceutically acceptable salt thereof.
D67. The method of embodiment D66, wherein if the first dose is ineffective, a second dose is not given for the “OFF” episode.
D68. The method of any one of embodiments D64-D67, wherein the first pharmaceutical composition is a sublingual film comprising a first portion comprising apomorphine particles comprising an acid addition salt of apomorphine, and a second portion comprising a pH neutralizing agent; and the second pharmaceutical composition is a sublingual film comprising a first portion comprising apomorphine particles comprising an acid addition salt of apomorphine, and a second portion comprising a pH neutralizing agent.
D69. A method of treating a patient having an “OFF” episode associated with Parkinson's disease, the method comprising:

(a) sublingually administering to the patient having a first “OFF” episode a first dose of a sublingual film comprising apomorphine particles comprising an acid addition salt of apomorphine and a second portion comprising a pH neutralizing agent;

(b) if the patient does not respond to the first dose, sublingually administering to the patient having a subsequent “OFF” episode a subsequent dose of the sublingual film, wherein the subsequent dose comprises more of the acid addition salt of apomorphine than the first dose;

(c) if the patient in step (b) does not respond, repeating step (b) such that each subsequent dose comprises more of the acid addition salt of apomorphine than the preceding dose until the patient responds to the treatment; and

(d) sublingually administering to the patient a therapeutically effective dose of the sublingual film to the patient;

wherein the dose identified in steps (a), (b), or (c) to which the patient responds is the therapeutically effective dose;

wherein no more than one dose is administered per “OFF” episode in each of steps (a), (b), and (c); and

wherein at least 2 hours separate administration of two consecutive doses of the sublingual films to the patient.

D70. The method of any preceding embodiment D1-D69, wherein no 5HT3 antagonist is administered concomitantly.

### OTHER EMBODIMENTS

Various modifications and variations of the described disclosure will be apparent to those skilled in the art without departing from the scope and spirit of the disclosure. Although the disclosure has been described relating to specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments accordingly are within the claims.

What is claimed is:

1. A method of treating a patient having “OFF” episodes associated with Parkinson's disease, the method comprising sublingually administering to the patient a first pharmaceutical composition comprising a first dose of apomorphine or a pharmaceutically acceptable salt thereof; and sublingually administering a second pharmaceutical composition comprising a second dose of apomorphine or a pharmaceutically acceptable salt thereof; wherein the first dose and the second dose are consecutive, and at least 2 hours separate administration of the first dose and the second dose, wherein the second dose is not given for the same “OFF” episode.

2. The method of claim 1, wherein the second dose is greater than the first dose if the patient does not respond to the first dose.

3. The method of claim 1, wherein the first dose comprises about 10 mg of apomorphine or a pharmaceutically acceptable salt thereof.

4. The method of claim 1, wherein the first pharmaceutical composition is a sublingual film comprising a first portion comprising apomorphine particles comprising an acid addition salt of apomorphine, and a second portion comprising a pH neutralizing agent; and the second pharmaceutical composition is a sublingual film comprising a first portion comprising apomorphine particles comprising an acid addition salt of apomorphine, and a second portion comprising a pH neutralizing agent.

5. The method of claim 1, wherein no 5HT3 antagonist is administered concomitantly.

6. The method of claim 1, wherein the “OFF” episode” is an acute or intermittent “OFF” episode.

7. The method of claim 1, wherein the method has a low incidence of QT prolongation.

8. The method of claim 1, wherein the mean change from baseline in QTcF in the patient is no greater than 10 ms.

9. The method of claim 1, wherein the patient does not experience a QTcF greater than 500 ms.

10. A method of treating a patient having “OFF” episodes associated with Parkinson's disease, the method comprising:
(a) sublingually administering to the patient having a first “OFF” episode a first dose of a sublingual film comprising a first portion comprising apomorphine particles comprising an acid addition salt of apomorphine and a second portion comprising a pH neutralizing agent;
(b) if the patient does not respond to the first dose, sublingually administering to the patient having a subsequent “OFF” episode a subsequent dose of the sublingual film, wherein the subsequent dose comprises more of the acid addition salt of apomorphine than the first dose;
(c) if the patient in step (b) does not respond, repeating step (b) such that each subsequent dose comprises more of the acid addition salt of apomorphine than the preceding dose until the patient responds to the treatment; and
(d) sublingually administering to the patient a therapeutically effective dose of the sublingual film to the patient;
wherein the dose identified in steps (a), (b), or (c) to which the patient responds is the therapeutically effective dose;
wherein no more than one dose is administered per “OFF” episode in each of steps (a), (b), and (c); and
wherein at least 2 hours separate administration of two consecutive doses of the sublingual films to the patient.

11. The method of claim 10, wherein no 5HT3 antagonist is administered concomitantly.

12. The method of claim 10, wherein the “OFF” episode” is an acute or intermittent “OFF” episode.

13. The method of claim 10, wherein the method has a low incidence of QT prolongation.

14. The method of claim 10, wherein the mean change from baseline in QTcF in the patient is no greater than 10 ms.

15. The method of claim 10, wherein the patient does not experience a QTcF greater than 500 ms.

16. The method of claim 10, wherein the first dose comprises about 10 mg of apomorphine or a pharmaceutically acceptable salt thereof.
